(19) United States
(12) Patent Application Publication
(10) Pub. No.: US 2025/0295748 A1
(43) Pub. Date: Sep. 25, 2025

(54) TREATMENT AND PREVENTION OF VIRAL INFECTIONS

(71) Applicant: Sporegen Limited, London (GB)

(72) Inventor: Simon CUTTING, London (GB)

(73) Assignee: Sporegen Limited, London (GB)

(21) Appl. No.: 18/043,631

(22) PCT Filed: Sep. 2, 2021
(86) PCT No.: PCT/GB2021/052268

§ 371 Date: Mar. 1, 2023

(30) Foreign Application Priority Data
Sep. 3, 2020 (GB) 2013874.9

(51) Int. Cl. A61K 39/07; A61K 39/00; A61K 39/215; A61P 31/14

(52) U.S. Cl. CPC A61K 39/07; A61K 39/215; A61P 31/14; A61K 2039/521; A61K 2039/543; A61K 2039/55594

(57) ABSTRACT

The invention relates to bacteria and bacteria-related compositions in formulations for use in treating, preventing or ameliorating viral infections and, in particular, respiratory virus infections acting as antiviral formulations. The invention includes pharmaceutical compositions comprising such formulations, and their use as an immune stimulant or an innate immunity stimulant in immuno-prophylaxis against viral infections. The invention also covers the use of the formulations as an adjuvant, and in vaccinating (i.e. in adaptive and/or acquired immunity) against viral infections.

### CROSS-REFERENCE TO RELATED APPLICATIONS

This application is a 371 National Stage filing and claims the benefit under 35 U.S.C. § 120 to International Application No. PCT/GB2021/052268, filed 2 Sep. 2021, which claims priority to Great Britain Application No. GB2013874.9, filed 3 Sep. 2020, each of which is incorporated herein by reference in its entirety.

### SEQUENCE LISTING

This application herein incorporates by reference in its entirety the Sequence Listing material in the ASCII text file named “97128PCT1 sequence listing”, created Feb. 20, 2023 and having the size of 76 kb filed with this application via EFS-Web.

The present invention relates to the treatment and prevention of viral infections. In particular, the invention relates to bacteria and bacteria-related compositions in formulations for use in treating, preventing or ameliorating viral infections and, in particular, respiratory virus infections (i.e., antiviral formulations). The invention extends to pharmaceutical compositions comprising such formulations, and their use as an immune stimulant or an innate immunity stimulant in immuno-prophylaxis against viral infections. The invention also extends to the use of the formulations as an adjuvant, and in vaccinating (i.e., in adaptive and/or acquired immunity) against viral infections. The formulations are ideally mucosally (e.g., nasally) administerable.

Innate immunity is man's first line of defence against pathogens. This form of immunity is rapidly acquired following exposure and usually occurs in <7 days. Innate immunity is non-specific and will act against a variety of viral and bacterial pathogens and can include multiple factors, for example, inflammation resulting from the activation of macrophages and dendritic cells (DC). This occurs by interaction of the pathogen with pattern recognition receptors on the cell's surface (e.g., Toll-like receptors, TLRs), which leads to the production of cytokines that recruit leukocytes and neutrophils to the infection site. The innate immune response also helps trigger our adaptive immunity, i.e., an antigen-specific response that retains memory should we encounter the pathogen again.

Many viruses have developed complex mechanisms to evade the innate immune response as a prelude to infection. Relevant examples include SARS-CoV1 the causative agents of SARS [1], SARS-Cov2 the causative agent of COVID-19, Respiratory Syncytial Virus (RSV) [13], Rhinovirus [14] and influenza [2]. Boosting innate immunity might, therefore, be one way to enhance resistance to viral pathogens. Indeed, in the case of influenza, agonists of TLRs have been shown to provide protection to disease by interfering with the normal interaction of virus with its host receptor [3].

Agonists are molecules present on bacteria or viruses that normally interact with receptors (typically TLRs) on the surface of host cells (e.g., macrophages and DCs). These molecules can also be found on the surface of non-pathogens. Remarkably, bacterial spores also carry related molecules on their surface. Spores of Bacillus species are found typically in soil and we are exposed to them on a regular basis. Bacillus spores are also in use worldwide as probiotics, i.e., beneficial bacteria that confer health benefits to the host [4].

There is a need in the art for improved formulations for treating or preventing viral infections, and especially infections of respiratory viruses.

The inventor has hypothesized that the application (e.g., nasal or sublingual or by injection, but especially intranasally) of bacterial spores or vegetative cells, or material derived from bacterial spores/cells, may confer protection from, and vaccinate against, infections of viruses in general, and especially of respiratory viruses, such as SARS-Cov-2, whose infectivity correlates with innate immunity.

Thus, in a first aspect of the invention, there is provided a live or dead bacterial spore, a live or dead vegetative bacterium, extracellular material produced by the live cell, or a disrupted bacterial cell homogenate, for use in treating, preventing or ameliorating a virus infection.

In a second aspect of the invention, there is provided a method of treating a virus infection, the method comprising administering, or having administered, to a patient in need of such treatment, a therapeutically effective amount of a live or dead bacterial spore, a live or dead vegetative bacterium, extracellular material produced by the live cell, or a disrupted bacterial cell homogenate.

In another aspect of the invention, there is provided a live or dead bacterial spore, a live or dead vegetative bacterium, extracellular material produced by the live cell, or a disrupted bacterial cell homogenate, for use as an innate immunity stimulant in immuno-prophylaxis against a viral infection.

In yet another aspect, there is provided a method of stimulating innate immunity in immuno-prophylaxis against a viral infection, the method comprising administering, or having administered, to a patient in need of such treatment, a therapeutically effective amount of a live or dead bacterial spore, a live or dead vegetative bacterium, extracellular material produced by the live cell, or a disrupted bacterial cell homogenate.

Advantageously, as shown in Example 1, the inventors have surprisingly shown that Bacillus spores elicit innate immunity in mice, sufficient to protect against respiratory virus infections. Accordingly, and preferably, the live or dead bacterial spore, live or dead vegetative bacterium or the extracellular material/homogenate therefrom can be used as a suitable prophylactic against viral infections by eliciting an innate immune response. Innate immune responses tend to be non-specific and so are good for combatting mutant forms of viruses (for example, viral strain variants). Accordingly, the live or dead bacterial spore, a live or dead vegetative bacterium, extracellular material produced by the live cell, or a disrupted bacterial cell homogenate may be viewed as being an immune stimulant, an innate immunity stimulant or a prophylactic immune modulator.

Furthermore, bacterial spores are particularly advantageous because they can be produced simply using growth in bioreactors. Furthermore, spores can be stored in liquid or desiccated form at most temperatures<600° C. and have an almost indefinite shelf-life, enabling stockpiling (of particular value in a pandemic situation).

In one embodiment, it is preferred that a live bacterial spore is used to combat the viral infection. As shown in Example 5, boosting mice with purified live spores of Bacillus subtilis augments immunity by increasing the titre of antigen-specific SIgA in the lungs and saliva, as well as IgG in serum. The ability to augment or stimulate mucosal responses, such as SIgA, is important for existing coronavirus vaccines and demonstrates that spores surprisingly exert a unique and novel adjuvant effect on an antigen administered by a parenteral route. As such, this data demonstrates that spores have utility for improving existing coronavirus vaccines by enhancing their performance and potentiating the immune response.

Accordingly, preferably the the live or dead bacterial spore, the live or dead vegetative bacterium, extracellular material produced by the live cell, or the disrupted bacterial cell homogenate is adapted to exert an adjuvant effect on an antigen administered parenterally. The parenterally administered antigen may be or comprise a vaccine for a viral infection. For example, the antigen may be a coronavirus vaccine, such as a DNA or RNA vaccine, which may be parenterally administered.

In another embodiment, it is preferred that a dead bacterial spore is used. Preferably, the live or dead spore up-regulates immunity by interacting with a Toll-like receptor, preferably TLR2 and/or TLR4. The inventor believes that this is a likely mechanism for how spores stimulate an innate immune response.

As illustrated in Example 7, the inventors observed that autoclaved bacterial spores enhanced CD4⁺ and γδ T cell recruitment into lung alveolar space during viral infection. The inventors believe that CD4⁺ and γδ T cells may have a regulatory role that ameliorates tissue damage during virus infection. Thus, preferably, the live or dead bacterial spore, the live or dead vegetative bacterium, extracellular material produced by the live cell, or the disrupted bacterial cell homogenate increases recruitment of T cells to the site of viral infection. Even more preferably, the live or dead bacterial spore, the live or dead vegetative bacterium, extracellular material produced by the live cell, or the disrupted bacterial cell homogenate increases recruitment of CD4⁺, CD8⁺ and/or γδ T cells to the site of viral infection.

In addition, as described in Example 7, the inventors surprisingly found that spore pre-treatment reduced natural killer (NK) cell recruitment into the lung alveolar space at five days post H1N1 infection. The inventor believes that exuberant NK cell infiltration may contribute to lung tissue damage in viral pneumonia. Thus, preferably, the live or dead bacterial spore, the live or dead vegetative bacterium, extracellular material produced by the live cell, or the disrupted bacterial cell homogenate reduces natural killer (NK) cell recruitment into the lung following the virus infection.

In yet another embodiment, a live bacterial cell is used to combat the viral infection. In yet a further embodiment, a dead bacterial cell is used to combat the viral infection. The skilled person will appreciate that there are several ways in which the bacterial spore or cell may be killed or rendered non-viable, such as autoclaving, formaldehyde inactivation, irradiation (e.g., Gamma radiation), heating (e.g., pasteurisation), or through thymine synthetase inactivation, as described in the inventor's patent application, WO2019/086887. Pasteurisation is a known technique using mild heat (usually less than about 100° C. in order to kill only vegetative cells, but not spores). Example 6 shows that heat-inactivated spores of Bacillus subtilis can confer effective protection to coronavirus infections when administered via a mucosal route, as demonstrated by 80% survival of mice treated with B. subtilis spores. Accordingly, this demonstrates that spores have the ability to surprisingly protect against SARS-CoV-2 and improve survival rates following coronavirus infections.

The dead cell may be intact. Alternatively, the dead cell may comprise a broken or a disrupted cell, i.e., one that has been mechanically or physically disrupted by, for example, sonication or an enzyme, such as lysozyme etc. In this embodiment, the disrupted cell's integuments, envelope-associated integuments and exopolysaccharides (EPS) etc. would exhibit the antiviral activity. Hence, in a further embodiment, it is preferred that a disrupted cell homogenate is used.

However, in a most preferred embodiment, a live vegetative bacterial cell or extracellular material produced by the live cell, is used to combat the infection. In another preferred embodiment, a cell-free sample (e.g., the supernatant) comprising extracellular material produced by the live vegetative cell or disrupted cell homogenate may be used to combat the viral infection.

Preferably, the bacterium (be that the live or dead spore, or the live or dead vegetative bacterial cell, or the bacterium which produces the extracellular material or cell homogenate) is a spore-forming bacterium belonging to the phyla Firmicutes.

Preferably, the bacterium (be that the live or dead spore, or the live or dead vegetative bacterial cell, or the bacterium which produces the extracellular material or cell homogenate) is a Bacillus spp or Clostridium spp. More preferably, the bacterium is a Bacillus spp.

Preferably, the Bacillus spp is Bacillus subtilis, Bacillus amyloliquefaciens, Bacillus velezensis, Bacillus clausii, Bacillus coagulans, Bacillus pumilus, Bacillusfirmus, Bacillus flexus, Bacillus licheniformis, Bacillus marisflavi, Bacillus polyfermenticus, Bacillus megaterium, Bacillus flexus, or Bacillus indicus.

Preferably, the bacterium is B. subtilis. Preferably, the bacterium is B. amyloliquefaciens or B. velezensis. The inventors believe that B. velezensis is a very close relative of B. amyloliquefaciens, and has recently been shown to be a new species in its own right (Wang, L. T., Lee, F. L., Tai, C. J. & Kuo, H. P. Bacillus velezensis is a later heterotypic synonym of Bacillus amyloliquefaciens. Int J Syst Evol Microbiol 58, 671-675, doi:10.1099/ijs.0.65191-0 (2008).

In another embodiment, the bacteria may be as defined in Table 1, deposited at the DSMZ, InhoffenstraBe 7B, 38124 Braunschweig, Germany:

TABLE 1
Preferred Strains of Bacteria used

| SG name | Species | DSMZ number | Date of deposit |
|---|---|---|---|
| SG154 | B. pumilus | DSM 33465 | 13 Mar. 2020 |
| SG43 | B. pumilus | DSM 32394 | 22 Nov. 2016 |
| SG183 | B. megaterium | DSM 32395 | 22 Nov. 2016 |
| SG188 | B. subtilis | DSM 32444 | 6 Mar. 2017 |
| SG336 | B. subtilis | DSM 32445 | 6 Mar. 2017 |
| SG2404 | B. subtilis var. | DSM 32446 | 6 Mar. 2017 |
| | Natto | | |

Accordingly, preferably, the bacterium may be selected from a group consisting of SG154, SG43, SG183, SG188, SG336 and SG2404, as denoted in Table 1.

Preferably, the B. amyloliquefaciens or B. velezensis strain that is used is selected from a group consisting of: SG57, SG137, SG185, SG277 and SG297. Most preferably, the B. amyloliquefaciens strain is SG277 or SG297. The B. subtilis strain is SG140.

In another embodiment, the bacterium may be as defined in Table 2.

TABLE 2
Preferred Strains of Bacteria used

| Species | Sporegen name | NCIMB^{a} name |
|---|---|---|
| B. subtilis | SG140 | NCIMB 42974 |
| B. velezensis^{b} | SG57 | NCIMB 43392 |
| B. velezensis^{b} | SG137 | NCIMB 43393 |
| B. velezensis^{b} | SG185 | NCIMB 42973 |
| B. velezensis^{b} | SG277 | NCIMB 42971 |
| B. velezensis^{b} | SG297 | NCIMB 42972 |

^{a}originally classified as B. amyloliquefaciens
^{b}NCIMB Ltd (https://www.ncimb.com)

In one embodiment, one or more strains of B. amyloliquefaciens, or extracellular material produced by the cell or disrupted cell homogenate, is used. In other words, any B. amyloliquefaciens strain selected from a group consisting of: SG57, SG137, SG185, SG277 and SG297, may be used. Alternatively, in another embodiment, more than one B. amyloliquefaciens strain selected from a group consisting of: SG57, SG137, SG185, SG277 and SG297, may be used. For example, SG277 and SG297 could be used simultaneously, or SG137 and SG57 could be used simultaneously, and so on.

In yet another embodiment, one or more strains of B. amyloliquefaciens may be used in combination with B. subtilis, or extracellular material produced by the corresponding cell or disrupted cell homogenate therefrom. For example, B. amyloliquefaciens strain SG277 may be used with B. subtilis strain SG140.

It will be appreciated that any of the bacterial strains described herein can be used as the live or dead spore, or the live or dead vegetative bacterial cell, or the bacterium which produces the extracellular material or cell homogenate.

The most preferred strains are strains deposited under the Budapest Treaty at the NCIMB, Ferguson Building, Craibstone Estate, Bucksburn, Aberdeen, AB21 9YA on 15 Feb. 2018 and 10 May 2019, as follows:

Designation number: NCIMB 42971—Referred to herein as: B. amyloliquefaciens SG277.

Designation number: NCIMB 42972—Referred to herein as: B. amyloliquefaciens SG297.

Designation number: NCIMB 42973—Referred to herein as: B. amyloliquefaciens SG185.

Designation number: NCIMB 42974—Referred to herein as: B. subtilis SG140.

Designation number: NCIMB 43392—Referred to herein as B. amyloliquefaciens SG57.

Designation number: NCIMB 43393—Referred to herein as B. amyloliquefaciens SG137.

Recent changes in the bacterial taxonomy include the reclassification of B. amyloliquefaciens strains as B. velezensis (Wang et al, 2008; Fan, B., Blom, J., Klenk, H. P. & Borriss, R. Bacillus amyloliquefaciens, Bacillus velezensis, and Bacillus siamensis Form an “Operational Group B. amyloliquefaciens” within the B. subtilis Species Complex. Front Microbiol 8, 22, doi:10.3389/fmicb.2017.00022 (2017)). The present application discloses strains designated SG57, SG137, SG185, SG277 and SG297, and these strains have been designated B. amyloliquefaciens without taking recent changes in the taxonomy into account. It should therefore be understood that the species designation B. amyloliquefaciens as used in the present description and claims includes strains that a taxonomy expert would designate as B. velezensis strains.

In yet another embodiment, one or more strains of B. amyloliquefaciens may be used in combination with one or more strains of B. subtilis, or extracellular material produced by the corresponding cell or disrupted cell homogenate therefrom. For example two strains, B. amyloliquefaciens strain NCIMB 42971 may be used with B. amyloliquefaciens NCIMB 42972, B. amyloliquefaciens NCIMB 42973, B. subtilis NCIMB 42974, B. amyloliquefaciens NCIMB 43392 or B. amyloliquefaciens NCIMB 43393; B. amyloliquefaciens strain NCIMB 42972 may be used with B. amyloliquefaciens NCIMB 42973, B. subtilis NCIMB 42974, B. amyloliquefaciens NCIMB 43392 or B. amyloliquefaciens NCIMB 43393; B. amyloliquefaciens strain NCIMB 42973 may be used with B. subtilis NCIMB 42974, B. amyloliquefaciens NCIMB 43392 or B. amyloliquefaciens NCIMB 43393; B. subtilis NCIMB 42974 may be used with B. amyloliquefaciens NCIMB 43392 or B. amyloliquefaciens NCIMB 43393; or B. amyloliquefaciens NCIMB 43392 may be used with B. amyloliquefaciens NCIMB 43393.

The bacterium may be a B. amyloliquefaciens strain, and the B. amyloliquefaciens strain is selected from the strains deposited as NCIMB 42971, NCIMB 42972, NCIMB 42973, NCIMB 43392 or NCIMB 43393. The bacterium may comprise a B. subtilis strain, and the B. subtilis strain is the strain deposited as NCIMB 42974. All of the bacteria may be selected from the B. amyloliquefaciens strains deposited as NCIMB 42971, NCIMB 42972, NCIMB 42973, NCIMB 43392 or NCIMB 43393; and the B. subtilis strain deposited as NCIMB 42974.

Preferably, the virus is a respiratory virus. Preferably, the virus is selected from the group consisting of: Respiratory syncytial virus (RSV), Coronavirus and Rhinovirus.

Preferably, the respiratory virus is a Coronavirus. More preferably, the Coronavirus is selected from MERS, SARS-CoV1 and SARS-CoV2. Most preferably, the respiratory virus is SARS-CoV2. It will be appreciated that SARS-CoV2 is the causative agent of COVID-19.

Preferably, the use comprises mucosal application, to a subject, of the live or dead bacterial spore, live or dead vegetative bacterium, extracellular material produced by the live cell, or disrupted bacterial cell homogenate. Mucosal application may comprise nasal, rectal, ocular, oral or sub-lingual application of the live or dead bacterial spore, live or dead vegetative bacterium, extracellular material produced by the live cell, or disrupted bacterial cell homogenate.

Preferably, the live or dead bacterial spore, live or dead vegetative bacterium, extracellular material produced by the live cell, or disrupted bacterial cell homogenate for use in treating the viral infection, is applied parenterally. As such, the inventor believes that injected bacterial spores, which are preferably dead, may be used as an effective vaccine adjuvant. Preferably, the live or dead bacterial spore, live or dead vegetative bacterium, extracellular material produced by the live cell, or disrupted bacterial cell homogenate for use in treating the viral infection, is applied nasally. Nasal application may comprise application by a spray or droplet.

Preferably, the live or dead bacterial spore, live or dead vegetative bacterium, extracellular material produced by the live cell, or disrupted bacterial cell homogenate is applied sublingually, which the skilled person would understand relates to the slow release of molecules in the oral cavity (more specifically, under the tongue). This approach is advantageous in that is avoids the gastro-intestinal route and potential issues over tolerance. Moreover, the sublingual route enables interaction with the sublingual lymphoid glands. Sublingual application may comprise application by a wafer (e.g., a buccal wafer) or fast-dissolving film.

In one embodiment, it is preferred that a live bacterial spore is used to treat a coronavirus infection, preferably SARS-CoV2 infection, and is applied sublingually, preferably by wafer (e.g., a buccal wafer) or a fast dissolving film. In one embodiment, it is preferred that a dead bacterial spore is used to treat a coronavirus infection, preferably SARS-CoV2 infection, and is applied sublingually, preferably by wafer (e.g., a buccal wafer) or fast-dissolving film.

In another embodiment, it is preferred that a live bacterial spore is used to treat a coronavirus infection, preferably SARS-CoV2 infection, and is applied nasally, preferably by spray or droplet. In one embodiment, it is preferred that a dead bacterial spore is used to treat a coronavirus infection, preferably SARS-CoV2 infection, and is applied nasally, preferably by spray or droplet.

For example, in one embodiment, there is provided a dead B. subtilis spore, for use in treating, preventing or ameliorating a SARS-CoV-2 infection, wherein the dead B. subtilis spore is applied nasally.

In one embodiment, it is preferred that a live bacterial spore is used to treat a coronavirus infection, preferably SARS-CoV2 infection, and is applied parenterally. In one embodiment, it is preferred that a dead bacterial spore is used to treat a coronavirus infection, preferably SARS-CoV2 infection, and is applied parenterally.

As shown in Example 2, the inventor believes that they have identified one of the salient factors that is integral for the protective effect of the spore-forming bacteria against viral infections by induction of the innate immune system. These are heptaprenyl lipids termed “sporulenes”, which are believed to possess adjuvant activity, possibly by activating TLR2 and/or TLR4. The squalene cyclase gene, sqhC, encodes the squalene cyclase enzyme (a Sporulenol synthase) that bacterial spores utilise to produce sporulenes.

Thus, preferably, the bacterium comprises the squalene cyclase gene, sqhC, which may be represented by NCBI GeneID number 939443, or a homologue, orthologue or equivalent thereof, and/or wherein the bacterium comprises one or more sporulene. In one embodiment, the open reading frame of sqhC may be encoded by the nucleic acid provided herein as SEQ ID No: 28, as follows:

[SEQ ID No: 28]
ATGGGCACACTTCAGGAGAAAGTGAGGCGTTTTCAAAAGAAAACCATTA
CCGAGTTAAGAGACAGGCAAAATGCTGATGGTTCATGGACATTTTGCTT
TGAAGGACCAATCATGACAAATTCCTTTTTTATTTTGCTCCTTACCTCA
CTAGATGAAGGCGAAAATGAAAAAGAACTGATATCATCCCTTGCAGCCG
GCATTCATGCAAAACAGCAGCCAGACGGCACATTTATCAACTATCCCGA
TGAAACGCGCGGAAATCTAACGGCTACCGTCCAAGGATATGTCGGGATG
CTGGCTTCAGGATGTTTTCACAGAACTGAGCCGCACATGAAGAAAGCTG
AACAATTTATCATCTCACATGGCGGTTTGAGACATGTTCATTTTATGAC
AAAATGGATGCTTGCCGCGAACGGGCTTTATCCTTGGCCTGCTTTGTAT
TTACCATTATCACTCATGGCGCTCCCCCCAACATTGCCGATTCATTTCT
ATCAGTTCAGCTCATATGCCCGTATTCATTTTGCTCCTATGGCTGTAAC
ACTCAATCAGCGATTTGTCCTTATTAACCGCAATATTTCATCTCTTCAC
CATCTCGATCCGCACATGACAAAAAATCCTTTCACTTGGCTTCGGTCTG
ATGCTTTCGAAGAAAGAGATCTCACGTCTATTTTGTTACATTGGAAACG
CGTTTTTCATGCACCATTTGCTTTTCAGCAGCTGGGCCTACAGACAGCT
AAAACGTATATGCTGGACCGGATTGAAAAAGATGGAACATTATACAGCT
ATGCGAGCGCAACCATATATATGGTTTACAGCCTTCTGTCACTTGGTGT
GTCACGCTATTCTCCTATTATCAGGAGGGCGATTACCGGCATTAAATCA
CTGGTGACTAAATGCAACGGGATTCCTTATCTGGAAAACTCTACTTCAA
CTGTTTGGGATACAGCTTTAATAAGCTATGCCCTTCAAAAAAATGGTGT
GACCGAAACGGATGGCTCTGTTACAAAAGCAGCCGACTTTTTGCTAGAA
CGCCAGCATACCAAAATAGCAGATTGGTCTGTCAAAAATCCAAATTCAG
TTCCTGGCGGCTGGGGGTTTTCAAACATTAATACAAATAACCCTGACTG
TGACGACACTACAGCCGTTTTAAAGGCGATTCCCCGCAATCATTCTCCT
GCAGCATGGGAGCGGGGGGTATCTTGGCTTTTATCGATGCAAAACAATG
ACGGCGGATTTTCTGCTTTCGAAAAAAATGTGAACCATCCACTGATCCG
CCTTCTGCCGCTTGAATCCGCCGAGGACGCTGCAGTTGACCCTTCAACC
GCCGACCTCACCGGACGTGTACTGCACTTTTTAGGCGAGAAAGTTGGCT
TCACAGAAAAACATCAACATATTCAACGCGCAGTGAAGTGGCTTTTCGA
ACATCAGGAACAAAATGGGTCTTGGTACGGCAGATGGGGTGTTTGCTAC
ATTTACGGCACTTGGGCTGCTCTTACTGGTATGCATGCATGCGGGGTTG
ACCGAAAGCATCCCGGTATACAAAAGGCTCTGCGTTGGCTCAAATCCAT
ACAAAATGATGACGGAAGCTGGGGAGAATCCTGCAAAAGCGCCGAAATC
AAAACATATGTACCGCTTCATAGAGGAACCATTGTACAAACGGCCTGGG
CTTTAGACGCTTTGCTCACATATGAAAATTCCGAACATCCGTCTGTTGT
GAAAGGCATGCAATACCTTACCGACAGCAGTTCGCATAGCGCCGATAGC
CTCGCGTATCCAGCAGGGATCGGATTGCCGAAGCAATTTTATATTCGCT
ATCACAGTTATCCATATGTATTCTCTTTGCTGGCTGTCGGGAAGTATTT
AGATTCTATTGAAAAGGAGACAGCAAATGAAACGTGA

Accordingly, preferably the bacterium comprises a nucleic acid sequence comprising or consisting of the nucleic acid substantially as set out in SEQ ID No: 28, or a fragment or variant thereof.

Genome searching has shown that a SqhC gene is present in Bacillus and Clostridia. Accordingly, other Bacilli and Clostridia will also have SqhC homologues or orthologues or SqhC equivalents, and thus it would be expected that other spore-forming bacteria will also convey innate immunity protection against respiratory virus infection, based on the inventor's work.

Preferably, the live or dead bacterial spore, live or dead vegetative bacterium, extracellular material produced by the live cell, or disrupted bacterial cell homogenate produces or comprises a sporulene family member. The sporulene family member may have a structure as set out in formula VII, as follows:

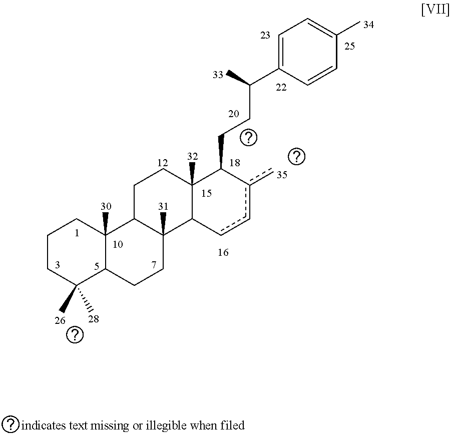

In one embodiment, the sporulene family member is selected from the group consisting of sporulene A, B and C.

In one embodiment, the sporulene family member comprises sporulene A, or an active derivative thereof. In one embodiment, the sporulene family member comprises sporulene B, or an active derivative thereof. In one embodiment, the sporulene family member comprises sporulene C, or an active derivative thereof.

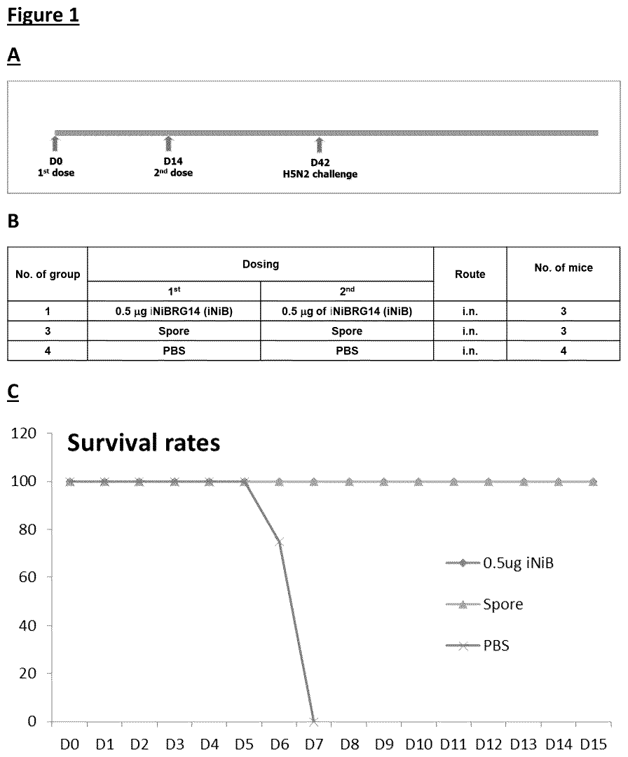

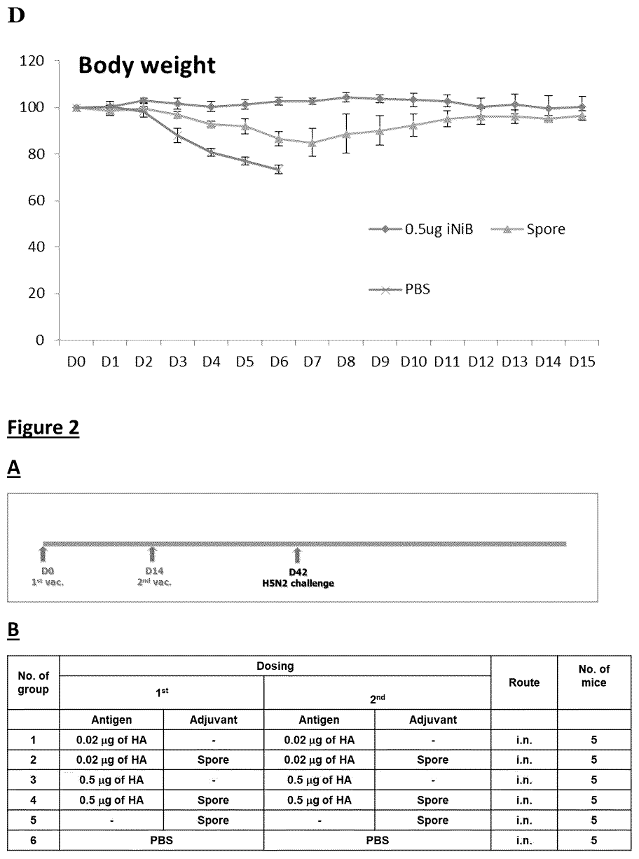

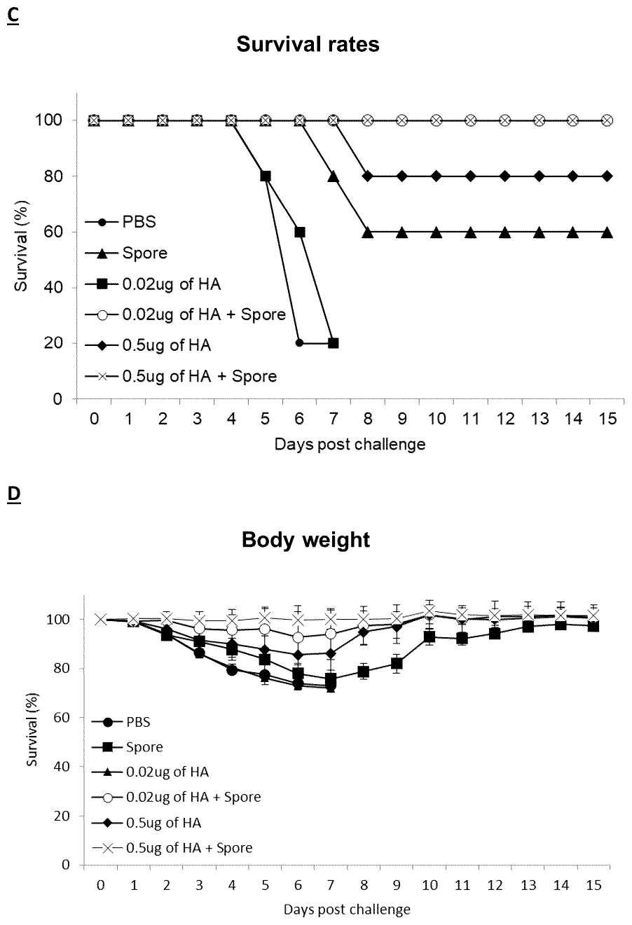

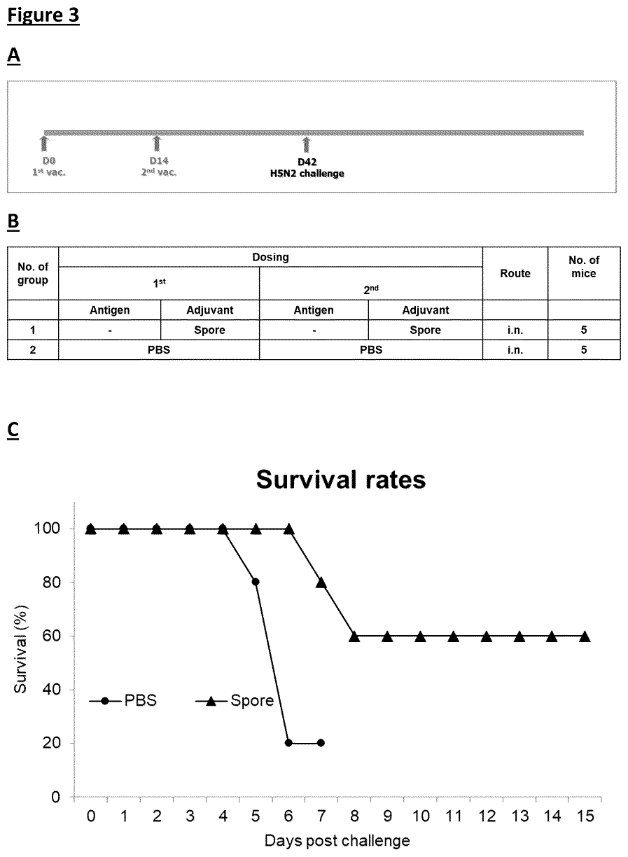

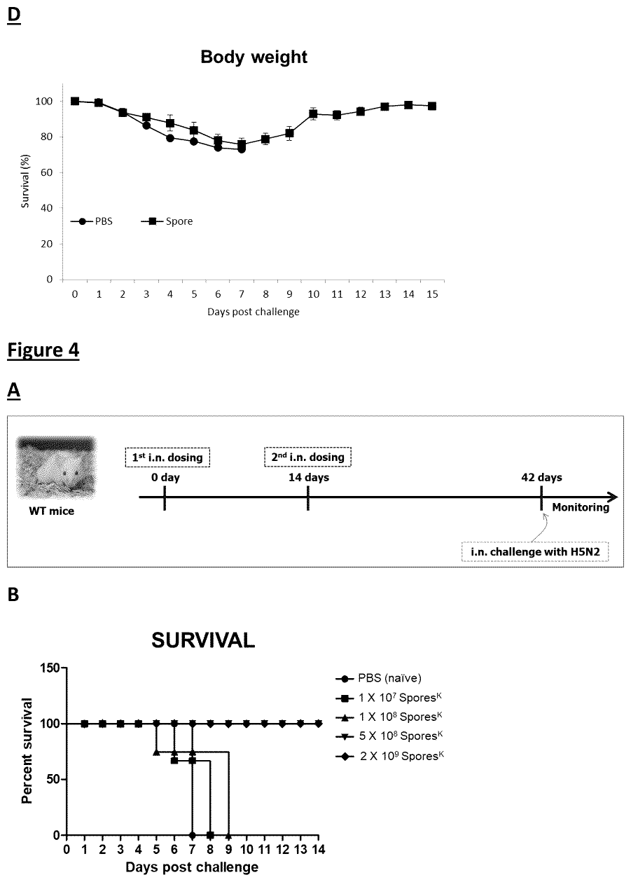

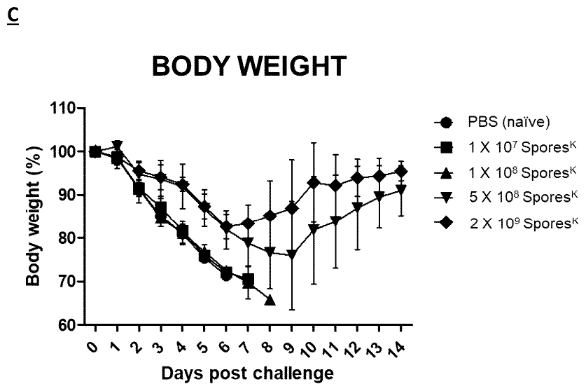

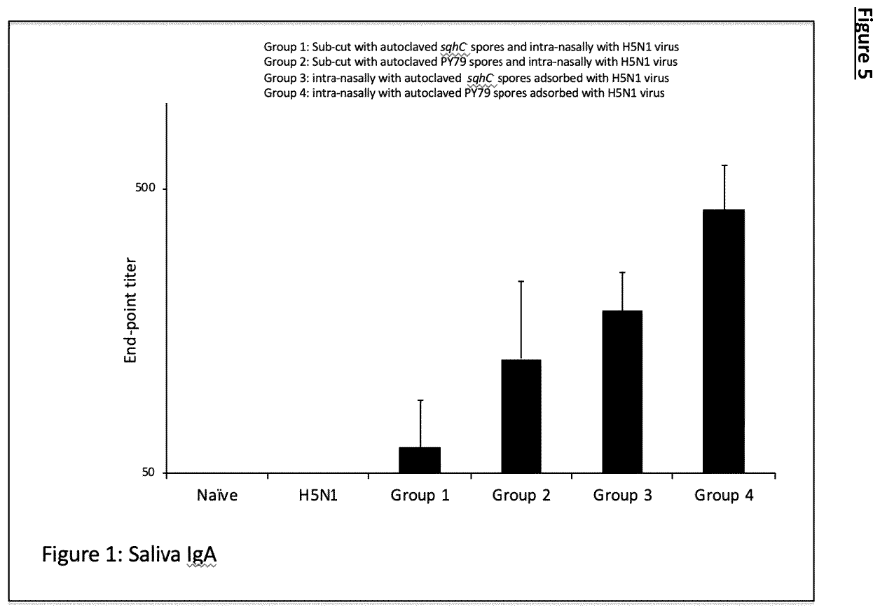

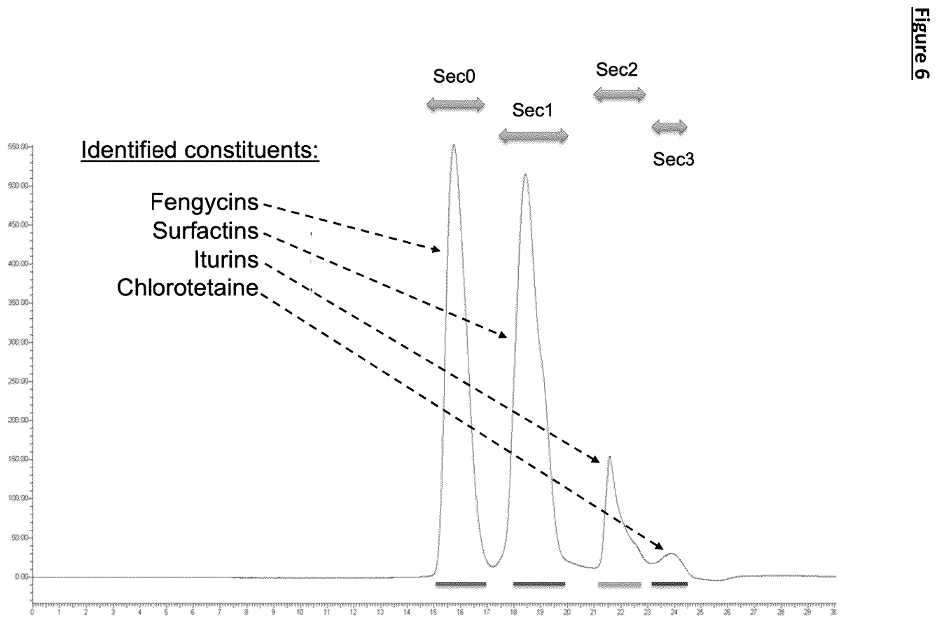

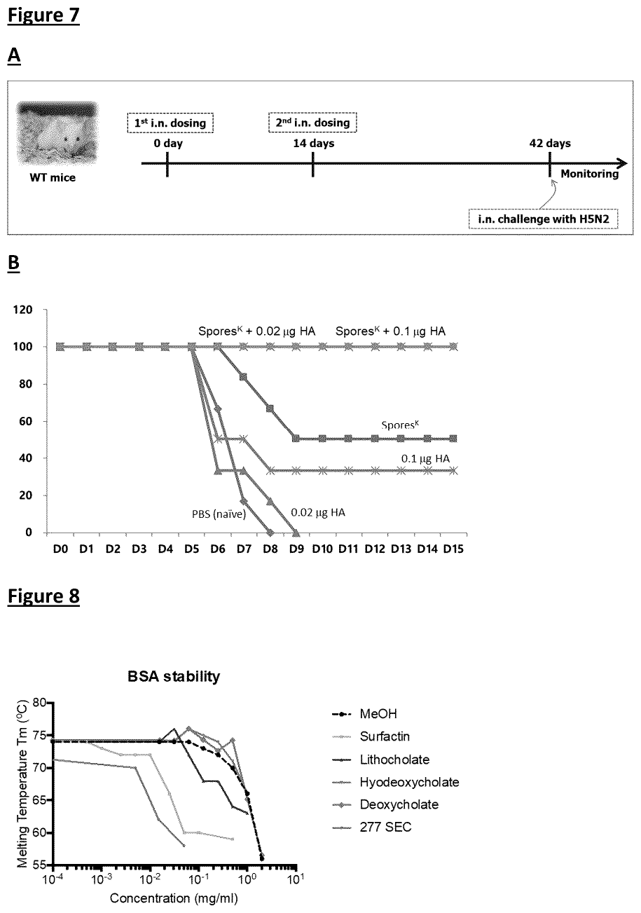

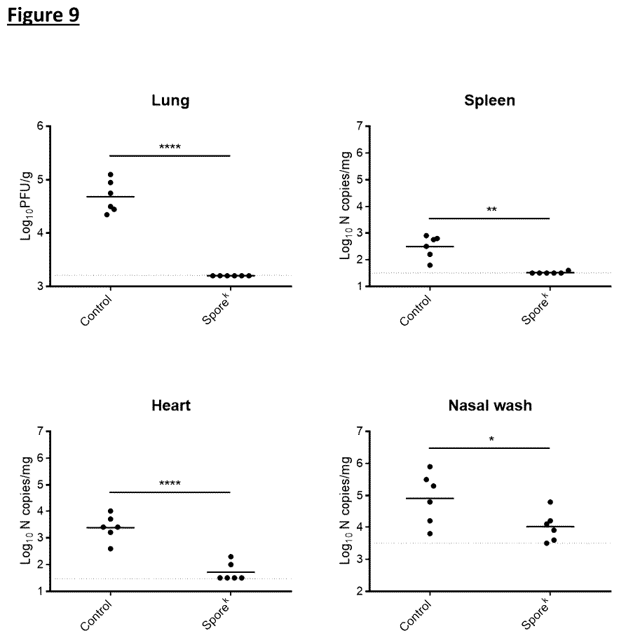

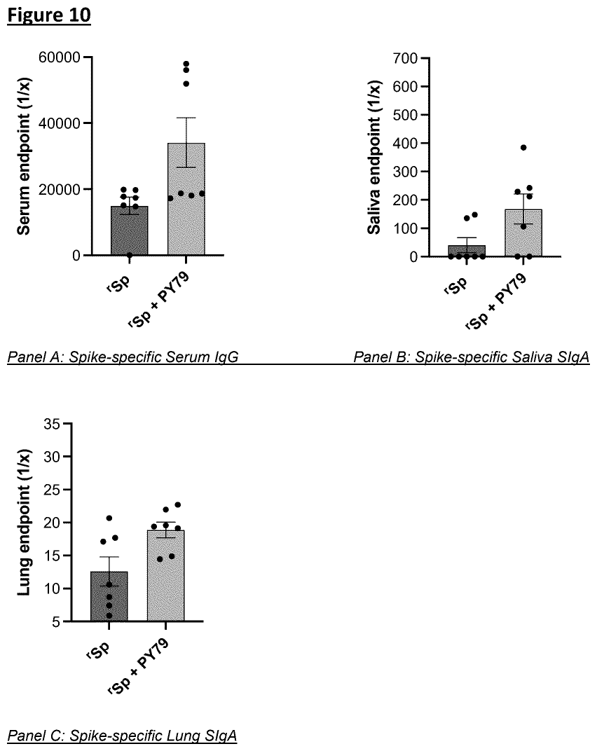

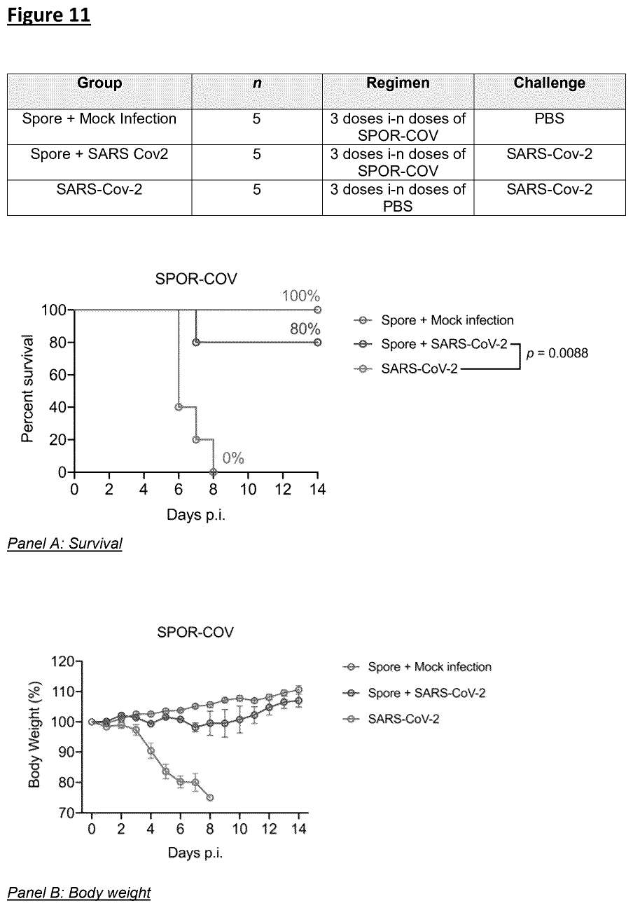

Sporulenes A, B and C are believed to be derived from C35-terpenes, via the enzyme squalene cyclase, as shown in FIG. 1 of Takigawa H, Sugiyama M, Shibuya Y. C(35)-terpenes from Bacillus subtilis KSM 6-10. J Nat Prod. 2010; 73(2):204-7; doi: 10.1021/np900705q.

Thus, in one embodiment, sporulene A may have a structure as set out in formula VIII, as follows:

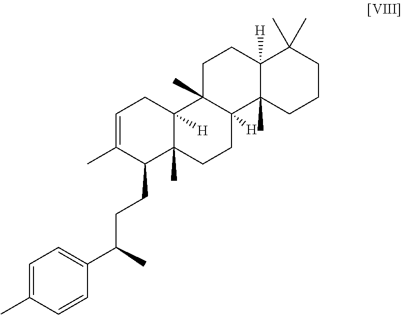

In one embodiment, sporulene B may have a structure as set out in formula IX, as follows:

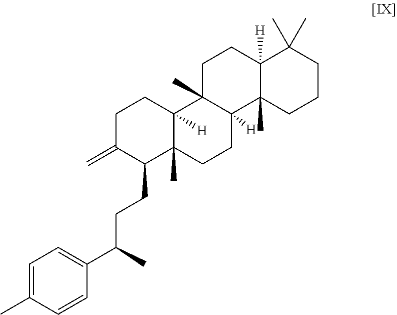

In one embodiment, sporulene C may have a structure as set out in formula X, as follows:

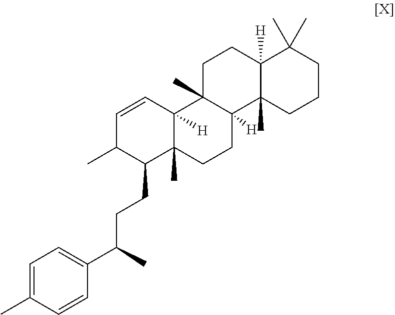

Thus, the live or dead bacterial spore, live or dead vegetative bacterium, extracellular material produced by the live cell, or disrupted bacterial cell homogenate may produce or comprise at least one sporulene family member. Preferably, the live or dead bacterial spore, live or dead vegetative bacterium, extracellular material produced by the live cell, or disrupted bacterial cell homogenate may produce or comprise a sporulene so family member selected from the group consisting of: sporulene A, sporulene B and sporulene C.

Preferably, the live or dead bacterial spore, live or dead vegetative bacterium, extracellular material produced by the live cell, or disrupted bacterial cell homogenate may produce or comprise sporulene A, sporulene B and sporulene C. Preferably, the live or dead bacterial spore, live or dead vegetative bacterium, extracellular material produced by the live cell, or disrupted bacterial cell homogenate may produce or comprise sporulene A and sporulene B. Preferably, the live or dead bacterial spore, live or dead vegetative bacterium, extracellular material produced by the live cell, or disrupted bacterial cell homogenate may produce or comprise sporulene A and sporulene C. Preferably, the live or dead bacterial spore, live or dead vegetative bacterium, extracellular material produced by the live cell, or disrupted bacterial cell homogenate may produce or comprise sporulene B and sporulene C.

The inventor has previously identified factors produced by Bacillus species, as shown in FIG. 8, that display antibacterial properties (as described in WO2019/16252) and which can be used in compositions to treat Clostridium difficile infections. The inventor now believes that these factors also surprisingly display antiviral activity, and can therefore provide an additional means by which the live or dead bacterial spore, live or dead vegetative bacterium, extracellular material produced by the live cell, or disrupted bacterial cell homogenate may be used to treat, prevent or ameliorate a virus infection, such as COVID-19, by direct inhibition of the virus. This activity is different to stimulation of the innate immune system as described above and is believed to result from the ability of these molecules to denature viral envelope/capsid proteins. The inventor has shown that these molecules attach to the surface of spores (either dead or alive). Accordingly, the inventor believes that this dual effect of eliciting an innate immune response in addition to directly inhibiting the virus itself helps create a highly robust vaccination or prophylactic immune response.

Additionally, these factors (e.g., lipopeptides) have also been shown to display adjuvant properties [15-18], which may or may not also be involved with the innate immune response. Thus, it should be appreciated that the live or dead bacterial spore, live or dead vegetative bacterium, extracellular material produced by the live cell, or disrupted bacterial cell homogenate has: (i) anti-viral properties, and (ii) adjuvant properties, which may influence the innate immunity.

Thus, in one embodiment, the live or dead bacterial spore, live or dead vegetative bacterium, the extracellular material produced by the live cell or the disrupted bacterial cell homogenate produces or comprises one or more non-ribosomal peptide.

As used herein, the term “non-ribosomal peptide” (also known as non-ribosomal peptides or NRP) means a class of peptide secondary metabolites that are synthesized by non-ribosomal peptide synthetases. Preferably, the one or more non-ribosomal peptides of the invention are lipopeptides.

Non-ribosomal peptides of the invention include, but are not limited to, lipopeptides that are members of the Fengycin family, members of the Surfactin family, and members of the Iturin family.

The non-ribosomal peptides may be one or more peptides selected from the group consisting of a member of the Fengycin family; a member of the Surfactin family; and a member of the Iturin family. Preferably, the non-ribosomal peptides may be one or more peptides selected from the group consisting of: a member of the Fengycin family; and a member of the Surfactin family. Most preferably, the live or dead bacterial spore, live or dead vegetative bacterium, the extracellular material produced by the live cell or the disrupted bacterial cell homogenate may produce or comprise the non-ribosomal peptides: a member of the Fengycin family, and a member of the Surfactin family.

The non-ribosomal peptides may be one or more peptides selected from the group consisting of a member of the Fengycin family; a member of the Surfactin family; a member of the Iturin family. Preferably, the non-ribosomal peptides may be a member of the Fengycin family and a member of the Surfactin family.

The live or dead bacterial spore, live or dead vegetative bacterium, the extracellular material produced by the live cell or the disrupted bacterial cell homogenate may produce or comprise two or more non-ribosomal peptides. The two or more non-ribosomal peptides may be selected from the group consisting of: a member of the Fengycin family; a member of the Surfactin family; and a member of the Iturin family. The two or more non-ribosomal peptides may be selected from the group consisting of: a member of the Fengycin family; and a member of the Surfactin family. The live or dead bacterial spore, live or dead vegetative bacterium, the extracellular material produced by the live cell or the disrupted bacterial cell homogenate may produce or comprise three or more non-ribosomal peptides. Preferably, the non-ribosomal peptides are: a member of the Fengycin family; and a member of the Surfactin family.

In one embodiment, the general formula for members of the Fengycin family may be set out in formula I below, wherein R1 to R3 is any amino acid, and preferably R1 is L or D Tyr, R2 is Ala or Val and R3 is L or D Tyr:

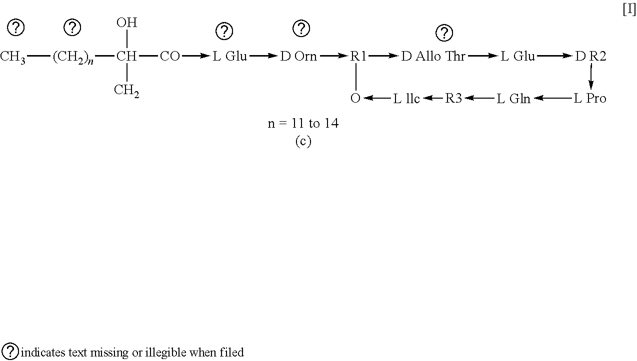

The member of the Fengycin family may be selected from a group consisting of: Fengycin A [SEQ ID NO: 11], Fengycin B [SEQ ID NO: 12], Plipastatin A [SEQ ID NO: 13] and Plipastatin B [SEQ ID NO: 14].

| | | |
|---|---|---|
| Fengycin A** | L-Glu-D-Orn-D-Tyr-D- | [symbol] |
| | aThr-L-Glu-D-Ala-L- | |
| | Pro-L-Gln-L-Tyr-L-Ile | |
| Fengycin B** | L-Glu-D-Orn-D-Tyr-D- | [symbol] |
| | aThr-L-Glu-D-Val-L- | |
| | Pro-L-Gln-L-Tyr-L-Ile | |
| Plipastatin A | L-Glu-D-Orn-L-Tyr-D- | [symbol] |
| | aThr-L-Glu-D-Ala-L- | |
| | Pro-L-Gln-D-Tyr-L-Ile | |
| Plipastatin B | L-Glu-D-Orn-L-Tyr-D- | [symbol] |
| | aThr-L-Glu-D-Val-L- | |
| | Pro-L-Gln-D-Tyr-L-Ile | |

[symbol]

indicates data missing or illegible when filed

Preferably, the member of the Fengycin family comprises Fengycin A, or an active derivative thereof. The Fengycin A, or active derivative thereof, may be the C₁₅, C₁₆, C₁₇ or C₁₈ isoform. Most preferably, the Fengycin A is the C₁₅ Fengycin A isoform. The Fengycin A, or active derivative thereof, may be acetylated.

In one embodiment, Fengycin A may have an amino acid sequence as set out in SEQ ID NO: 11:

[SEQ ID NO 11]
L-Glu-D-Orn-D-Tyr-D-aThr-L-Glu-D-Ala-L-Pro-L-
Gln-L-Tyr-L-Ile

Preferably, the member of the Fengycin family comprises Fengycin B, or an active derivative thereof. The Fengycin B, or active derivative thereof, may be the C₁₃, C₁₄, C₁₅ or C₁₆ isoform. Most preferably, the Fengycin B is the C₁₅ Fengycin B isoform. The Fengycin B, or active derivative thereof, may be acetylated.

In one embodiment, Fengycin B may have an amino acid sequence as set out in SEQ ID NO: 12:

[SEQ ID NO: 12]
L-Glu-D-Orn-D-Tyr-D-aThr-L-Glu-D-Val-L-Pro-L-
Gln-L-Tyr-L-ILe

In one embodiment where the peptide is a member of the Surfactin family, the general formula for the members of the Surfactin family may be set out in formula II below, wherein R1-R4 is any amino acid, and preferably R1 is glutamine or glutamic acid, R2 is leucine or valine, R3 is valine, leucine or alanine, and R4 is leucine or valine.

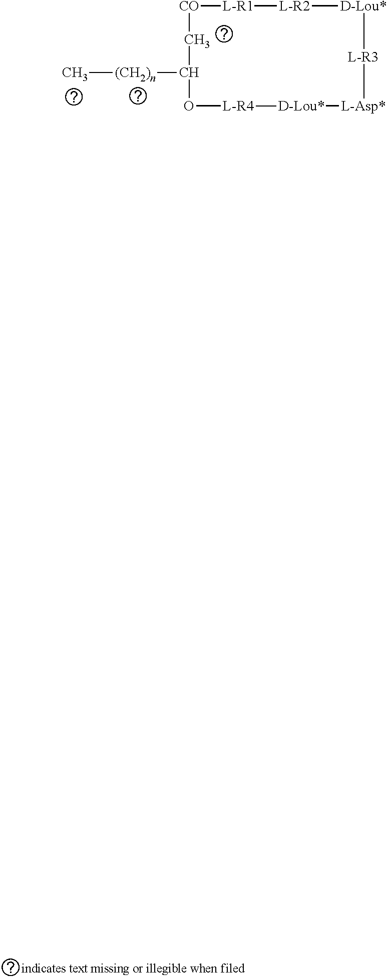

The member of the Surfactin family may be selected from a group consisting of: Esperin [SEQ ID NO: 15], Lichenysin [SEQ ID NO: 16], Pumilacidin [SEQ ID NO: 17] and Surfactin [SEQ ID NO: 18].

| | | |
|---|---|---|
| Esperin | L-Glu-L-Leu-D-Leu-L-Val-L- | |
| | Asp-D-Leu-L-Leu-COOH | |
| Lichenysin | L-XL₁-L-XL₂-D-Leu-L-XL₄-L- | [symbol] |
| | Asp-D-Leu-L-XL₇ | |
| Pumilacidin | L-Glu-L-Leu-D-Leu-L-Leu-L- | |
| | Asp-D-Leu-L-XP₇ | |
| Surfactin | L-Glu-L-XS₂-D-Leu-L-XS₄-L- | [symbol] |
| | Asp-D-Leu-L-XS₇ | |

[symbol]

indicates data missing or illegible when filed

Wherein XL1 is Gln or Glu; XL2 is Leu or Ile; XL4 and XL7 are Val or ILe XP7 is Val or Ile XS2 is Val, Leu or ILe; XS4 is Ala, Val, Leu or ILe; XS7 is Val Leu or Ile.

Preferably, the member of the Surfactin family is Surfactin, or an active derivative thereof. In one embodiment, Surfactin may have an amino acid sequence as set out in SEQ ID NO: 18:

[SEQ ID NO: 18]
L-Glu-L-XS₂-D-Leu-L-XS₄-L-ASP-D-Leu-L-XS₇

Active derivatives of Surfactin may therefore comprise any of the C₁₂, C₁₃, C₁₄, C₁₅, C₁₆, or C₁₇ isoforms. Preferably, the Surfactin is the C₁₆ isoform. The Surfactin, or active derivative thereof, may be the C₁₂, C₁₃, C₁₄, C₁₅, C₁₆, or C₁₇ isoform. Most preferably, the Surfactin, or active derivative thereof, is the C₁₅ isoform.

In one embodiment, a Surfactin may have a structure as set out in formula III:

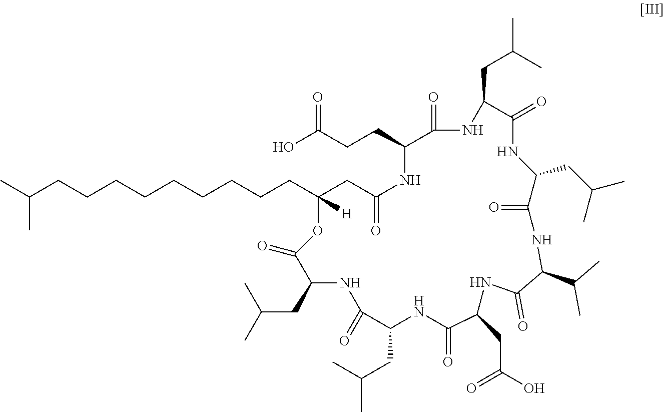 [SMILES: CC(C)CCCCCCCCC[C@@H]1CC(=O)N[C@@H](CCC(=O)O)C(=O)N[C@@H](CC(C)C)C(=O)N[C@H](CC(C)C)C(=O)N[C@@H](C(C)C)C(=O)N[C@@H](CC(=O)O)C(=O)N[C@H](CC(C)C)C(=O)N[C@@H](CC(C)C)C(=O)O1]

In one embodiment, the member of the Iturin family, or active derivative thereof may be selected from a group consisting of: Iturin A, Iturin A_{L}, Iturin C, Mycosubtilin, Bacillomycin D, Bacillomycin F, Bacillomycin L, Bacillomycin LC and Bacillopeptin.

In one embodiment, the general formula for members of the Iturin family or active derivative thereof may be as set out in formula IV below, wherein R1 to R5 is any amino acid, and preferably R1 is Asn or Asp, R2 is Pro, Gln or Ser, R3 is Glu, Pro or Gln, R4 is Ser or Asn and R5 is Thr, Ser or Asn:

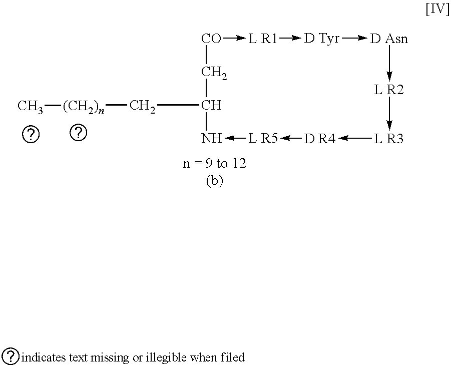

The member of the Iturin family, or active derivative thereof, may be Iturin A [SEQ ID NO:19], Iturin A_{L} [SEQ ID NO:20], Iturin C [SEQ ID NO:21], Mycosubtilin [SEQ ID NO:22], or Bacillomycin D [SEQ ID NO:23], Bacillomycin F [SEQ ID NO:24], Bacillomycin L [SEQ ID NO:25], Bacillomycin LC [SEQ ID NO:26], Bacillopeptin A, Bacillopeptin B or Bacillopeptin C [SEQ ID NO: 27] the sequences of which are shown below:

| | | |
|---|---|---|
| Bacillomycin D | L-Asn-D-Tyr-D-Asn-L-Pro- | [symbol] |
| | L-Glu-D-Ser-L-Thr | |
| Bacillomycin F | L-Asn-D-Tyr-D-Asn-L-Gln- | [symbol] |
| | L-Pro-D-Asn-L-Thr | |
| Bacillomycin L | L-Asn-D-Tyr-D-Asn-L-Ser- | [symbol] |
| | L-Gln-D-Ser-L-Thr | |
| Bacillomycin LC* | L-Asn-D-Tyr-D-Asn-L-Ser- | [symbol] |
| | L-Glu-D-Ser-L-Thr | |
| Iturin A | L-Asn-D-Tyr-D-Asn-L-Gln- | [symbol] |
| | L-Pro-D-Asn-L-Ser | |
| Iturin A_{L} | L-Asn-D-Tyr-D-Asn-L-Gln- | [symbol] |
| | L-Pro-D-Asn-L-Ser | |
| Iturin C | L-Asn-D-Tyr-D-Asn-L-Gln- | [symbol] |
| | L-Pro-D-Asn-L-Ser | |
| Mycosubtilin | L-Asn-D-Tyr-D-Asn-L-Gln- | [symbol] |
| | L-Pro-D-Ser-L-Asn | |

Bacillopeptin A, B and C:
[SEQ ID NO: 27]
cyclo[D-Asn-Ser-Glu-D-Ser-Thr-βAA-Asn-D-Tyr] [symbol]
indicates data missing or illegible when filed

wherein the βAA for each of Bacillopeptin A, B and C is set out under R in Formula V below:

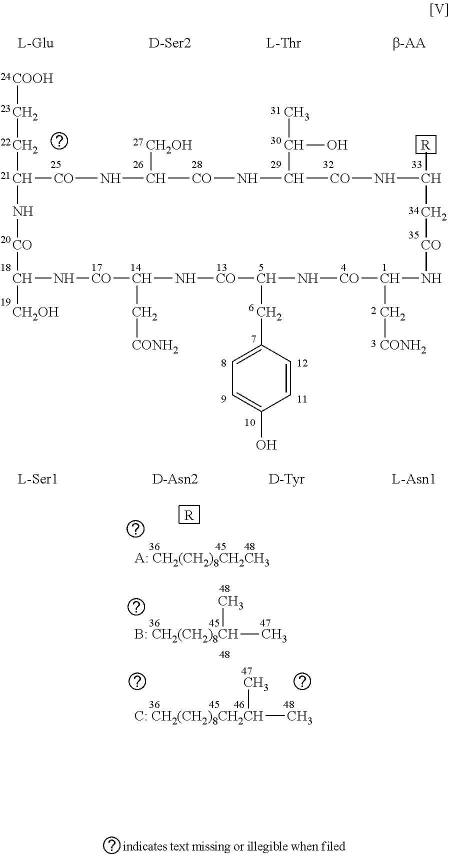

Preferably, the member of the Iturin family is Iturin A, or active derivative thereof. It will be appreciated that Iturin A is a lipopeptide. The Iturin A, or active derivative thereof, may be the C₁₄, C₁₅ or C₁₆ isoform. Active derivatives of Iturin A may therefore comprise any of the C₁₄, C₁₅ or C₁₆ isoforms. Most preferably, the Iturin A, or active derivative thereof, is the C₁₅ Iturin isoform.

In one embodiment, Iturin A may have an amino acid sequence as set out in SEQ ID NO: 19:

[SEQ ID NO: 19]
L-Asn-D-Tyr-D-Asn-L-Gln-L-Pro-D-Asn-L-Ser

Wherein n-C₁₄, i-C₁₅, ai-C₁₅

The inventors also believe that the presence of another biosurfactant, such as a glycolipid, may be particularly advantageous.

Thus, in a preferred embodiment, the live or dead bacterial spore, live or dead vegetative bacterium, the extracellular material produced by the live cell or the disrupted bacterial cell homogenate further produces or comprises a glycolipid.

Preferably, the glycolipid is a Rhamnolipid or an active derivative thereof, and/or a Sophorolipid or an active derivative thereof.

Preferably, the glycolipid is a Rhamnolipid. The Rhamnolipid may be a Mono or Di Rhamnolipid.

Preferably, the Rhamnolipid is selected from the group consisting of the C₈, C_{8:2}, C₁₀, C₁₂, C_{12:2}, C₁₄ or C_{14:2} isoforms.

Preferably, the Rhamnolipid is the C₁₂ isoform.

In one embodiment, the Rhamnolipid has a general formula as set out in formula VI:

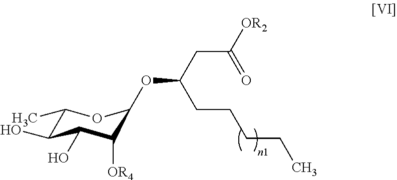

wherein R₁R₂ and n₁ are as set out in the table below for each isoform:

| No. | Symbol | M. Form. | MW | R₁ | n₁ | n₂ | R₂ |
|---|---|---|---|---|---|---|---|
| 1 | Rha-C_{8:2} | C₁₄H₂₂O₇ | 302.32 | H | 1(−4H) | — | H |
| 2 | Rha-C₈ | C₁₄H₂₈O₇ | 306.35 | H | 1 | — | H |
| 3 | Rha-C₁₀ | C₁₈H₃₀O₇ | 334.41 | H | 3 | — | H |
| 4 | Rha-C_{12:2} | C₁₈H₃₀O₇ | 358.43 | H | 5(−4H) | — | H |
| 5 | Rha-C₁₂ | C₁₈H₃₄O₇ | 362.46 | H | 5 | | H |
| 6 | Rha-C_{14:2} | C₂₀H₃₄O₇ | 386.48 | H | 7(−4H) | — | H |

In one embodiment, the Sophorolipid has a general formula as set out in formula XI:

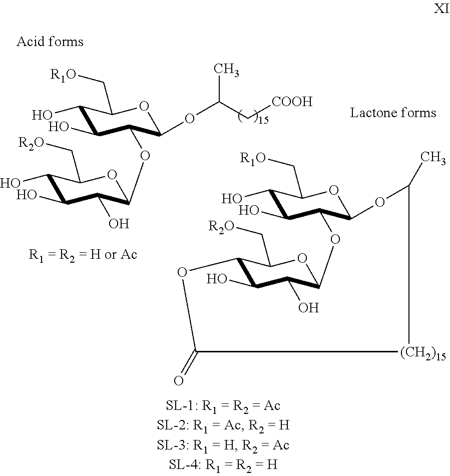

The inventors believe that a composition comprising the combination of lipopeptides and glycolipids is antiviral.

Preferably, the live or dead bacterial spore, live or dead vegetative bacterium, the extracellular material produced by the live cell or the disrupted bacterial cell homogenate according to the invention further produces or comprises a lipopeptide selected from a group consisting of: Mycosubtilin; Mojavensin A; and Kurstakin, or an active derivative of any of these lipopeptides.

The Mycosubtilin, or active derivative thereof, may be the C₁₇ isoform. In one embodiment, Mycosubtilin may have a structure as set out in formula XII:

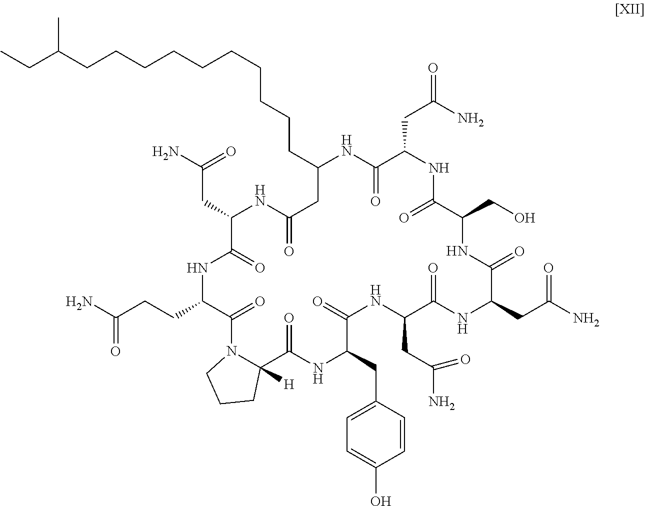 [SMILES: CCC(C)CCCCCCCCCCC1CC(=O)N[C@@H](CC(N)=O)C(=O)N[C@@H](CCC(N)=O)C(=O)N2CCC[C@H]2C(=O)N[C@H](Cc2ccc(O)cc2)C(=O)N[C@H](CC(N)=O)C(=O)N[C@H](CC(N)=O)C(=O)N[C@H](CO)C(=O)N[C@@H](CC(N)=O)C(=O)N1]

The Mojavensin A, or active derivative thereof, may be the C₁₆ isoform. In one embodiment, Mojavensin A may have a structure as set out in formula XIII:

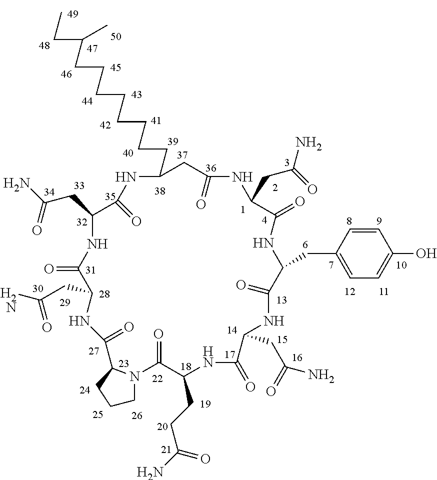 [SMILES: CCC(C)CCCCCCCCC1CC(=O)N[C@H](CC(N)=O)C(=O)N[C@H](Cc2ccc(O)cc2)C(=O)N[C@@H](CC(N)=O)C(=O)N[C@@H](CCC(N)=O)C(=O)N2CCC[C@H]2C(=O)N[C@@H](CC(N)=O)C(=O)N[C@@H](CC(N)=O)C(=O)N1]

The Kurstakin, or active derivative thereof, may be the C₁₃ isoform. Preferably, the Kurstakin is the C₁₅ Kurstakin isoform. In one embodiment, Kurstakin may have a structure as set out in formula XIV:

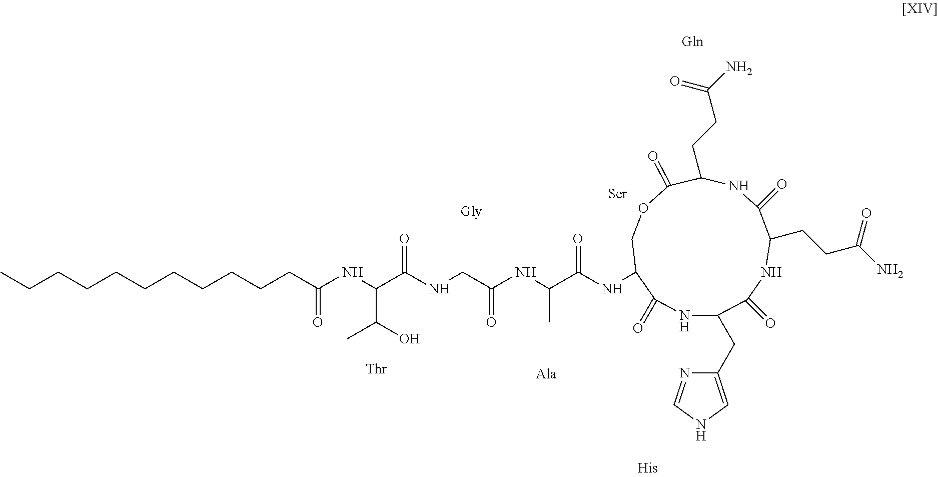 [SMILES: CCCCCCCCCCCC(=O)NC(C(=O)NCC(=O)NC(C)C(=O)NC1COC(=O)C(CCC(N)=O)NC(=O)C(CCC(N)=O)NC(=O)C(Cc2c[nH]cn2)NC1=O)C(C)O]

Preferably, the lipopeptide is Fengycin A, and, in a preferred embodiment, the antibiotic composition comprises the lipopeptides Iturin A, Surfactin and Fengycin A.

Preferably, the live or dead bacterial spore, live or dead vegetative bacterium, the extracellular material produced by the live cell or the disrupted bacterial cell homogenate further produces or comprises the lipopeptides Iturin A and Surfactin, and at least two further lipopeptides selected from a group consisting of: Fengycin A; Fengycin B; Mycosubtilin; Mojavensin A; and Kurstakin, or an active derivative of any of these lipopeptides.

Preferably, the live or dead bacterial spore, live or dead vegetative bacterium, the extracellular material produced by the live cell or the disrupted bacterial cell homogenate further produces or comprises the lipopeptides Iturin A and Surfactin, and at least three further lipopeptides selected from a group consisting of: Fengycin A; Fengycin B; Mycosubtilin; Mojavensin A; and Kurstakin, or an active derivative of any of these lipopeptides.

Preferably, the live or dead bacterial spore, live or dead vegetative bacterium, the extracellular material produced by the live cell or the disrupted bacterial cell homogenate further produces or comprises the lipopeptides Iturin A and Surfactin, and at least four further lipopeptides selected from a group consisting of: Fengycin A; Fengycin B; Mycosubtilin; Mojavensin A; and Kurstakin, or an active derivative of any of these lipopeptides.

In a most preferred embodiment, however, the live or dead bacterial spore, live or dead vegetative bacterium, the extracellular material produced by the live cell or the disrupted bacterial cell homogenate further produces or comprises a the lipopeptides Iturin A, Surfactin, Fengycin A, Fengycin B, Mycosubtilin, Mojavensin A and Kurstakin, or active derivative thereof.

In another preferred embodiment, the bacterium of the invention may comprise a Malonyl CoA-acyl carrier protein transacylase gene from B. amyloliquefaciens NCIMB 42971, which is provided herein as SEQ ID No: 7, as follows:

[SEQ ID No: 7]
ATGAACAATCTTGCCTTTTTATTTCCTGGACAAGGGTCTCAATTTGTAG
GAATGGGCAAACAATTTTGGAATGATTTTGTGCTCGCAAAGAGATTGTT
TGAAGAAGCGAGCGATGCGATCTCCTTGGATGTAAAAAAACTGTGTTTT
AACGGAGATATGAATGAATTGACAAAGACAATGAACGCGCAGCCCGCTA
TTTTAACGGTCAGTGTTATTGCTTTTCAAGTGTATATGCAGGAAATAGG
GGTGAAGCCCCGCTTCCTGGCAGGCCATAGCTTAGGCGAATATTCAGCG
CTTGTCTGTGCCGGCGCCCTTTCTTTTCAGGATGCCGTTACACTTGTAA
GGCAGCGGGGAATTCTTATGCAGAATGCGGATCCTCAGCAGCAGGGGAC
GATGGCCGCCGTGACTCACCTCTCTCTTCAAACGTTGCAGGAAATATGT
TCGAAAGTGTCGACGGAAGACTTTCCGGCAGGAGTAGCCTGCATGAATT
CAGAACAGCAGCATGTGATTTCCGGACACCGGCAAGCTGTGGAACGTGT
CATCAAGATGGCGGAGGAAAAGGGAGCGGCATACACTTATTTGAATGTC
AGTGCGCCTTTTCACAGTTCGCTGATACGATCAGCATCTGAACAATTCC
AGACTGTATTACACCGGTATTCCTTCCGGGATGCCGCATGGCCGATCAT
TTCAAATGTCACCGCACGCCCTTACAGCAGCGGAAATTCAATCAGCGAA
CATCTCGAGCAGCACATGACGATGCCGGTAAGATGGACGGAATCGATGC
ATTACTTGCTTTTACACGGAGTCACAGAAGTCATCGAAATGGGTCCGAA
CAATGTCTTAGCCGGTCTGCTGAGAAAAACAACGAATCACATTGTACCT
TATCCCTTAGGACAGACATCTGATGTTCACTTGCTTTCCAATTCAGCAG
AAAGAAAGAAACATATTGTCCGTTTACGCAAAAAACAACTGAATAAATT
GATGATTCAATCCGTCATTGCGCGAAATTACAACAAGGATTCAGCGGCT
TATTCCAATATGACGACGGCATTATTTACGCAAATCCAAGAGCTGAAAG
AGAGAATGGAAAGACATGAAAATGAGCTCTCAGAACAAGAGCTCGAACA
TTCGATCCATTTATGCAAATTAATTTGCGAGGCTAAACAGCTTCCGGAT
TGGGAAGAATTGCGGATTTTAAAATAA

Accordingly, preferably the bacterium comprises a nucleotide sequence substantially as set out in SEQ ID No: 7, or a variant or fragment thereof.

In one embodiment, the Malonyl CoA-acyl carrier protein transacylase gene from B. amyloliquefaciens NCIMB 42971 may encode an amino acid sequence provided herein as SEQ ID No:9, as follows:

[SEQ ID No: 9]
MNNLAFLFPGQGSQFVGMGKQFWNDFVLAKRLFEEASDAISLDVKKLCF
NGDMNELTKTMNAQPAILTVSVIAFQVYMQEIGVKPRFLAGHSLGEYSA
LVCAGALSFQDAVTLVRQRGILMQNADPQQQGTMAAVTHLSLQTLQEIC
SKVSTEDFPAGVACMNSEQQHVISGHRQAVERVIKMAEEKGAAYTYLNV
SAPFHSSLIRSASEQFQTVLHRYSFRDAAWPIISNVTARPYSSGNSISE
HLEQHMTMPVRWTESMHYLLLHGVTEVIEMGPNNVLAGLLRKTTNHIVP
YPLGQTSDVHLLSNSAERKKHIVRLRKKQLNKLMIQSVIARNYNKDSAA
YSNMTTALFTQIQELKERMERHENELSEQELEHSIHLCKLIC EAKQLP
DWEELRILK

Accordingly, preferably the bacterium comprises a gene encoding the amino acid sequence substantially as set out in SEQ ID No: 9, or a variant or fragment thereof.

In another preferred embodiment, the bacterium of the invention may comprise a Malonyl CoA-acyl carrier protein transacylase gene from B. amyloliquefaciens NCIMB 42971, which is provided herein as SEQ ID No: 8, as follows:

[SEQ ID No: 8]
ATGTATACCAGTCAATTCCAAACCTTAGTAGATGTCATTCGGGAAAGAA
GCAATATCTCTGACCGCGGGATCCGTTTTATCGAATCCGATAAAAACGA
GACGGTTGTCTCTTATCGCCAATTGTTTGAAGAGGCGCAAGGGTATCTT
GGCTATTTACAGCATATCGGCATTCAGCCGAAGCAGGAAATTGTATTTC
AAATCCAAGAAAACAAATCATTTGTCGTTGCTTTTTGGGCTTGTATATT
AGGAGGAATGATCCCGGTGCCGGTCAGTATCGGAGAAGATGATGACCAT
AAGCTGAAGGTCTGGCGCATTTGGAATATATTAAATCACCCGTTTCTGA
TTGCCTCTGAAAAAGTATTGGACAAAATAAAGAAATACGCTGCAGAACA
CGATTTACAGGATTTCCATCATCAATTAAACGAAAAATCTGACATCATT
CAAGATCAAACCTACGATTACCCCGCTTCGTTTTATGAACCTGATGCGG
ATGAACTCGCCTTTATCCAATTTTCTTCAGGATCGACAGGAGATCCAAA
AGGAGTCATGTTAACGCATCACAACTTAATACATAACACGTGCGCCATT
GGGAATGCCCTAGCCGTTCATTCGAGAGACTCTTTCTTATCATGGATGC
CTTTAACGCATGATATGGGGCTCATCGCCTGCCACCTTGTTCCCTTCAT
AACCGGAATCAATCAAAATCTGATGCCTACAGAATTATTTATTCGCAGA
CCTATTCTTTGGATGAAAAAAGCTCATGAACATAAAGCCAGTATTCTAT
CCTCTCCTAATTTCGGATACAACTACTTCCTTAAATTTCTGAAAAACGA
ACCAGACTGGGATTTATCCCACATCAAGGTCATCGCAAACGGTGCAGAA
CCGATATTGCCGGAGCTCTGTGACGAATTTTTGAAAAGATGCGCAGCAT
TCAATCTGAAAAGATCCGCCATTTTGAATGTTTACGGTTTAGCGGAAGC
TTCGGTCGGCGCAGCATTCTCTAAATTAGGTAAAGAATTCGTTCCCGTT
TATTTGCATCGCGATCATTTAAATCTCGGTGAAAGAGCTGTAAACGTCA
GCAAAGAGGATCAAAATTGCGCTTCATTCGTCGAAGTGGGACGACCTAT
TGACTATTGTCAGCTTCGGATCTCCGATGAAGCAAATGAAAGAGTAGAA
GACGGAATCATCGGCCATATCCAGATCAAAGGAGACAATGTGACTCAAG
GGTATTATAACAACCCCGAGAGTACGGAAAAAGCGCTGACTTCTGACGG
CTGGGTAAAAACGGGAGACCTCGGATTCATTAGTGAAAGTGGTAACTTA
GTCGTAACCGGAAGAGAAAAGGACATTATTTTCGTGAACGGAAAAAATA
TCTACCCGCACGATATTGAACGGGTAGCGATTGAAATGGAAGAGGTTGA
CTTAGGAAGGGTTGCCGCCTGCGGTGTATATGATCAAAAGACACAAAGC
GGAGAAATCGTGCTCTTTGTTGTTTACAAAAAATCACCTGAAAAATTCG
CACCGCTTGTCAAAGAGATAAAAAAGCATTTGCTCAAGCGGGGCGGCTG
GAGCATAAAAGATGTCCTTCCGATCCGAAAACTCCCTAAAACAACCAGC
GGAAAGGTTAAACGCTACGAACTTGCCAGACAGTATGAGGCAGGGAATT
TTTCAACAGAGTCTGCCGCCATCAATGAATATTTGGAGAGCAGCCCGGA
AACGTCCGGACAGACTCCCATTCATGAAATTGAAACGGAATTACTGTCT
ATCTTTTCCGATGTGCTCAATGGGAAAAAGGTTCACCTCGCTGACAGTT
ATTTTGATATGGGAGCAAATTCATTACAGTTATCGCAGATTGCCGAGCG
CATAGAACAGAAATTCGGACGCGAGCTTGCCGTTTCAGATCTCTTTACG
TATCCTTCTATCACTGATTTAGCGGCGTATCTGTCTGAAAGCCGGGCTG
AAATCAAGCAGGACGTGGCAGCTAAACCAAGCCATGTGACACCGAAAGA
TATCGCCATTATCGGGATGTCGCTCAATGTCCCTGGAGCATCAACTAAA
AATGATTTTTGGAATCTGCTTGAAAAAGGTGAGCACAGCATTCGAGAAT
ACCCTGCATCCCGGCTGAAAGATGCGGCGGATTATTTAAAGTCCATCCA
AAGCGAAATCAATGAGAATCAGTTTGTGAAGGGCGGCTATTTAGATGAA
ATCGACCGCTTTGATTTCTCGTTCTTCGGTTTAGCTCCTAAAACGGCTC
AGTTTATGGACCCTAACCAAAGACTGTTTTTGCAGTCTGCATGGCATGC
GATTGAAGATGCGGGCTATGCCGGCGGCAGCATGAACGGGAGCCGTGTC
GGGGTATATGCAGGGTACTCGAAGGTGGGCTACGATTATGAACGTCTCC
TTTCTGCGAATTATCCGGAGGAGCTTCATCAATATATCGTGGGCAATCT
CCCTTCCGTGTTAGCCAGCCGAATCGCTTATTTCTTAAATTTAAAAGGG
CCGGCGGTCACAGTCGATACGGCGTGCTCCTCATCGCTTGCCGCCGTTC
ATATGGCATGTAAATCTTTAATATCCGGCGATTGTGAAATGGCTCTTGC
CGGCGGTATCCGGACATCGCTCTTGCCGATCTGTATCGGACTTGATATG
GAATCTTCGGACGGGTACACGAAAACGTTCAGCAAAGATTCAGACGGTA
CTGGCACAGGTGAAGGCGCGGCCGCAGTCCTGCTGAAACCTCTGCAGGA
TGCTGTTCGCGACGGAGACCATATTTACGGCGTAATCAAGGGAAGCGCG
TTGAATCAAGACGGAACAACCGCCGGGATTACAGCACCGAATCCGGCAG
CTCAGACTGAGGTCATTGAGACGGCCTGGAAAGACGCGGGCATTGCCCC
TGAAACACTGTCTTTCATCGAAGCGCATGGCACCGGAACGAAGCTCGGC
GATCCGGTTGAATTTAACGGGCTTTGTAAAGCGTTTGAAAAGTATACGG
CAAAAAAACAATTTTGTGCGATTGGTTCTGTTAAATCGAACATCGGTCA
TTTGTTTGAAGCGGCAGGCATCGTCGGGCTGATCAAATCTGTCCTCATG
CTGAATCACAAGAAAAATCCGCCGTTAGTGCACTTTAATGAACCTAATC
CGCTCATTCATTTTCACTCTTCACCATTTTACGTAAACCAGGAAGCTGC
AGCGTTCCCATCCGGTGATGAGCCGCTGCGAGGCGGAGTCAGCTCATTT
GGCTTTAGCGGAACGAACGCTCATGTGGTATTGGAAGAATATATTTCTC
AAAGTGAGTATGCGCCCGAGGATGAACATGGGCCGCACCTATTTGTTTT
ATCCGCTCATACTGAAAAATCACTCTATGAACTCGCACAGCAGTACCGG
CAATATGTATCGGATGACAGCCAAGCTTCATTAAAGTCCATTTGCTATA
CAGCCAGTACGGGCAGGGCTCATTTGGATCATGGCATTGCCATGATTGT
ATCCGGTAAACAAGAACTATCGGATAAGCTGACCCGCCTGATTCAGGGA
GACAGAAACCTTCCCGGTGTATACATCGGCTACAAGAATATGAAGGAAA
TGCTGCCCGCTCATAAAGAAGAGCTGAATAAACAAGCAGCCGCACTGAT
TAAGCAGCGTTTACGTACGCAAGATGAACGGATCACATGGCTGCATCGC
GCCGCCGAATTATTTGTGCAAGGAGCCGTTATCGATTGGCGCGCGCTTT
ATTCAGGTGAAACTGTACAAAAGACGCCATTGCCCTTGTATCCGTTTGA
ACGGAGCCGATGCTGGGCTGAAGCTGACCAATTGCGCTTAAACGAGGAC
GAAAAGAGAGGAGAAGCGGCATTGAATATCAATCAATCGAAGTCGCATA
TTGAATCCTTCCTGAAAACTGTAATCAGCAATACTTCGGGGATCAGAGC
GGAGGAACTCGATCTGAATGCTCATTTTATCGGACTCGGAATGGATTCT
ATCATGCTGTCACAGGTCAAAAAAGCCATCGCGGACGAATTTGGGGCAG
ACATCCCGATGGATCGTTTTTTTGATACGATGAACAACCTTCAAAGTGT
CATAGATTACTTGGCTGAGACCGTTCCAACGTCCTTTGCATCCGCTCCG
CCTCAAGAGAATGTTCCGGCGCAGGAAATGCAGGTCATTTCAGAAGCAC
AGTCTGAATCGGATCGCAGAGAAGGTCATCAAGAGCATATGCTCGAAAA
AATAATCGCTTCTCAGAATCAATTAATTCAGGATACCTTGCAAGCTCAA
TTAAATAGCTTTAATTTGTTGAGAAACAGCGGACATCATTCCGATGAGA
AAGAATACGCTAAAGCGCAAGAGAGATCAATTCCTTCTGTCCAGCAGGG
GCCTCCGGCCGTCACTGCAGAAAAGAAAGCGGCTCAAGAAGCGAAACCC
TATGTTCCTTTCCAGCCTCAGAACCTGCATGAACAGGGACACTATACCG
CACGGCAAAAACAATACTTAGAAGATTTCATCAAGAAATACGCAGATAA
AACGAAAGGTTCCAAACAATATACGGACAACACCCGATTTGCTCATGCA
AACAACCGCAACTTGTCCAGCTTCCGTTCATATTGGAAGGAAATCGTAT
ACCCGATTATCGCCGAACGTTCTGACGGTTCTAAAATGTGGGATATTGA
CGGAAATGAATATATCGATGTCACCATGGGATTCGGGGTTAACCTTTTC
GGGCATCATCCTTCCTTTATTACACAGGTTATCGATGATTCAGCCCGTT
CTTCATTGCCTCCGCTCGGACCGATGTCAGATGTCGCCGGTGAAGTTGC
CGACCGGATCCGCACATGTACCGGGGTAGAAAGGGTCGCTTTCTATAAT
TCCGGAACAGAGGCCGTCATGGTTGCCCTGCGTTTGGCGCGGGCGGCAA
CAGGAAGAAAGAAAGTGGTGGCGTTCTCGGGCTCTTATCACGGCACGTT
TGACGGCGTATTAGGGGTTGCCGGCACAAAAGGCGGAGCTGCGTCTGCG
AATCCGCTGGCTCCTGGTATACTGCAGAGCTTTATGGATGATTTGATTA
TTTTACATTACAACAATCCCGATTCTCTGGACGTGATCCGCAGTCTTGG
TGATGAATTGGCAGCCGTACTGGTGGAACCGGTACAAAGCCGCAGACCG
GATTTGCAGCCGCGGGCATTTTTGAAAGAATTGCGGGCGATCACGCAGC
AATCCGGAACAGCTCTGATTATGGATGAAATTATTACCGGATTTCGGAT
CGGTCTCGGCGGCGCACAGGAATGGTTCGGCATTCAGGCTGATTTAGTG
ACCTACGGAAAAATCATCGGCGGCGGACAGCCGTTAGGGGTAGTTGCCG
GAAAAGCTGAGTTCATGAATGCGATCGACGGGGGTACCTGGCAGTATGG
GGACGATTCCTACCCGCAAGACGAGGCGAAACGCACGTTTGTGGCCGGA
ACCTTCAATACTCATCCGCTTACCATGAGAATGTCATTAGCCGTGCTTC
GTCATTTACAAACCGAGGGAGAACATCTGTATGAGCAGCTTAATCAAAA
AACAGCCTACTTGGTGGATGAGCTGAATCGCTGCTTCGAACAAGCGCAA
GTGCCTATCCGCATGGTTCGATTCGGTTCTTTATTCCGGTTTGTCTCAT
CGCTTGATAATGACTTGTTCTTTTACCATCTCAACTATAAAGGTGTCTA
TGTGTGGGAAGGACGCAACTGCTTCTTGTCTGCGGCGCATACCGCTGAT
GATATCGAAAAGATTATTCAAGCGGTGAAAGACACGGTGGAGGATCTTC
GCCGAGGCGGATTTATTCCGGAAGGCCCGGACTCCCCTGATGGCGGAGG
CCGTAAAAAGTCCGGGACGCGCGAGCTTTCACCTGAACAAAAGCAGTTG
GTTATGGCATCCCATTACGGGAATGAAGCGTCCGCCGCTTTAAACCAGT
CCATTATGCTGAAAGTGGAGGGCGAACTGCAGCATACACCATTAAAACA
AGCCGTCCGGCATATCGTTGGCCGTCATGAAGCTTTACGTACGGTGATT
CATCCCGATGACGAGGTACAGCAAGTGCAGGAACGGATGAATATAGAAA
TACCAGTCATTGATTTTACCGTTCACCCGCATGAACATCGGGAGTCGGA
AATTCAAAAATGGCTGACAGAAGATGCCAAGCGGCCGTTCCATTTCCAT
GAACAAAAGCCTTTGTTTAGAATCCATGTGCTTACATCGGCTCACAATG
AACATCTGATTGTGCTCACGTTCCATCATATCATTGCCGATGGATGGTC
AATCGCCGTATTTGTTCAAGAACTGGAGAGCAACTACGCGGCAATCGTA
CAAGGAAAACCGATTTCACCGAAAGAGGCAGATGTTTCGTTTCGCCAAT
ACTTAGACTGGCAGCAGGCACAGATTGACAGCGGCCATTATGAAGAAGG
GGTCCGTTATTGGCGGCGTCATTTCTCTGAACCGATTCAGCAGCCAATT
CTGCCGAGCACAGGTTCTGTCCGTTATCCGAACGGGTATGAGGGAGACC
GGTGTACCGTCAGGCTTGGACGGCCATTGAGCGAGGCTTTAAGGTCATT
AAGCATTCAGATGAAAAATAGCGTATTTGTGACAATGCTGGGTGCATTT
CATCTTTTTCTGCACCGGCTTACCAAACAGTCAGGCCTTGTGATCGGGA
TCCCCGCAGCAGGTCAATCGCATATAAAACAGCATGATCTGATTGGAAA
TTGCGTCAATATGATTCCGGTGAAGAACACGTCTACTTCAGAAAGCACT
TTAACCGGTTATCTTGGCAGTATGAAAGAAAGCGTGAATCTTGCAATGC
GGCACCAAGCCGTCCCGATGACACTGGTGGCCAGAGAGCTTCCGCACGA
TCAAGTGCCGGATATGCGTATTATCTTTAATTTAGACAGGCCTTTTCGA
AAGCTGCATTTCGGAAAGGCGGAAGCGGAGCCCGTTGCATACCCGGTAA
AATGCACCCTGTACGATTTATTTCTTAACATAACAGACGCGCATCAAGA
ATATGTTCTTGATTTCGACTTTAATACGAACGTCATCAGTCCGGAAATC
ATGAAAAAGTGGGGAGCGGGTTTTACAAATTTGCTGCAAAAAATGGTTG
AGGGGGATTCAATCCCTCTTGACGCCATGATGATGTTTTCCGATGAAGA
ACAGCATGATTTACAAAAACTGTATGCCGAACACCAGAAGCGGGTCTCT
TCAATAGGAAGCAATACAGCAAATTTCACTGAAGCCTACGAGGCGCCGA
TAAATGAAACGGAACGGCAGCTGGCGCGGATTTGGGAGGAACTTTTCGG
CCTTGAACGGGTCGGCAGATCAGATCGCTTTCTGGCTCTGGGAGGAAAC
TCGCTCCAGGCGACGCTTATGCTTTCCAAAATTCAGAAGACATTTCATC
AAAAGGTTTCCATCGGACAATTTTTCAATCACCAGACTGTTAAGGAATT
AGCACATTTCATTCAGAACGAAACAAAAGTCGTGCACCTCCCGATGAAA
GCTGCCGAGAAAAAAGCGTATTACCCGACATCGCCGGCGCAGCAAAGAG
TATATTTCCTGCACCAACTGGAACCGGATCAGCTGGCGCAAAATATGTT
CGGCCAAATATCAATAACAGGGAAGTACGATGAGCAAGCCCTGATCTCA
TCTCTTCAACAAGTGATGCAGCGGCACGAAGCGTTTCGCACGTATTTCG
ACATTATAGATGGCGATATCGTTCAGAAACTTGAAAACGAAGTTGATTT
TAACGTTCATGTCCGGACAATGAGCCGGGACGAATTTGATGCCTATTCA
GACCGGTTTGTAAAACCGTTCCGCCTGGACCAAGCTCCGTTAGTTCGTG
CGGAGCTGATCAAGATTGAAAACGAGCAGGCCGAACTGCTCATCGATAT
GCATCATATCATTTCGGATGGTTATTCCGTCAACATCCTTACAAATGAA
TTGCTGGCTTTATATCATCAGAAACCATTACCGGACATTGAATTTGAAT
ATAAAGATTTCGCAGAATGGCAAAACCAACGGCTGAATGAGGATGCCAT
GAAGCGGCAGGAGACATATTGGCTGGAACAATTTCAAGACGAAATTCCC
ATCCTTGACCTGCCGACAGACGGTTCAAAAGCGGCAGAACGGTCTTCTG
AGGGACAGCGTGTGACATGCTCCTTACAGCCGGATGTCATCCGTTCGCT
TCAAGATTTGGCGCAAAAGGCGGAAACCACTCTCTATACGGTGCTTCTG
GCCGCCTATAATGTGCTGCTTCATAAATATACCGGACAAGAAGACATTG
TCGTAGGCACGCCTGCTTCAGGAAGAAATCATCCGGATATCGAAAAAAT
CATCGGTATTTTCATACAAACCATCGGAATCCGGACGAAGCCGCACGCC
AATAGAACGTTTACGGATTATCTGGAAGAAGTAAAGCGGCAGACGCTTG
ACGCTTTCGAAAACCAAGACTATCCATTCGACCGGCTTGTGGAGAAATT
AAATGTGCAGCGGGAAACAACCGGAAAGTCTCTGTTTAACACGATGTTT
GTGTTTCAAAACATTGAATTTCATGAAATCCGGCACAATGAATGTACAT
TTAAAGTGAAAGAACGAAATCCAGGGGTCTCTTTGTATGATTTGATGCT
CACGATCGAAGATGCCGGTCAACAGATAGAGATGCATTTTGATTATAAA
CCGGGACGATTCACAAAAGACACCATTGAACAGATCACCAGACACTATA
CCGGCATTTTAAACAGTCTTGTTGAGCAGCCGGAGATGACATTGTCTTC
CGTTCCTATGCTGTCTGAAACCGAACGGCATCAACTGCTGACGGAGTGT
AACGGCACAAAGACGCCGTATCCGCATAACGAAACAGTAACCCGATGGT
TTGAAATGCAGGCGGAACAGAGTCCCGATCATGCAGCCGTTATTTTTGG
CAATGAGCGGTATACGTACAGACAGCTCAATGAACGGGCGAACCGATTG
GCGCGGACGTTACGGACAAAAGGCGTACAAGCGGATCAATTCGTTGCCA
TCATCTCTCCGCATCGCATCGAGTTGATTGTTGGTATTTTGGCTGTTCT
GAAATCAGGCGGCGCATACGTGCCTATTGATCCTGAATATCCGGAAGAT
CGGATCCAATATATGCTGAGAGATTCAAGGGCGGAGGTTGTGTTGACAC
AGCGCAGCCTGCTGGATCAATTACCGTATGATGGTGACGTTGTGCTTTT
GGATGAGGAAAACTCATACCATGAGGATCACTCGAATCTTGAATCGGAC
AGCGATGCGCATGATTTGGCCTACATGATCTATACGTCAGGTTCCACGG
GAAATCCAAAAGGTGTCCTCATTGAGCATCAGGGACTGGCTGATTATAT
TTGGTGGGCGAAAGAGGTTTATGTAAGAGGTGAGAAAACCAACTTCCCA
TTATACTCTTCCATCTCTTTCGATCTGACTGTGACCTCGATATTTACAC
CGCTGGTTACGGGAAATACCATCATTGTCTTTGACGGCGAAGACAAAAG
CGCTGTGCTTTCTGAGATTATGCGGGACTCAAGAATAGACATGATCAAA
TTGACCCCGGCACATCTGCACGTCATCAAGGAGATGAATATCGGTGGCG
GCACCGCAATACGGAAAATGATTGTCGGCGGAGAAAATTTAAGCACCCG
TCTGGCCAAAAGTGTCAGCGAGCAGTTTAAAGGCCGGCTGGACATTTTC
AATGAATACGGACCGACGGAAGCTGTCGTCGGATGTATGATTTATCACT
TCGACGCAGAACGGGACAAGCGGGAATTTGTACCGATCGGCACTCCGGC
TGCCAACACGGATATTTATGTGGCCGATGCAAGCAGAAATCTGGTTCCG
ATCGGGGTAATCGGCGAAATATATATCAGCGGACCGGGTGTTGCCAGAG
GGTATTGGAACCGTCCGGATTTAACGGCAGAGAAATTTGTTGAAAACCC
GTATGTCCCGGGAGCGAAGATGTACAAATCAGGGGATTTGGCTAAGCGG
TTGAAGGACGGAAACCTTGTATATATCGGGCGCGTTGATGAACAAGTCA
AAATCAGGGGATACCGAATCGAGCTTGGTGAAATTGAAGCAGCAATGCA
TAACGCGGAAGCGGTGCAAAAAGCCGCGGTTACAGTGAAAGAAGAAGAA
GACGGCTTAAAACAATTATGCGCGTATTACGTAAGCGACAAGCCTATAG
CGGCTGCGCAGCTTAGGGAACAATTGTCATCGGAGCTTCCGGACTACAT
GGTTCCGTCCTATTTTGTCCAACTGGAGCATATGCCGTTAACGTCCAAC
GGGAAAATAAACCGTAAGGCACTGCCAGCACCAGAAGCGAGTCTGCAGC
AGACAGCTGAATATGTTCCGCCGGGTAATGAGACGGAGTCCAAACTGAC
AGATTTATGGAAGGAAGTGCTCGGAATAAGCCATGCGGGGATCAAACAT
AATTTCTTTGATCTCGGAGGCAACTCCATCCGAGCGGCTGCCTTGGCCG
CCAGAATTCACAAAGAGCTGGATGTGAATCTGTCTCTCAAAGACATATT
CAAGTTTCCTACCATTGAACAATTGGCTGACAAGGCGTTACACATGGAC
AAAAACCGATATGTACCGATTCCGGCTGCAAAGGAAATGCCATATTATC
CGGTTTCTTCAGCTCAGAGGCGTATGTATTTGTTAAGTCACACAGAAGG
CGGCGAGCTGACTTACAATATGACGGGTGCCATGAATGTGGAAGGGACG
ATCGATCCCGAACGGTTAAACGCCGCTTTCCGAAAATTAATCGCGCGTC
ATGAAGCGTTGCGGACCAGCTTTGATTTATATGAAGGCGAGCCGGCACA
GCGTATTCATCAGAACGTCGACTTTACGATAGAACGGATTCAAGCAAGC
GAAGAAGAAGCGGAAGACCGTGTGCTTGATTTCATCAAAGCGTTTGACT
TAGCCAAACCGCCGCTGATGCGGGCCGGACTGATTGAAATTGAACCTGC
GCGGCACGTGCTTGTGGTTGATATGCATCATATCATTTCTGACGGCGTG
TCCGTCAATATTCTGATGAAAGATTTAAGCCGAATCTACGAGGGGAACG
AACCGGACCCGCTCTCTATTCAATATAAAGACTTTGCAGTTTGGCAGCA
ATCGGACATTCAGAAACGGAACATCAAGAGCCAGGAAGCGTATTGGCTG
GATCAGTTTCACAGTGATATTCCTGTACTGGATATGCCTGCGGATTATG
AGAGACCTGCCATACGCGATTACGAAGGCGAATCATTTGAATTTCTTAT
ACCCGAACACTTGAAACAGCGTTTAAGCCAAATGGAAGAAGACACAGGA
GCAACACTGTATATGATTTTATTGGCGGCCTATACGATTCTTTTATCCA
GGTACAGCGGACAAGAAGATATTATCGTAGGAACGCCATCTGCCGGGCG
GACTCATTTGGATGTAGAGCCGGTCGTGGGAATGTTCGTCAATACGTTA
GTCATTCGCAATCACCCGGCGGGCCGTAAAACATTTGATGCCTACTTAA
ACGAAGTAAAAGAAAACATGCTGAACGCCTATAAAAATCAAGACTATCC
ATTGGAAGAATTAATTCAGCATCTGCATCTTCCAAAAGATTCAAGCCGC
AATCCTTTATTCGATACGATGTTTGTGCTGCAAAATCTCGATCATGCTG
AATTGACTTTCGATTCTCTTCAACTCAAGCCGTATTCATTTCATCATCC
GGTTGCCAAATTCGATTTGACCTTGTCGATTCAGGCGGACCAAGACAAC
TATCACGGACTGTTTGAATATTCGAAAAAACTGTTTAAGAAAAGCAGAA
TCGAGGTTTTATCAAACGACTACTTACACATTCTATCGGCGATTTTGGA
ACAACCAAGCATTCTAATTGAACATATCGGATTGAGCGGCAGCAATGAG
GAAGAAGAGAACGCGCTTGATTCTATTCAATTGAACTTTTAG

Accordingly, preferably the bacterium comprises the nucleotide sequence substantially as set out in SEQ ID No: 8, or a variant or fragment thereof.

In one embodiment, Malonyl CoA-acyl carrier protein transacylase gene from B. amyloliquefaciens NCIMB 42971 may encode an amino acid sequence provided herein as SEQ ID No:10, as follows:

[SEQ ID No: 10]
MYTSQFQTLVDVIRERSNISDRGIRFIESDKNETVVSYRQLFEEAQGYL
GYLQHIGIQPKQEIVFQIQENKSFVVAFWACILGGMIPVPVSIGEDDDH
KLKVWRIWNILNHPFLIASEKVLDKIKKYAAEHDLQDFHHQLNEKSDII
QDQTYDYPASFYEPDADELAFIQFSSGSTGDPKGVMLTHHNLIHNTCAI
GNALAVHSRDSFLSWMPLTHDMGLIACHLVPFITGINQNLMPTELFIRR
PILWMKKAHEHKASILSSPNFGYNYFLKFLKNEPDWDLSHIKVIANGAE
PILPELCDEFLKRCAAFNLKRSAILNVYGLAEASVGAAFSKLGKEFVPV
YLHRDHLNLGERAVNVSKEDQNCASFVEVGRPIDYCQLRISDEANERVE
DGIIGHIQIKGDNVTQGYYNNPESTEKALTSDGWVKTGDLGFISESGNL
VVTGREKDIIFVNGKNIYPHDIERVAIEMEEVDLGRVAACGVYDQKTQS
GEIVLFVVYKKSPEKFAPLVKEIKKHLLKRGGWSIKDVLPIRKLPKTTS
GKVKRYELARQYEAGNFSTESAAINEYLESSPETSGQTPIHEIETELLS
IFSDVLNGKKVHLADSYFDMGANSLQLSQIAERIEQKFGRELAVSDLFT
YPSITDLAAYLSESRAEIKQDVAAKPSHVTPKDIAIIGMSLNVPGASTK
NDFWNLLEKGEHSIREYPASRLKDAADYLKSIQSEINENQFVKGGYLDE
IDRFDFSFFGLAPKTAQFMDPNQRLFLQSAWHAIEDAGYAGGSMNGSRV
GVYAGYSKVGYDYERLLSANYPEELHQYIVGNLPSVLASRIAYFLNLKG
PAVTVDTACSSSLAAVHMACKSLISGDCEMALAGGIRTSLLPICIGLDM
ESSDGYTKTFSKDSDGTGTGEGAAAVLLKPLQDAVRDGDHIYGVIKGSA
LNQDGTTAGITAPNPAAQTEVIETAWKDAGIAPETLSFIEAHGTGTKLG
DPVEFNGLCKAFEKYTAKKQFCAIGSVKSNIGHLFEAAGIVGLIKSVLM
LNHKKNPPLVHFNEPNPLIHFHSSPFYVNQEAAAFPSGDEPLRGGVSSF
GFSGTNAHVVLEEYISQSEYAPEDEHGPHLFVLSAHTEKSLYELAQQYR
QYVSDDSQASLKSICYTASTGRAHLDHGIAMIVSGKQELSDKLTRLIQG
DRNLPGVYIGYKNMKEMLPAHKEELNKQAAALIKQRLRTQDERITWLHR
AAELFVQGAVIDWRALYSGETVQKTPLPLYPFERSRCWAEADQLRLNED
EKRGEAALNINQSKSHIESFLKTVISNTSGIRAEELDLNAHFIGLGMDS
IMLSQVKKAIADEFGADIPMDRFFDTMNNLQSVIDYLAETVPTSFASAP
PQENVPAQEMQVISEAQSESDRREGHQEHMLEKIIASQNQLIQDTLQAQ
LNSFNLLRNSGHHSDEKEYAKAQERSIPSVQQGPPAVTAEKKAAQEAKP
YVPFQPQNLHEQGHYTARQKQYLEDFIKKYADKTKGSKQYTDNTRFAHA
NNRNLSSFRSYWKEIVYPIIAERSDGSKMWDIDGNEYIDVTMGFGVNLF
GHHPSFITQVIDDSARSSLPPLGPMSDVAGEVADRIRTCTGVERVAFYN
SGTEAVMVALRLARAATGRKKVVAFSGSYHGTFDGVLGVAGTKGGAASA
NPLAPGILQSFMDDLIILHYNNPDSLDVIRSLGDELAAVLVEPVQSRRP
DLQPRAFLKELRAITQQSGTALIMDEIITGFRIGLGGAQEWFGIQADLV
TYGKIIGGGQPLGVVAGKAEFMNAIDGGTWQYGDDSYPQDEAKRTFVAG
TFNTHPLTMRMSLAVLRHLQTEGEHLYEQLNQKTAYLVDELNRCFEQAQ
VPIRMVRFGSLFRFVSSLDNDLFFYHLNYKGVYVWEGRNCFLSAAHTAD
DIEKIIQAVKDTVEDLRRGGFIPEGPDSPDGGGRKKSGTRELSPEQKQL
VMASHYGNEASAALNQSIMLKVEGELQHTPLKQAVRHIVGRHEALRTVI
HPDDEVQQVQERMNIEIPVIDFTVHPHEHRESEIQKWLTEDAKRPFHFH
EQKPLFRIHVLTSAHNEHLIVLTFHHIIADGWSIAVFVQELESNYAAIV
QGKPISPKEADVSFRQYLDWQQAQIDSGHYEEGVRYWRRHFSEPIQQPI
LPSTGSVRYPNGYEGDRCTVRLGRPLSEALRSLSIQMKNSVFVTMLGAF
HLFLHRLTKQSGLVIGIPAAGQSHIKQHDLIGNCVNMIPVKNTSTSEST
LTGYLGSMKESVNLAMRHQAVPMTLVARELPHDQVPDMRIIFNLDRPFR
KLHFGKAEAEPVAYPVKCTLYDLFLNITDAHQEYVLDFDFNTNVISPEI
MKKWGAGFTNLLQKMVEGDSIPLDAMMMFSDEEQHDLQKLYAEHQKRVS
SIGSNTANFTEAYEAPINETERQLARIWEELFGLERVGRSDRFLALGGN
SLQATLMLSKIQKTFHQKVSIGQFFNHQTVKELAHFIQNETKVVHLPMK
AAEKKAYYPTSPAQQRVYFLHQLEPDQLAQNMFGQISITGKYDEQALIS
SLQQVMQRHEAFRTYFDIIDGDIVQKLENEVDFNVHVRTMSRDEFDAYS
DRFVKPFRLDQAPLVRAELIKIENEQAELLIDMHHIISDGYSVNILTNE
LLALYHQKPLPDIEFEYKDFAEWQNQRLNEDAMKRQETYWLEQFQDEIP
ILDLPTDGSKAAERSSEGQRVTCSLQPDVIRSLQDLAQKAETTLYTVLL
AAYNVLLHKYTGQEDIVVGTPASGRNHPDIEKIIGIFIQTIGIRTKPHA
NRTFTDYLEEVKRQTLDAFENQDYPFDRLVEKLNVQRETTGKSLFNTMF
VFQNIEFHEIRHNECTFKVKERNPGVSLYDLMLTIEDAGQQIEMHFDYK
PGRFTKDTIEQITRHYTGILNSLVEQPEMTLSSVPMLSETERHQLLTEC
NGTKTPYPHNETVTRWFEMQAEQSPDHAAVIFGNERYTYRQLNERANRL
ARTLRTKGVQADQFVAIISPHRIELIVGILAVLKSGGAYVPIDPEYPED
RIQYMLRDSRAEVVLTQRSLLDQLPYDGDVVLLDEENSYHEDHSNLESD
SDAHDLAYMIYTSGSTGNPKGVLIEHQGLADYIWWAKEVYVRGEKTNFP
LYSSISFDLTVTSIFTPLVTGNTIIVFDGEDKSAVLSEIMRDSRIDMIK
LTPAHLHVIKEMNIGGGTAIRKMIVGGENLSTRLAKSVSEQFKGRLDIF
NEYGPTEAVVGCMIYHFDAERDKREFVPIGTPAANTDIYVADASRNLVP
IGVIGEIYISGPGVARGYWNRPDLTAEKFVENPYVPGAKMYKSGDLAKR
LKDGNLVYIGRVDEQVKIRGYRIELGEIEAAMHNAEAVQKAAVTVKEEE
DGLKQLCAYYVSDKPIAAAQLREQLSSELPDYMVPSYFVQLEHMPLTSN
GKINRKALPAPEASLQQTAEYVPPGNETESKLTDLWKEVLGISHAGIKH
NFFDLGGNSIRAAALAARIHKELDVNLSLKDIFKFPTIEQLADKALHMD
KNRYVPIPAAKEMPYYPVSSAQRRMYLLSHTEGGELTYNMTGAMNVEGT
IDPERLNAAFRKLIARHEALRTSFDLYEGEPAQRIHQNVDFTIERIQAS
EEEAEDRVLDFIKAFDLAKPPLMRAGLIEIEPARHVLVVDMHHIISDGV
SVNILMKDLSRIYEGNEPDPLSIQYKDFAVWQQSDIQKRNIKSQEAYWL
DQFHSDIPVLDMPADYERPAIRDYEGESFEFLIPEHLKQRLSQMEEDTG
ATLYMILLAAYTILLSRYSGQEDIIVGTPSAGRTHLDVEPVVGMFVNTL
VIRNHPAGRKTFDAYLNEVKENMLNAYKNQDYPLEELIQHLHLPKDSSR
NPLFDTMFVLQNLDHAELTFDSLQLKPYSFHHPVAKFDLTLSIQADQDN
YHGLFEYSKKLFKKSRIEVLSNDYLHILSAILEQPSILIEHIGLSGSNE
EEENALDSIQLNF

Accordingly, preferably the bacterium comprises a gene encoding the amino acid sequence substantially as set out in SEQ ID No: 10, or a variant or fragment thereof.

In another embodiment, the bacterium of the invention may comprise 16S rDNA.

Thus, in another preferred embodiment, the bacterium may comprise 16S rDNA of NCIMB 42971, which is provided herein as SEQ ID No: 1, as follows:

[SEQ ID No: 1]
TTTATCGGAGAGTTTGATCCTGGCTCAGGACGAACGCTGGCGGCGTGCC
TAATACATGCAAGTCGAGCGGACAGATGGGAGCTTGCTCCCTGATGTTA
GCGGCGGACGGGTGAGTAACACGTGGGTAACCTGCCTGTAAGACTGGGA
TAACTCCGGGAAACCGGGGCTAATACCGGATGGTTGTCTGAACCGCATG
GTTCAGACATGAAAGGTGGCTTCGGCTACCACTTACAGATGGACCCGCG
GCGCATTAGCTAGTTGGTGAGGTAACGGCTCACCAAGGCGACGATGCGT
AGCCGACCTGAGAGGGTGATCGGCCACACTGGGACTGAGACACGGCCCA
GACTCCTACGGGAGGCAGCAGTAGGGAATCTTCCGCAATGGACGAAAGT
CTGACGGAGCAACGCCGCGTGAGTGATGAAGGTTTTCGGATCGTAAAGC
TCTGTTGTTAGGGAAGAACAAGTGCCGTTCAAATAGGGCGGCACCTTGA
CGGTACCTAACCAGAAAGCCACGGCTAACTACGTGCCAGCAGCCGCGGT
AATACGTAGGTGGCAAGCGTTGTCCGGAATTATTGGGCGTAAAGGGCTC
GCAGGCGGTTTCTTAAGTCTGATGTGAAAGCCCCCGGCTCAACCGGGGA
GGGTCATTGGAAACTGGGGAACTTGAGTGCAGAAGAGGAGAGTGGAATT
CCACGTGTAGCGGTGAAATGCGTAGAGATGTGGAGGAACACCAGTGGCG
AAGGCGACTCTCTGGTCTGTAACTGACGCTGAGGAGCGAAAGCGTGGGG
AGCGAACAGGATTAGATACCCTGGTAGTCCACGCCGTAAACGATGAGTG
CTAAGTGTTAGGGGGTTTCCGCCCCTTAGTGCTGCAGCTAACGCATTAA
GCACTCCGCCTGGGGAGTACGGTCGCAAGACTGAAACTCAAAGGAATTG
ACGGGGGCCCGCACAAGCGGTGGAGCATGTGGTTTAATTCGAAGCAACG
CGAAGAACCTTACCAGGTCTTGACATCCTCTGACAATCCTAGAGATAGG
ACGTCCCCTTCGGGGGCAGAGTGACAGGTGGTGCATGGTTGTCGTCAGC
TCGTGTCGTGAGATGTTGGGTTAAGTCCCGCAACGAGCGCAACCCTTGA
TCTTAGTTGCCAGCATTCAGTTGGGCACTCTAAGGTGACTGCCGGTGAC
AAACCGGAGGAAGGTGGGGATGACGTCAAATCATCATGCCCCTTATGAC
CTGGGCTACACACGTGCTACAATGGACAGAACAAAGGGCAGCGAAACCG
CGAGGTTAAGCCAATCCCACAAATCTGTTCTCAGTTCGGATCGCAGTCT
GCAACTCGACTGCGTGAAGCTGGAATCGCTAGTAATCGCGGATCAGCAT
GCCGCGGTGAATACGTTCCCGGGCCTTGTACACACCGCCCGTCACACCA
CGAGAGTTTGTAACACCCGAAGTCGGTGAGGTAACCTTTATGGAGCCAG
CCGCCGAAGGTGGGACAGATGATTGGGGTGAAGTCGTAACAAGGTAGCC
GTATCGGAAGGTGCGGCTGGATCACCTCCTTTCTAA

Accordingly, preferably the bacterium comprises the nucleotide sequence substantially as set out in SEQ ID No: 1, or a variant or fragment thereof.

In another preferred embodiment, the bacterium may comprise 16S rDNA of NCIMB 42972, which is provided herein as SEQ ID No: 2, as follows:

[SEQ ID No: 2]
TTTATCGGAGAGTTTGATCCTGGCTCAGGACGAACGCTGGCGGCGTGCC
TAATACATGCAAGTCGAGCGGACAGATGGGAGCTTGCTCCCTGATGTTA
GCGGCGGACGGGTGAGTAACACGTGGGTAACCTGCCTGTAAGACTGGGA
TAACTCCGGGAAACCGGGGCTAATACCGGATGGTTGTCTGAACCGCATG
GTTCAGACATAAAAGGTGGCTTCGGCTACCACTTACAGATGGACCCGCG
GCGCATTAGCTAGTTGGTGAGGTAACGGCTCACCAAGGCGACGATGCGT
AGCCGACCTGAGAGGGTGATCGGCCACACTGGGACTGAGACACGGCCCA
GACTCCTACGGGAGGCAGCAGTAGGGAATCTTCCGCAATGGACGAAAGT
CTGACGGAGCAACGCCGCGTGAGTGATGAAGGTTTTCGGATCGTAAAGC
TCTGTTGTTAGGGAAGAACAAGTGCCGTTCAAATAGGGCGGCACCTTGA
CGGTACCTAACCAGAAAGCCACGGCTAACTACGTGCCAGCAGCCGCGGT
AATACGTAGGTGGCAAGCGTTGTCCGGAATTATTGGGCGTAAAGGGCTC
GCAGGCGGTTTCTTAAGTCTGATGTGAAAGCCCCCGGCTCAACCGGGGA
GGGTCATTGGAAACTGGGGAACTTGAGTGCAGAAGAGGAGAGTGGAATT
CCACGTGTAGCGGTGAAATGCGTAGAGATGTGGAGGAACACCAGTGGCG
AAGGCGACTCTCTGGTCTGTAACTGACGCTGAGGAGCGAAAGCGTGGGG
AGCGAACAGGATTAGATACCCTGGTAGTCCACGCCGTAAACGATGAGTG
CTAAGTGTTAGGGGGTTTCCGCCCCTTAGTGCTGCAGCTAACGCATTAA
GCACTCCGCCTGGGGAGTACGGTCGCAAGACTGAAACTCAAAGGAATTG
ACGGGGGCCCGCACAAGCGGTGGAGCATGTGGTTTAATTCGAAGCAACG
CGAAGAACCTTACCAGGTCTTGACATCCTCTGACAATCCTAGAGATAGG
ACGTCCCCTTCGGGGGCAGAGTGACAGGTGGTGCATGGTTGTCGTCAGC
TCGTGTCGTGAGATGTTGGGTTAAGTCCCGCAACGAGCGCAACCCTTGA
TCTTAGTTGCCAGCATTCAGTTGGGCACTCTAAGGTGACTGCCGGTGAC
AAACCGGAGGAAGGTGGGGATGACGTCAAATCATCATGCCCCTTATGAC
CTGGGCTACACACGTGCTACAATGGACAGAACAAAGGGCAGCGAAACCG
CGAGGTTAAGCCAATCCCACAAATCTGTTCTCAGTTCGGATCGCAGTCT
GCAACTCGACTACGTGAAGCTGGAATCGCTAGTAATCGCGGATCAGCAT
GCCGCGGTGAATACGTTCCCGGGCCTTGTACACACCGCCCGTCACACCA
CGAGAGTTTGTAACACCCGAAGTCGGTGAGGTAACCTTTTAGGAGCCAG
CCGCCGAAGGTGGGACAGATGATTGGGGTGAAGTCGTAACAAGGTAGCC
GTATCGGAAGGTGCGGCTGGATCACCTCCTTTCTAA

Accordingly, preferably the bacterium comprises the nucleotide sequence substantially as set out in SEQ ID No: 2, or a variant or fragment thereof.

Thus, in another preferred embodiment, the bacterium may comprise 16S rDNA of NCIMB 42973, which is provided herein as SEQ ID No: 3, as follows:

[SEQ ID No: 3]
TTTATCGGAGAGTTTGATCCTGGCTCAGGACGAACGCTGGCGGCGTGCC
TAATACATGCAAGTCGAGCGGACAGATGGGAGCTTGCTCCCTGATGTTA
GCGGCGGACGGGTGAGTAACACGTGGGTAACCTGCCTGTAAGACTGGGA
TAACTCCGGGAAACCGGGGCTAATACCGGATGGTTGTCTGAACCGCATG
GTTCAGACATAAAAGGTGGCTTCGGCTACCACTTACAGATGGACCCGCG
GCGCATTAGCTAGTTGGTGAGGTAACGGCTCACCAAGGCGACGATGCGT
AGCCGACCTGAGAGGGTGATCGGCCACACTGGGACTGAGACACGGCCCA
GACTCCTACGGGAGGCAGCAGTAGGGAATCTTCCGCAATGGACGAAAGT
CTGACGGAGCAACGCCGCGTGAGTGATGAAGGTTTTCGGATCGTAAAGC
TCTGTTGTTAGGGAAGAACAAGTGCCGTTCAAATAGGGCGGCACCTTGA
CGGTACCTAACCAGAAAGCCACGGCTAACTACGTGCCAGCAGCCGCGGT
AATACGTAGGTGGCAAGCGTTGTCCGGAATTATTGGGCGTAAAGGGCTC
GCAGGCGGTTTCTTAAGTCTGATGTGAAAGCCCCCGGCTCAACCGGGGA
GGGTCATTGGAAACTGGGGAACTTGAGTGCAGAAGAGGAGAGTGGAATT
CCACGTGTAGCGGTGAAATGCGTAGAGATGTGGAGGAACACCAGTGGCG
AAGGCGACTCTCTGGTCTGTAACTGACGCTGAGGAGCGAAAGCGTGGGG
AGCGAACAGGATTAGATACCCTGGTAGTCCACGCCGTAAACGATGAGTG
CTAAGTGTTAGGGGGTTTCCGCCCCTTAGTGCTGCAGCTAACGCATTAA
GCACTCCGCCTGGGGAGTACGGTCGCAAGACTGAAACTCAAAGGAATTG
ACGGGGGCCCGCACAAGCGGTGGAGCATGTGGTTTAATTCGAAGCAACG
CGAAGAACCTTACCAGGTCTTGACATCCTCTGACAATCCTAGAGATAGG
ACGTCCCCTTCGGGGGCAGAGTGACAGGTGGTGCATGGTTGTCGTCAGC
TCGTGTCGTGAGATGTTGGGTTAAGTCCCGCAACGAGCGCAACCCTTGA
TCTTAGTTGCCAGCATTCAGTTGGGCACTCTAAGGTGACTGCCGGTGAC
AAACCGGAGGAAGGTGGGGATGACGTCAAATCATCATGCCCCTTATGAC
CTGGGCTACACACGTGCTACAATGGACAGAACAAAGGGCAGCGAAACCG
CGAGGTTAAGCCAATCCCACAAATCTGTTCTCAGTTCGGATCGCAGTCT
GCAACTCGACTGCGTGAAGCTGGAATCGCTAGTAATCGCGGATCAGCAT
GCCGCGGTGAATACGTTCCCGGGCCTTGTACACACCGCCCGTCACACCA
CGAGAGTTTGTAACACCCGAAGTCGGTGAGGTAACCTTTATGGAGCCAG
CCGCCGAAGGTGGGACAGATGATTGGGGTGAAGTCGTAACAAGGTAGCC
GTATCGGAAGGTGCGGCTGGATCACCTCCTTTCTAA

Accordingly, preferably the bacterium comprises the nucleotide sequence substantially as set out in SEQ ID No: 3, or a variant or fragment thereof.

In another preferred embodiment, the bacterium may comprise 16S rDNA of NCIMB 42974, which is provided herein as SEQ ID No: 4, as follows:

[SEQ ID No: 4]
ACGATGCGTAGCCGACCTGAGAGGGTGATCGGCCACACTGGGACTGAGA
CACGGCCCAGACTCCTACGGGAGGCAGCAGTAGGGAATCTTCCGCAATG
GACGAAAGTCTGACGGAGCAACGCCGCGTGAGTGATGAAGGTTTTCGGA
TCGTAAAGCTCTGTTGTTAGGGAAGAACAAGTACCGTTCGAATAGGGCG
GTACCTTGACGGTACCTAACCAGAAAGCCACGGCTAACTACGTGCCAGC
AGCCGCGGTAATACGTAGGTGGCAAGCGTTGTCCGGAATTATTGGGCGT
AAAGGGCTCGCAGGCGGTTTCTTAAGTCTGATGTGAAAGCCCCCGGCTC
AACCGGGGAGGGTCATTGGAAACTGGGGAACTTGAGTGCAGAAGAGGAG
AGTGGAATTCCACGTGTAGCGGTGAAATGCGTAGAGATGTGGAGGAACA
CCAGTGGCGAAGGCGACTCTCTGGTCTGTAACTGACGCTGAGGAGCGAA
AGCGTGGGGAGCGAACAGGATTAGATACCCTGGTAGTCCACGCCGTAAA
CGATGAGTGCTAAGTGTTAGGGGGTTTCCGCCCCTTAGTGCTGCAGCTA
ACGCATTAAGCACTCCGCCTGGGGAGTACGGTCGCAAGACTGAAACTCA
AAGGAATTGACGGGGGCCCGCACAAGCGGTGGAGCATGTGGTTTAATTC
GAAGCAACGCGAAGAACCTTACCAGGTCTTGACATCCTCTGACAATCCT
AGAGATAGGACGTCCCCTTCGGGGGCAGAGTGACAGGTGGTGCATGGTT
GTCGTCAGCTCGTGTCGTGAGATGTTGGGTTAAGTCCCGCAACGAGCGC
AACCCTTGATCTTAGTTGCCAGCATTCAGTTGGGCACTCTAAGGTGACT
GCCGGTGACAAACCGGAGGAAGGTGGGGATGACGTCAAATCATCATGCC
CCTTATGACCTGGGCTACACACGTGCTACAATGGACAGAACAAAGGGCA
GCGAAACCGCGAGGTTAAGCCAATCCCACAAATCTGTTCTCAGTTCGGA
TCGCAGTCTGCAACTCGACTGCGTGAAGCTGGAATCGCTAGTAATCGCG
GATCAGCATGCCGCGGTGAATACGTTCCCGGGCCTTGTACACACCGCCC
GTCACACCACGAGAGTTTGTAACACCCGAAGTCGGTGAGGTAACCTTTT
AGGAGCCAGCCGCCGAAGGTGGGACAGATGATTGGGGTGAAGTCGTAAC
AAGGTAGCCGTATCGGAAGGTGCGGCTGGATCACCTCCTTTCTAAGGA

Accordingly, preferably the bacterium comprises the nucleotide sequence substantially as set out in SEQ ID No: 4, or a variant or fragment thereof.

In another preferred embodiment, the bacterium may comprise 16S rDNA of NCIMB 43393, which is provided herein as SEQ ID No: 5, as follows:

[SEQ ID No: 5]
TTTATCGGAGAGTTTGATCCTGGCTCAGGACGAACGCTGGCGGCGTGCC
TAATACATGCAAGTCGAGCGGACAGATGGGAGCTTGCTCCCTGATGTTA
GCGGCGGACGGGTGAGTAACACGTGGGTAACCTGCCTGTAAGACTGGGA
TAACTCCGGGAAACCGGGGCTAATACCGGATGGTTGTCTGAACCGCATG
GTTCAGACATAAAAGGTGGCTTCGGCTACCACTTACAGATGGACCCGCG
GCGCATTAGCTAGTTGGTGAGGTAACGGCTCACCAAGGCGACGATGCGT
AGCCGACCTGAGAGGGTGATCGGCCACACTGGGACTGAGACACGGCCCA
GACTCCTACGGGAGGCAGCAGTAGGGAATCTTCCGCAATGGACGAAAGT
CTGACGGAGCAACGCCGCGTGAGTGATGAAGGTTTTCGGATCGTAAAGC
TCTGTTGTTAGGGAAGAACAAGTGCCGTTCAAATAGGGCGGCACCTTGA
CGGTACCTAACCAGAAAGCCACGGCTAACTACGTGCCAGCAGCCGCGGT
AATACGTAGGTGGCAAGCGTTGTCCGGAATTATTGGGCGTAAAGGGCTC
GCAGGCGGTTTCTTAAGTCTGATGTGAAAGCCCCCGGCTCAACCGGGGA
GGGTCATTGGAAACTGGGGAACTTGAGTGCAGAAGAGGAGAGTGGAATT
CCACGTGTAGCGGTGAAATGCGTAGAGATGTGGAGGAACACCAGTGGCG
AAGGCGACTCTCTGGTCTGTAACTGACGCTGAGGAGCGAAAGCGTGGGG
AGCGAACAGGATTAGATACCCTGGTAGTCCACGCCGTAAACGATGAGTG
CTAAGTGTTAGGGGGTTTCCGCCCCTTAGTGCTGCAGCTAACGCATTAA
GCACTCCGCCTGGGGAGTACGGTCGCAAGACTGAAACTCAAAGGAATTG
ACGGGGGCCCGCACAAGCGGTGGAGCATGTGGTTTAATTCGAAGCAACG
CGAAGAACCTTACCAGGTCTTGACATCCTCTGACAATCCTAGAGATAGG
ACGTCCCCTTCGGGGGCAGAGTGACAGGTGGTGCATGGTTGTCGTCAGC
TCGTGTCGTGAGATGTTGGGTTAAGTCCCGCAACGAGCGCAACCCTTGA
TCTTAGTTGCCAGCATTCAGTTGGGCACTCTAAGGTGACTGCCGGTGAC
AAACCGGAGGAAGGTGGGGATGACGTCAAATCATCATGCCCCTTATGAC
CTGGGCTACACACGTGCTACAATGGACAGAACAAAGGGCAGCGAAACCG
CGAGGTTAAGCCAATCCCACAAATCTGTTCTCAGTTCGGATCGCAGTCT
GCAACTCGACTGCGTGAAGCTGGAATCGCTAGTAATCGCGGATCAGCAT
GCCGCGGTGAATACGTTCCCGGGCCTTGTACACACCGCCCGTCACACCA
CGAGAGTTTGTAACACCCGAAGTCGGTGAGGTAACCTTTATGGAGCCAG
CCGCCGAAGGTGGGACAGATGATTGGGGTGAAGTCGTAACAAGGTAGCC
GTATCGGAAGGTGCGGCTGGATCACCTCCTTTCTAA

Accordingly, preferably the bacterium comprises the nucleotide sequence substantially as set out in SEQ ID No: 5, or a variant or fragment thereof.

In another preferred embodiment, the bacterium may comprise 16S rDNA of NCIMB 43392, which is provided herein as SEQ ID No: 6, as follows:

[SEQ ID No: 6]
TTTATCGGAGAGTTTGATCCTGGCTCAGGACGAACGCTGGCGGCGTGCC
TAATACATGCAAGTCGAGCGGACAGATGGGAGCTTGCTCCCTGATGTTA
GCGGCGGACGGGTGAGTAACACGTGGGTAACCTGCCTGTAAGACTGGGA
TAACTCCGGGAAACCGGGGCTAATACCGGATGGTTGTCTGAACCGCATG
GTTCAGACATAAAAGGTGGCTTCGGCTACCACTTACAGATGGACCCGCG
GCGCATTAGCTAGTTGGTGAGGTAACGGCTCACCAAGGCGACGATGCGT
AGCCGACCTGAGAGGGTGATCGGCCACACTGGGACTGAGACACGGCCCA
GACTCCTACGGGAGGCAGCAGTAGGGAATCTTCCGCAATGGACGAAAGT
CTGACGGAGCAACGCCGCGTGAGTGATGAAGGTTTTCGGATCGTAAAGC
TCTGTTGTTAGGGAAGAACAAGTGCCGTTCAAATAGGGCGGCACCTTGA
CGGTACCTAACCAGAAAGCCACGGCTAACTACGTGCCAGCAGCCGCGGT
AATACGTAGGTGGCAAGCGTTGTCCGGAATTATTGGGCGTAAAGGGCTC
GCAGGCGGTTTCTTAAGTCTGATGTGAAAGCCCCCGGCTCAACCGGGGA
GGGTCATTGGAAACTGGGGAACTTGAGTGCAGAAGAGGAGAGTGGAATT
CCACGTGTAGCGGTGAAATGCGTAGAGATGTGGAGGAACACCAGTGGCG
AAGGCGACTCTCTGGTCTGTAACTGACGCTGAGGAGCGAAAGCGTGGGG
AGCGAACAGGATTAGATACCCTGGTAGTCCACGCCGTAAACGATGAGTG
CTAAGTGTTAGGGGGTTTCCGCCCCTTAGTGCTGCAGCTAACGCATTAA
GCACTCCGCCTGGGGAGTACGGTCGCAAGACTGAAACTCAAAGGAATTG
ACGGGGGCCCGCACAAGCGGTGGAGCATGTGGTTTAATTCGAAGCAACG
CGAAGAACCTTACCAGGTCTTGACATCCTCTGACAATCCTAGAGATAGG
ACGTCCCCTTCGGGGGCAGAGTGACAGGTGGTGCATGGTTGTCGTCAGC
TCGTGTCGTGAGATGTTGGGTTAAGTCCCGCAACGAGCGCAACCCTTGA
TCTTAGTTGCCAGCATTCAGTTGGGCACTCTAAGGTGACTGCCGGTGAC
AAACCGGAGGAAGGTGGGGATGACGTCAAATCATCATGCCCCTTATGAC
CTGGGCTACACACGTGCTACAATGGACAGAACAAAGGGCAGCGAAACCG
CGAGGTTAAGCCAATCCCACAAATCTGTTCTCAGTTCGGATCGCAGTCT
GCAACTCGACTGCGTGAAGCTGGAATCGCTAGTAATCGCGGATCAGCAT
GCCGCGGTGAATACGTTCCCGGGCCTTGTACACACCGCCCGTCACACCA
CGAGAGTTTGTAACACCCGAAGTCGGTGAGGTAACCTTTATGGAGCCAG
CCGCCGAAGGTGGGACAGATGATTGGGGTGAAGTCGTAACAAGGTAGCC
GTATCGGAAGGTGCGGCTGGATCACCTCCTTTCTAA

Accordingly, preferably the bacterium comprises the nucleotide sequence substantially as set out in SEQ ID No: 6, or a variant or fragment thereof.

In another preferred embodiment, the bacterium may comprise 16S rDNA of B. subtilis strain SG188, as follows:

[SEQ ID No: 29]
CTTTATCGGAGAGTTTGATCCTGGCTCAGGACGAACGCTGGCGGCGTGC
CTAATACATGCAAGTCGAGCGGACAGATGGGAGCTTGCTCCCTGATGTT
AGCGGCGGACGGGTGAGTAACACGTGGGTAACCTGCCTGTAAGACTGGG
ATAACTCCGGGAAACCGGGGCTAATACCGGATGGTTGTTTGAACCGCAT
GGTTCAAACATAAAAGGTGGCTTCGGCTACCACTTACAGATGGACCCGC
GGCGCATTAGCTAGTTGGTGAGGTAACGGCTCACCAAGGCAACGATGCG
TAGCCGACCTGAGAGGGTGATCGGCCACACTGGGACTGAGACACGGCCC
AGACTCCTACGGGAGGCAGCAGTAGGGAATCTTCCGCAATGGACGAAAG
TCTGACGGAGCAACGCCGCGTGAGTGATGAAGGTTTTCGGATCGTAAAG
CTCTGTTGTTAGGGAAGAACAAGTACCGTTCGAATAGGGCGGTACCTTG
ACGGTACCTAACCAGAAAGCCACGGCTAACTACGTGCCAGCAGCCGCGG
TAATACGTAGGTGGCAAGCGTTGTCCGGAATTATTGGGCGTAAAGGGCT
CGCAGGCGGTTTCTTAAGTCTGATGTGAAAGCCCCCGGCTCAACCGGGG
AGGGTCATTGGAAACTGGGGAACTTGAGTGCAGAAGAGGAGAGTGGAAT
TCCACGTGTAGCGGTGAAATGCGTAGAGATGTGGAGGAACACCAGTGGC
GAAGGCGACTCTCTGGTCTGTAACTGACGCTGAGGAGCGAAAGCGTGGG
GAGCGAACAGGATTAGATACCCTGGTAGTCCACGCCGTAAACGATGAGT
GCTAAGTGTTAGGGGGTTTCCGCCCCTTAGTGCTGCAGCTAACGCATTA
AGCACTCCGCCTGGGGAGTACGGTCGCAAGACTGAAACTCAAAGGAATT
GACGGGGGCCCGCACAAGCGGTGGAGCATGTGGTTTAATTCGAAGCAAC
GCGAAGAACCTTACCAGGTCTTGACATCCTCTGACAATCCTAGAGATAG
GACGTCCCCTTCGGGGGCAGAGTGACAGGTGGTGCATGGTTGTCGTCAG
CTCGTGTCGTGAGATGTTGGGTTAAGTCCCGCAACGAGCGCAACCCTTG
ATCTTAGTTGCCAGCATTCAGTTGGGCACTCTAAGGTGACTGCCGGTGA
CAAACCGGAGGAAGGTGGGGATGACGTCAAATCATCATGCCCCTTATGA
CCTGGGCTACACACGTGCTACAATGGACAGAACAAAGGGCAGCGAAACC
GCGAGGTTAAGCCAATCCCACAAATCTGTTCTCAGTTCGGATCGCAGTC
TGCAACTCGACTGCGTGAAGCTGGAATCGCTAGTAATCGCGGATCAGCA
TGCCGCGGTGAATACGTTCCCGGGCCTTGTACACACCGCCCGTCACACC
ACGAGAGTTTGTAACACCCGAAGTCGGTGAGGTAACCTTTTAGGAGCCA
GCCGCCGAAGGTGGGACAGATGATTGGGGTGAAGTCGTAACAAGGTAGC
CGTATCGGAAGGTGCGGCTGGATCACCTCCTTTCTA

Accordingly, preferably the bacterium comprises the nucleotide sequence substantially as set out in SEQ ID No: 29, or a variant or fragment thereof.

In one embodiment, the bacterium may comprise one or more of the nucleotide sequences selected from the group consisting of: SEQ ID No: 1 to 6, 28 and 29, or variants or fragmentsthereof.

In one embodiment, the bacterium may comprise two or more of the nucleotide sequences selected from the group consisting of: SEQ ID No: 1 to 6, 28 and 29, or variants or fragmentsthereof.

In one embodiment, the bacterium may comprise three or more of the nucleotide sequences selected from the group consisting of: SEQ ID No: 1 to 6, 28 and 29, or variants or fragments thereof.

In one embodiment, the bacterium may comprise four or more of the nucleotide sequences selected from the group consisting of: SEQ ID No: 1 to 6, 28 and 29, or variants or fragmentsthereof.

In one embodiment, the bacterium may comprise five or more of the nucleotide sequences selected from the group consisting of: SEQ ID No: 1 to 6, 28 and 29, or variants or fragmentsthereof.

In one embodiment, the bacterium may comprise six or more of the nucleotide sequences selected from the group consisting of: SEQ ID No: 1 to 6, 28 and 29, or variants or fragmentsthereof.

In one embodiment, the bacterium may comprise the nucleotide sequences selected from the group consisting of: SEQ ID No: 1 to 6, 28 and 29, or variants or fragments thereof.

Homologues and paralogues of any of the sequences described herein across different species are also envisaged as being part of the invention.

Preferably, the live or dead bacterial spore, live or dead vegetative bacterium, the extracellular material produced by the live cell or the disrupted bacterial cell homogenate produces or comprises at least one sporulene family member and at least one non-ribosomal peptide.

For example, in one embodiment, the live or dead bacterial spore, live or dead vegetative bacterium, the extracellular material produced by the live cell or the disrupted bacterial cell homogenate further produces or comprises the squalene cyclase gene, sqhC, and at least one lipopeptide selected from the group consisting of a member of the Fengycin family; a member of the Surfactin family; and a member of the Iturin family.

Preferably, the live spore, dead spore, or live vegetative cell or dead cell, or the extracellular material produced by the live cell or the disrupted cell homogenate therefrom, in accordance with the first or second aspect, comprises an antiviral composition. Preferably, the live or dead bacterial spore, the live or dead vegetative bacterium, the extracellular material produced by the live cell, or the disrupted bacterial cell homogenate exhibits anti-viral properties and/or innate immunity stimulation properties.

The inventors believe that it is the antiviral/innate immunity stimulation composition which is responsible for the surprising antiviral activity exhibited.

Hence, in a third aspect, there is provided an antiviral and/or innate immune stimulation composition comprising at least one sporulene family member and/or a lipopeptide selected from the group consisting of a member of the Surfactin family, a member of the Iturin family and a member of the Fengycin family, or an active derivative of any of these lipopeptides, for use in in treating, preventing or ameliorating a virus infection.

The composition may further comprise a glycolipid or further lipopeptide as defined in the first aspect.

In a fourth aspect of the invention, there is provided a method of treating a virus infection, the method comprising administering, or having administered, to a patient in need of such treatment, a therapeutically effective amount of at least one sporulene family member and/or a lipopeptide selected from the group consisting of a member of the Surfactin family, a member of the Iturin family and a member of the Fengycin family, or an active derivative of any of these lipopeptides.

The method may further comprise administering, or having administered, a glycolipid or further lipopeptide as defined in the first aspect.

In a fifth aspect of the invention, there is provided the live or dead bacterial spore, a live or dead vegetative bacterium, extracellular material produced by the live cell, or a disrupted bacterial cell homogenate, as defined in the first aspect, or the antiviral and/or innate immune stimulation composition as defined in the third aspect, for use as a foodstuff or dietary supplement.

In a sixth aspect of the invention, there is provided a dietary supplement or foodstuff comprising the live or dead bacterial spore, alive or dead vegetative bacterium, extracellular material produced by the live cell, or a disrupted bacterial cell homogenate as defined in the first aspect, or the antiviral and/or innate immune stimulation composition as defined in the third aspect, and optionally one or more food grade ingredients.

The sporulene family member, respiratory virus, surfactin family member, member of the Iturin family, member of the Fengycin family, and use may be as defined in the first aspect. Preferably, the virus being treated or prevented (i.e., vaccinated) is a respiratory virus, such as a virus selected from the group consisting of: Respiratory syncytial virus (RSV), Coronavirus and Rhinovirus.

Preferably, the respiratory virus is a Coronavirus. More preferably, the Coronavirus is selected from MERS, SARS-CoV1 and SARS-CoV2. Most preferably, the respiratory virus is SARS-CoV2.

In some embodiments, the composition may be a probiotic, for example, when delivered in combination with a live spore or a live vegetative cell. The foodstuff may be a beverage. In another embodiment, the foodstuff may be a medical foodstuff, or “medical food”. The skilled person would understand that the term “medical food” refers to a foodstuff that has a health claim associated with it.

In some embodiments, the agents and compositions of the invention may be administered in the form of a foodstuff or dietary supplement, for example a probiotic. The foodstuff may be a beverage. In another embodiment, the foodstuff may be a medical foodstuff, or “medical food”. The skilled person would understand that the term “medical food” refers to a foodstuff that has a health claim associated with it.

When the preparation of the invention is in the form of a food supplement, it can be in a form for separate administration, such as a capsule, a tablet, a powder or a similar form, containing preferably a unit dose of the microorganisms, containing 10²-10¹⁵ cells/dose, preferably 10⁸-10¹¹ cells/dose (where cells are either in the form of vegetative cells or spores or a mixture thereof).

The food supplement can also be in the form of a powder or a similar form, which is added to, or mixed with, a suitable food (composition) or a suitable liquid or solid carrier, for the preparation of a food which is ready for consumption.

For instance, the food supplement can be in the form of a dried powder, which is reconstituted using a suitable liquid, such as water, oral rehydration solution, milk, fruit juice, or similar drinkable liquids. It can also be in the form of a powder which is mixed with solid foods, or foods with a high water-content, such as fermented milk products, for example yoghurt.

The composition of the invention can also be in the form of a food which is ready for consumption. Such a food can for instance be prepared by adding a supplement of the invention as described above to a food or food base known per se; adding the microorganisms (separately or as a mixture) in the amounts required for administration to a food or food base known per se; or by cultivating the required bacteria in a food medium until a food containing the amount of bacteria required for administration is obtained. The food medium is preferably such that it already forms part of the food, or will form part of the food after fermentation.

In this respect, the food or food base can be either fermented or non-fermented. The composition of the invention can be foods for oral consumption, for instance a total food or an infant formula.

The composition can further contain prebiotic compounds, in particular fibers that lead to the production butyrate/butyric acid, propionate/propionic acid or acetate/acetic acid upon fermentation; nitrogen donors such as proteins; and specific vitamins, minerals and/or trace elements. With respect to the latter, and as can be seen from the examples, the presence of increased or moderately high amounts of vitamin A, K, B₁₂, biotin, Mg, Ca and Zn can be advantageous, as can the presence of folic acid in preparations intended for the treatment of chronic diarrhea.

The food supplement may further comprise fibers e.g., an amount of at least 0.5 g fiber per 100 g of the total preparation.

As the fibers, the preparation preferably contains a resistant starch or another butyrate generator, as well as a suitable propionate generator such as gums or soy polysaccharides, in the amounts indicated above. Short-chain fatty acids such as butyric acid and propionic acid can also be used as such, preferably in a suitably encapsulated form, or as a physiological equivalent thereof, such as sodium propionate, in an amount of at least 0.1 g per 100 g of the total composition.

Nitrogen, vitamins, minerals and trace elements may also be included e.g., in form of yeast extract.

The composition of the invention can further contain one or more substances that inhibit bacterial adhesion to the epithelial wall of the gastrointestinal tract. Preferably, these compounds are selected from lectins, glycoproteins, mannans, glucans, chitosan and/or derivatives thereof, charged proteins, charged carbohydrates, sialylated compounds and/or adhesion-inhibiting immunoglobulins, galacto-oliogasaccharides, as well as modified carbohydrates and modified chi-tin, the latter in amounts of 1-10% w/v, preferably 2-5% w/v of the composition.

Preferred adherence-inhibiting substances are chitosan, carob flour, as well as extracts which are rich in condensed tannin and tannin-derivatives, such as cranberry extract; the amount of tannin in the final product preferably being 10-600 μg/ml. The composition, in particular if the composition is in the form of a total food, may also contain peptides and/or proteins, in particular proteins that are rich in glutamate and glutamine, lipids, carbohydrates, vitamins, minerals and trace elements. The use of glutamine/glutamate precursors, in amounts corresponding to 0.6-3 g glutamine/100 g product, as well as of small polypeptides that have a high content of glutamines, is preferred. Alternatively, proteins that are rich in glutamine, such as milk proteins, wheat proteins or hydrolysates thereof, can be added.

In one preferred embodiment the composition further comprise glucosamine. Glucosamine is typically included in amounts corresponding to a daily intake in the range of 10-2000 mg, e.g., in the range of 100-2000 mg, e.g., in the range of 250-1500 mg e.g., in the range of 500-1000 mg. In particular preferred embodiments the compositions of the invention is formulated in unit dosage forms, where 1-5 unit dosage forms correspond to a daily dosage of 10² to 10¹⁵, preferably 10⁶ to 10¹², in particular 10⁸ to 10¹¹, Bacillus cells and glucosamine in amounts in the range of 10-2000 mg, e.g., in the range of 100-2000 mg, e.g. in the range of 250-1500 mg e.g., in the range of 500-1000 mg. The glucosamine may be glucosamine hydrochloride or glucosamine phosphate. The skilled person would understand that glucosamine may also be referred to as chitosamine.

The compositions of the invention may be lactose-free. In some embodiments the compositions have a high osmolality (preferably less than 400 mosm/l, more preferably less than 300 mosm/1), in other embodiments the compositions have a lower osmolality e.g. as the compositions disclosed in https://www.ncbi.nlm.nih.gov/pmc/articles/PMC1717650/. In some embodiments the compositions of the invention are kosher, vegetarian or vegan.

In some embodiments the composition of the invention is a pharmaceutical composition comprising a strain of the invention and one or more pharmaceutically acceptable excipients.

The inventors have prepared novel foods, food ingredients, dietary supplements, dietary supplement ingredients, medical foods, foods for special medical purposes, foods for specified health use, foods for special dietary use, health foods, Complementary Medicines Natural Health Products, Natural Health formulations, Natural Health ingredients, and pharmaceutical products, pharmaceutical preparations, pharmaceutical formulations, and pharmaceutical ingredients comprising a bacteria, for example one of the various B. amyloliquefaciens and B. subtilis strains or any combination of such strains.

Accordingly, there is provided a composition comprising a live or dead spore, or a live or dead vegetative cell, or mixture thereof, of one or more B. amyloliquefaciens strains and/or one or more B. subtilis strains, or extracellular material produced by the live cell, or disrupted cell homogenate; and, one or more food grade ingredients preferably selected among: carrier or vehicle, fillers, stabilizers, nutrients, flavorings, colorings.

The carriers or vehicles are selected among any food grade materials that are inert under the conditions applied during storing and use. Examples of carriers or vehicles includes minerals, such as CaCO₃, NaCl, KCl, CaHPO₄; polymers such as natural or modified starch, pectin, cellulose; sugar such as lactose, sucrose or glucose; flour and skimmed milk powder.

The fillers are selected among ingredients that are inert under the conditions applied. Fillers are typically added to the compositions of the invention to secure that the composition obtain the desired volume.

The stabilizers are selected among food grade ingredients having the ability to stabilize and/or protect the one or more B. amyloliquefaciens strains and/or one or more B. subtilis strains, during production and/or storage. Examples of suitable stabilizers include ascorbic acid and vitamin E.

The nutrients can in principle be selected among any nutrients, provided that the composition does not support growth of the one or more B. amyloliquefaciens strains and/or one or more B. subtilis strains. Typically this means that the composition is dry or at least that the water activity is so low that microbial growth is prevented. Example so of nutrients includes minerals, vitamins, sugars, proteins, milk or fractions thereof including milk powders, flour, honey and juice.

The flavorings and colorants are selected among food grade flavorings and colorants as known in the area.

The composition may be a food, food ingredient, dietary supplement, dietary supplement ingredient, medical food, food for special medical purposes, food for specified health use, food for special dietary use, health food, Complementary Medicine; Natural Health Product, Natural Health formulation, Natural Health ingredient, pharmaceutical product, pharmaceutical preparation, pharmaceutical formulation, or a pharmaceutical ingredient.

The composition may be a probiotic composition comprising or consisting of live cells, or spores, or compounds originating from the cells or spores. The composition may comprise one or more food grade ingredients selected from fillers and stabilizing agents. The composition may be provided in a unit dosage formulation, such as a capsule, tablet or sachet. Each unit dosage formulation may comprise 10⁸ to 10¹⁰ CFU of the one or more microorganisms. The composition may be a food composition, comprising at least one nutrient and/or vitamin in addition to the one or more microbial strains. The food composition may comprise a CFU count corresponding to 10⁸ to 10¹⁰ CFU per serving.

The one or more microorganisms may be provided in lyophilized or spray-dried form. The composition may further comprise glucosamine. The composition may comprise glucosamine corresponding to a daily dosage of 10-2000 mg, optionally in the range of 100-2000 mg, or in the range of 250-1500 mg, or in the range of 500-1000 mg. The glucosamine may be glucosamine hydrochloride or glucosamine phosphate.

Preferably, the agents and formulations of the invention (i.e., (i) the live or dead bacterial spore, (ii) the live or dead vegetative bacterium, (iii) the extracellular material produced by the live cell, (iv) the disrupted bacterial cell homogenate, or (v) the composition of the invention) exhibit anti-viral properties and innate immunity stimulation properties. Thus, by the term “antiviral”, it can mean both antiviral and/or innate immunity stimulation properties.

It will be appreciated that the antiviral agents and formulations according to the invention may be used in a monotherapy (i.e., the sole use of (i) live or dead bacterial spore, (ii) a live or dead vegetative bacterium, (iii) extracellular material produced by the live cell, (iv) a disrupted bacterial cell homogenate, or (v) antiviral composition of the invention), for treating, ameliorating or preventing a respiratory virus infection, most preferably SARS-CoV2. Alternatively, such antiviral agents and formulations according to the invention may be used as an adjunct to, or in combination with, known therapies for treating, ameliorating, or preventing respiratory virus infections, for example SARS-CoV2. The inventors believe that the antiviral agents and formulations according to the invention may advantageously act as adjuvants when used in combination with existing virus vaccines. The antiviral agents and formulations may act as innate immunity stimulant, which complements the action of existing vaccines, which induce an adaptive immune response in a subject treated with the vaccine. Thus, in one embodiment, the agents and formulations according to the invention may be used in combination with existing virus vaccines, preferably a coronavirus vaccine or influenza vaccine.

The agents and formulations according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

The agents and formulations of the invention may be used in a number of ways. For instance, oral administration may be required, in which case the agents may be contained within a composition that may, for example, be ingested orally in the form of a tablet, capsule or liquid, which may include delivery of a composition present in food or a beverage.

Antiviral compositions and formulations of the invention are preferably administered by mucosal delivery. Antiviral compositions and formulations of the invention may be preferably administered by inhalation (e.g., intranasally).

Therefore, preferably the live or dead bacterial spore, live or dead vegetative bacterium, extracellular material produced by the live cell, or disrupted bacterial cell homogenate for use in treating the viral infection, is applied nasally. Nasal application may comprise application by a spray, mist, droplet, cream, gel or injection. In another embodiment, it is preferred that a live bacterial spore is used to treat a viral infection, and is applied nasally, preferably by spray, mist, droplet, cream, gel or injection. In one embodiment, it is preferred that a dead bacterial spore is used to treat a viral infection, and is applied nasally, preferably by spray, mist, droplet, cream, gel or injection. The dosage used for nasal administration may be 10² to 10¹⁵, preferably 10⁶ or 10⁸ to 10¹², in particular 10⁸ to 10¹⁰, bacterial cells (either in the form of vegetative cells or spores or a mixture thereof).

Alternatively, the live or dead bacterial spore, live or dead vegetative bacterium, extracellular material produced by the live cell, or disrupted bacterial cell homogenate may be applied sublingually. Sublingual application may comprise application by a wafer (e.g., a buccal wafer) or fast-dissolving film.

Compositions may also be formulated for topical use. For instance, creams or ointments may be applied to the skin. Alternatively, compositions may be delivered by sub-lingual administration. Preferably, the compositions are formulated for mucosal application.

Agents and formulations according to the invention may also be incorporated within a slow- or delayed-release device. Such devices may, for example, be inserted on or under the skin, and the medicament may be released over weeks or even months. The device may be located at least adjacent to the treatment site. Such devices may be particularly advantageous when long-term treatment with agents used according to the invention is required and which would normally require frequent administration (e.g., at least daily administration).

In a preferred embodiment, agents and formulations according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the agents and formulations that is required is determined by its biological activity and bioavailability, which in turn depends on the mode of administration, the physiochemical properties of the agents and formulations, and whether they are being used as a monotherapy or in a combined therapy. The frequency of administration will also be influenced by the half-life of the agents and formulations within the subject being treated. Optimal dosages to be administered may be determined by those skilled in the art, and will vary with the particular agents and formulations in use, the strength of the pharmaceutical composition, the mode of administration, and the advancement of the viral infection. Additional factors depending on the particular subject being treated will result in a need to adjust dosages, including subject age, weight, gender, diet, and time of administration.

Generally, a daily dose of between 0.001 μg/kg of body weight and 10 mg/kg of body weight of agents, antiviral (and/or innate immunity stimulation) compositions or formulation according to the invention may be used for treating, ameliorating, or preventing respiratory infection, depending upon which agents and formulations is used. More preferably, the daily dose is between 0.01 g/kg of body weight and 1 mg/kg of body weight, more preferably between 0.1 g/kg and 100 g/kg body weight, and most preferably between approximately 0.1 g/kg and log/kg body weight.

Agents or compositions according to the invention may for instance be characterized in that they contain 10² to 10¹⁵, preferably 10⁶ or 10⁸ to 10¹², in particular 10⁸ to 10¹⁰, bacterial cells (either in the form of vegetative cells or spores or a mixture thereof). Reference value is a unit of administration, for instance a tablet, a capsule or a sachet. The compositions may be prepared for oral administration. The bacterial cells are suitably lyophilized or spray dried.

In the case of the food composition, it may be provided that the composition contains 10² to 10¹⁵, preferably 10⁶ to 10⁹, in particular 10⁷ to 10⁹, bacterial cells (either in the form of vegetative cells or spores or a mixture thereof). Reference value is a unit of administration, for instance a packing unit of a food material to be sold to an end user. The physiologically tolerated carrier will normally be a food material, which in particular is selected from the group comprising “milk products, fermented milk products, milk, yogurt, cheese, cereals, muesli bars, and children's food preparations”.

The agents and formulations may be administered before, during or after the onset of the viral infection. Daily doses may be given as a single administration (e.g., a single daily injection, or oral dose). Alternatively, the agents and formulations may require administration two or more times during a day or one or more times a week, or one or more times a month. As an example, the agents and formulations may be administered as two (or more depending upon the severity of the viral infection being treated) daily doses of between 0.07 g and 700 mg (i.e., assuming a body weight of 70 kg). A patient receiving treatment may take a first dose upon waking and then a second dose in the evening (if on a two-dose regime) or at 3- or 4-hourly intervals thereafter. Alternatively, a slow release device may be used to provide optimal doses of agents and formulations according to the invention to a patient without the need to administer repeated doses.

Known procedures, such as those conventionally employed by the pharmaceutical industry (e.g., in vivo experimentation, clinical trials, etc.), may be used to form specific formulations of the agents and formulations according to the invention and precise therapeutic regimes (such as daily doses of the agents and formulations and the frequency of administration).

The invention also provides in a seventh aspect, a pharmaceutical composition comprising the live or dead bacterial spore, alive or dead vegetative bacterium, extracellular material produced by the live cell, a disrupted bacterial cell homogenate as defined in the first aspect, or the antiviral composition for use as defined in the third aspect, and a pharmaceutically acceptable vehicle or carrier.

In an eighth aspect, there is also provided a process for making the composition according to the seventh aspect, the process comprising combining a therapeutically effective amount of the live or dead bacterial spore, alive or dead vegetative bacterium, extracellular material produced by the live cell, a disrupted bacterial cell homogenate as defined in the first aspect, or the antiviral composition as defined in the third aspect, with a pharmaceutically acceptable vehicle or carrier.

A “subject” may be a vertebrate, mammal, or domestic animal. Hence, medicaments according to the invention may be used to treat any mammal, for example livestock (e.g., a horse), pets, or may be used in other veterinary applications. Most preferably, the subject is a human being.

A “therapeutically effective amount” of the live or dead bacterial spore, the live or dead vegetative bacterium, extracellular material produced by the live cell, or the disrupted bacterial cell homogenate according to the first aspect, or the antiviral composition according to the third aspect is any amount which, when administered to a subject, is the amount of the active component that is needed to treat the infection, or produce the desired effect.

For example, the therapeutically effective amount may be from about 0.001 μg to about 1 mg, and preferably from about 0.01 μg to about 100 μg. It is preferred that the amount of agent is an amount from about 0.1 μg to about 10 μg, and most preferably from about 0.5 μg to about 5 μg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example, calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The agents and compositions of the invention may be administered sub-lingually, for example in the form of a slow release film, wafer or caplet.

All features described herein (including any accompanying claims, abstract and drawings), and/or all of the steps of any method or process so disclosed, may be combined with any of the above aspects in any combination, except combinations where at least some of such features and/or steps are mutually exclusive.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-29 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, in those of SEQ ID Nos: 1 to 29 that are amino acid sequences.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

For a better understanding of the invention, and to show how embodiments of the same may be carried into effect, reference will now be made, by way of example, to the accompanying Figures, in which:—

FIG. 1 shows the ability of Bacillus spp. spores to protect against viral infection. Mice were dosed with 2 nasal doses (2×10⁹/dose) of killed Bacillus spp. spores (Spores^{K}) and challenged on day 42 with 10 LD50 of H5N2 (A) and (B). Survival rates of mice challenged with H5N2 is shown in (C) and body weights (D), 2 nasal doses of killed Bacillus spores (Spores^{K}) provide 100% protection to live H5N2 Protection is equivalent to that of dosing with inactivated influenza virus (i.e., a flu vaccine) and exemplified by a control group (iNiB) consisting of 2 nasal doses of 0.5 μg HA of NIBRG-14 (A/Aquatic Bird/Korea) that likewise provided 100% protection.

FIG. 2 shows the adjuvant effect of Bacillus spp. spores (killed Bacillus spores, Spores^{K}) when used in combination with inactivated H5N1 virions (defined as μg HA). The experimental design is shown in (A) and (B). Survival rates of mice (n=5/gp) challenged with influenza, H5N2 (A/Aquatic Bird/Korea) (10 LD₅₀) is shown in (C) and body weights (D). Naive animals and those dosed with 0.02 μg of HA showed no survival following challenge. Spores^{K} provided 60% protection to challenge. 0.5 μg of virion HA provided protection (80%) but this was increased to 100% when mixed with spores (2×10⁹ CFU), as shown in (C). Body weight was also monitored throughout (D).

FIG. 3 shows the survival rates of mice challenged with H5N2 (A/Aquatic Bird/Korea) (10 LD₅₀), having been dosed with 2 nasal doses (2×10⁹/dose) of killed Bacillus spp. spores (Spores^{K}). (A) and (B) show the experimental design. Survival rates area shown in (C) and body weight in (D).

FIG. 4 shows the dose-dependent survival rates of mice treated with varying amounts of killed Bacillus spp. spores (Spores^{K}). Study design is shown in ((A). Mice treated with 5×10⁸ and 2×10⁹ were observed to have 100% survival 14 days post-challenge with 5 LD₅₀ H5N2 (C). Body weights are shown in (D).

FIG. 5 the adjuvant effect of wild type spores and the involvement of SqhC in this response. In the first approach wild type (Group 1) or sqhC (Group 2) isogenic spores were killed and injected into mice by the subcutaneous route (days 1, 15 and 36). Simultaneously inactivated H5N1 virions were used to dose the same mice by the intranasal route on days 1, 15 and 36). In a second approach either wild type (Group 3) or sahC (Group 4) killed spores were mixed and adsorbed with inactivated H5N1 virions and then used to dose mice (day 1, 15 and 36) by the intra-nasal route. Resulting concentrations of secretory IgA (SIgA) in saliva in of treated mice are shown.

FIG. 6 shows an SEC Chromatogram of an ammonium sulphate precipitate of material secreted by a Bacillus spp. SG277. Four fractions (Sec0, Sec1; Sec2 and Sec3) were identified as Fengycin, Surfactin, Iturin and Chlorotetaine, respectively.

FIG. 7 shows the adjuvant effect of killed Bacillus spp. spores (Spores^{K}) when used in combination with a H5N1 inactivated virions. The study design is shown in (A) and survival of mice shown in (B). Killed spores provided about 50% protection to H5N2 challenge (A/Aquatic Bird/Korea) (10 LD₅₀). Using H5N1 (iNiBRG14 clade 1) virions defined in HA as 0.02 μg or 0.1 μg protection was absent (0.02 μg) or low (0.1 μg) When mixed with killed spores protection was 100%, thus demonstrating the adjuvant effect of the spores.

FIG. 8 shows the stability of Bovine Serum Albumin (BSA; 0.1 mg/ml) versus log concentration of bile acids (lithocholate, hyodeoxycholate, deoxycholate), surfactin and 277-SEC. The methanol (MeOH) baseline was included in the graph to compensate for methanol interference in the assay. All assays were performed in 1×PBS buffer in the presence of reporter fluorescence dye-SYPRO-Orange.

FIG. 9 shows the outcome of dosing hACE transgenic mice with 2 intra-nasal doses (day 1 and 14; 2×10⁹ CFU/dose) of killed Bacillus spp. spores (Spores^{K}). Four days after the last dose animals are challenged with SARSoCov2 (105 PFU). 4 days later key organs and tissues are recovered and examined for viral burden either as PFU (plaque forming units) or genomic copies (by qPCR).

FIG. 10 shows the antigen (spike)-specific antibody responses in mice immunised with recombinant SARS-CoV-2 Spike protein, and boosted nasally with either PBS buffer (rSp), or purified spores of B. subtilis strain PY79 (rSp+PY79), in serum (IgG) (Panel A), saliva (SIgA) (Panel B) and lungs (secretory IgA, SIgA) (Panel C).

FIG. 11 shows the survival rates (Panel A), body weight (Panel B) and disease score (Panel C) of mice challenged intra-nasally with SARS-CoV-2 (5×10³ PFU/50 ml/dose), and treated with three weekly intra-nasal doses of heat inactivated B. subtilis spores (SPOR-COV) at 1.5×10⁹ CFU/dose (30 ml/dose).

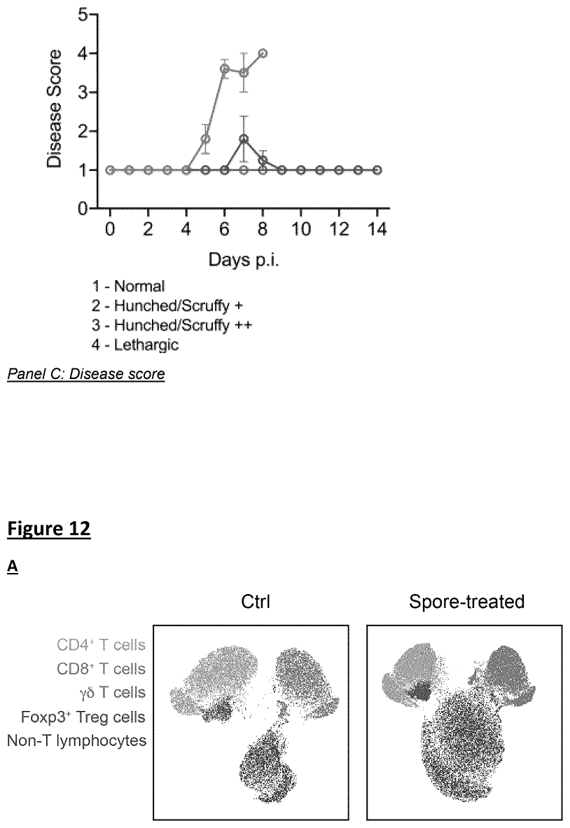

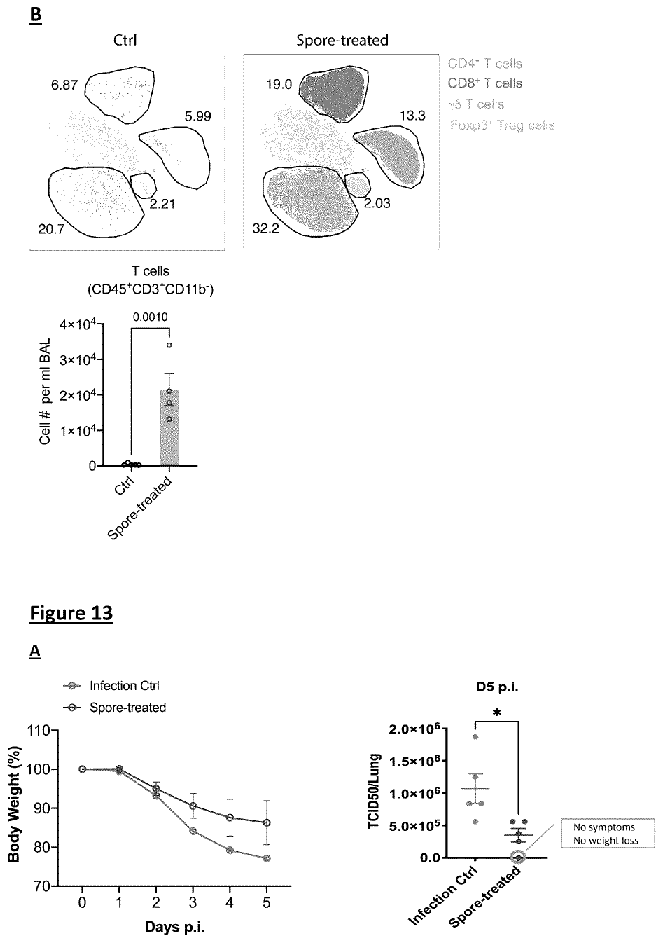

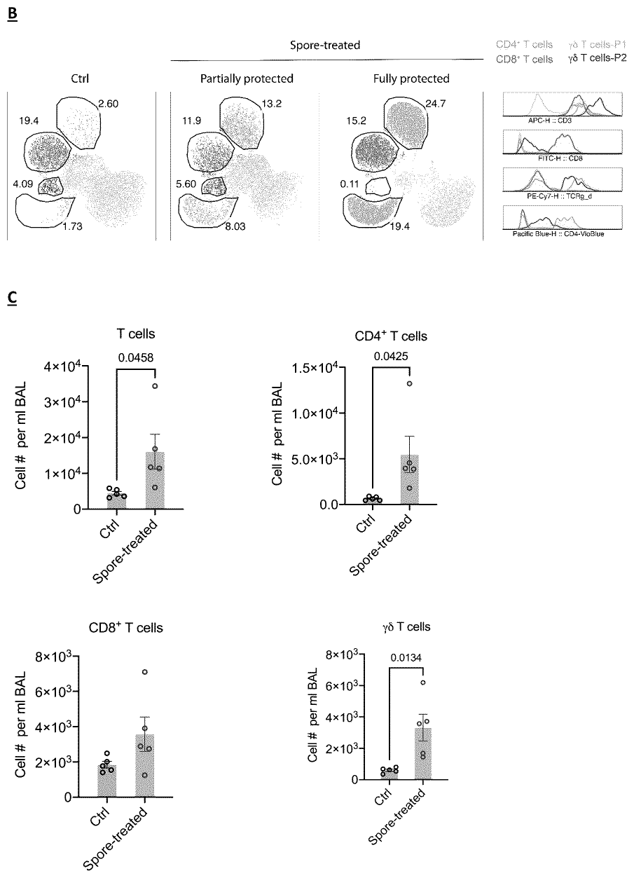

FIG. 12 shows the T cell responses in lung draining lymph node (Panel A) and lung alveolar space (Panel B) in mice treated with three weekly intra-nasal doses of 1.5×10⁹ CFU/30 μl of heat-killed spores (SPOR-COV).

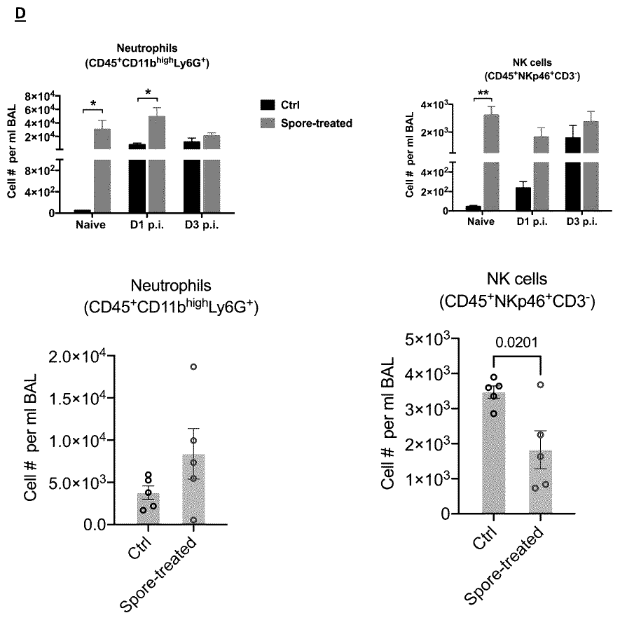

FIG. 13 illustrates the effect of mice treated with three weekly intra-nasal doses of 1.5×10⁹ CFU/30 μl of heat-killed spores (SPOR-COV) and challenged with 1×10² PFU/50 μl of H1N1-PR8 virus at seven days post the last dose of spores. (Panel A) shows body weight and virus load in the lungs following H1N1 infection. (Panel B) and (Panel C) illustrate T cell recruitment, and (Panel D) illustrates neutrophil and natural killer (NK) cell recruitment, into lung alveolar space following spore pre-treatment and H1N1 infection.

### EXAMPLES

The inventors first set out to determine the ability of spore-forming bacteria to protect against respiratory virus infections and to identify the factors produced by the bacteria that are associated with this protection. Based on their findings, they believe they have developed a highly effective nasally-administrable antiviral agent, for preventing or treating viral infections, such as Coronavirus.

### Materials and Methods

### H5N2 Survival Studies (Figures, 1-4 and 7)

Groups of mice (Balb/c) were housed in climate control secure facilities in standard animal cages in biosafety level 3 facilities. After 2 weeks of acclimatization animals were labelled and assigned to groups. Animals were anesthetized and dosed nasally (intra-nasal) using a pipette tip (max 20 microlitres/nostril) with spores or virions as described in the figures and below.

Dosing was always on day 0 and day 14. On day 42 animals were anesthetized and challenged with live H5N2 virus (A/Aquatic Bird/Korea (H5N2). Two challenge doses were used, either 5 LD₅₀ or 10 LD₅₀ as indicated in the text and legends. The HA protein of A/Aquatic Bird/Korea is 92-93% conserved with HA proteins of NIBRG-14 (H5N1). Mice were then monitored daily for clinical signs of influenza infection and body weight recorded daily.

The following suspensions were dosed (i.n.) to mice:
PBS—phosphate buffer saline and used as a control (naive).
Spores^{K}—spore suspension of B. subtilis that had been autoclaved (121° C., 15 psi, 30 min).

NIBRG-14—whole inactivated (formalin) H5N1 (NIBRG-14) virus defined in μg of HA (haemagglutinin). NIBRG-14 is A/Vietnam/1194/2004 (clade 1) and was obtained from the National Institute of Standards and Control (NIBSC), UK. NIBRG-14 was cultured from eggs as described elsewhere (Song et al 2012). HA concentration was determined by SRD (single radial immunodiffusion) assay using a standard and specific sheep antiserum (NIBSC, UK). A suspension of NIBRG-14 at 60 μg/ml(HA) was used as a working stock.

In some examples (in FIG. 2, and FIG. 7) inactivated NIBRG-14 virions were co-administered nasally with Spores^{K}. In this case the NIBRG-14 and Spores^{K} samples were mixed and co-incubated for 30 min at RT with gentle agitation in 0.1M PBS (pH 7.2) buffer. Spores then washed 1× before suspension in 0.01M PBS (pH7.2) buffer and dosing.

### Sqhcknockout Study (FIG. 5)

Groups of mice were dosed with wild type PY79 B. subtilis spores or an isogenic derivative (SG768 sqhC::kan trpC2) that carries the sqhCmutation of Bosak et al. [1]. In both cases spores were killed by autoclaving. Four groups of animals were used as well as naive. Each group of Balb/c mice was 6 animals. Dosing regime in all cases was day 1, 15 and 36. Each dose of spores used was 1×10⁹ autoclaved spores by the nasal route and 1×10⁸ by the parenteral (s.c.) route. Virus used was ˜1-1.2 μg HA of H5N1 (formaldehyde-inactivated Influenza A/Vietnam/1194/2004; NIBRG-14) which was dosed to animals alone or adsorbed to spores.

For adsorption, H5N1 was incubated for 30 min at RT with gentle agitation in 0.1M PBS (pH 7.2) buffer. Spores then washed 1× before suspension in 0.01M PBS (pH7.2) buffer and dosing.
Group 1: mice dosed sub-cut with autoclaved sqhC spores (1×10⁸) and intra-nasally with H5N1 virus (˜1-1.2 μg).
Group 2: mice dosed sub-cut with autoclaved PY79 spores (1×10⁸) and intra-nasally with H5N1 virus (˜1-1.2 μg).
Group 3: mice dosed intra-nasally with autoclaved sqhC spores (1×10⁹) adsorbed with H5N1(˜1-1.2 μg).
Group 4: mice dosed intra-nasally with autoclaved PY79 spores (1×10⁹) adsorbed with H5N1(˜1-1.2 μg).

Control groups were Naive and mice dosed with H5N1 (nasally).

Virus (H5N1)-specific SIgA was determined by ELISA.

### Thermal ShiftAssays (FIG. 8)

Thermal shift assay is the technique that employs a fluorescent dye SYPRO-Orange (Sigma 5000× dissolved in DMSO) that, due to its amphipathic nature, binds to the hydrophobic amino acids that are being exposed to the aqueous solution upon protein folding. The dye can be excited once it is complexed with hydrophobic residues with wavelength λₑₓ=480 nm to emit an easily detectable light signal at λₑₘ=568 nm. The reactions for the experiments were prepared as master mixes, with protein final concentration of 0.1 mg/ml, SYPRO-Orange dilution kept at lox and PBS at 1×. The final volume of each reaction in the tube was 30 μL. The samples were melted in a real-time PCR machine (StepOne Plus Applied Biosystems), where they were equilibrated before the melt at 4° C. for 1 hour and then melted at 1% temperature increment (corresponding to −1° C./min) until the temperature of 99° C. The recording was set to read the fluorescence from four recording emission channels named after the dyes they are often deployed to work with, namely ROX, FAM, VIC and TAMRA, as the emission spectrum of SYPRO—Orange overlaps with the emission spectra of these dyes. The reactions were run in technical triplicates. All the experiments were performed alongside a protein negative control (lox SYPRO-Orange and the buffer only). Surfactin and bile acids were dissolved in final assay concentrations of 4 mg/ml→0.015 mg/ml in methanol (50%→0.2%, two-fold difference each step). 277 SEC dilutions were prepared in 1×PBS. The concentration of BSA was 0.1 mg/ml and SYPRO-Orange dilution was kept at lox. DMSO was present in the assay at 0.2% final concentration. The samples were pre-equilibrated at 4° C. for an hour before each melt taking place at temperature increments of −1° C./min. The read-outs of Tm from each differential plot of fluorescence and temperature increment were used to plot a graph of Tm against concentration of additive used in the assay.

### Example 1—Inactivated Bacillus Spp Spores Administered Nasally are Capable of Protecting Against Influenza Virus Infection

The inventor hypothesized that bacterial spores that stimulate innate immunity may be utilised to provide rapid protection against respiratory virus colonisation.

### Results

Using a mouse model the inventors demonstrated that intra-nasal administration of killed spores (Spores^{K}) could prevent lethal challenge with virulent H5N2 (A/Aquatic Bird/Korea). FIG. 1 shows that two intra-nasal administrations of Spores^{K} provided 100% protection to 10 LD₅₀ lasting for at least 14 days post-challenge. This was equivalent protection to mice administered 2 intra-nasal doses of inactivated influenza H5N1 virions demonstrating protection levels equivalent to a vaccine formulation. In a similar study 60% protection was shown (FIG. 3). By titrating the intra-nasal dose of Spore^{K} the dose required to confer 100% protection was shown to be >5×10⁸ CFU (FIG. 4).

### DISCUSSION

This data shows that killed, and therefore metabolically inactive spores of Bacillus can confer protection to virus infections (influenza H5N2) when administered via a mucosal route, in this case nasal. The absence of influenza immunogens associated with the immunising dose suggests that protection is brought about by induction of innate immunity.

### Example 2—Identification of Sporulenes as an Adjuvant Molecule

The inventor hypothesised first that the ability to confer protective immunity in a host might result from one or more molecules contained within the spore. Further, that these molecules might confer adjuvant properties since adjuvants are known to carry inherent immunomodulatory properties.

### Results

In the first instance the inventor evaluated whether the spore carried adjuvant properties (FIG. 2) and then determined the nature of the spore-specific molecule that conferred immuno-modulatory properties (FIG. 5).

To demonstrate adjuvant properties inactivated H5N1 virions (A/Aquatic Bird/Korea) were used to dose mice by the intra-nasal route. The dose was defined by haemagglutinin (HA) units as 0.02 μg and 0.5 μg. As shown in FIG. 2 administration of 0.02 μg of H5N1 failed to confer protection to mice challenged with H5N2 (10 LD⁵⁰). On the other hand a dose of 0.5 μg (HA) of H5N1 virions provided 80% protection and, in this case, would result from the production of virus-specific neutralising antibodies which was independently confirmed. Strikingly, when either the low or high dose of H5N1 virions were mixed and co-administered to mice (2 nasal doses) 100% protection was observed. The most straightforward explanation is that spores, when mixed with so virions, boost their immunogenicity and thus act as a mucosal adjuvant. Similar findings were found with repeat studies using NIBRIG HA concentrations of 0.02 and 0.1 μg of HA (FIG. 7).

Spores carry unique terpenoid molecules that are found only in spores. These molecule referred are known as a sporulenes (Sporulene A, B and C). The cyclase enzyme (sporulenol cyclase) responsible for their synthesis is encoded by the sqhC gene. As terpenoids, sporulenes share similarities with known adjuvants such as squalene.

The inventor hypothesized that sporulenes produced by bacteria species, such as Bacillus and Clostridium, may play an integral role in the anti-viral properties that were observed and described in Example 1. To test this hypothesis, the inventor utilised a (sqhC) defective mutant strain of B. subtilis and tested it against its isogenic parent PY79 (SqhC+) as an adjuvant using H5N1 co-administration as a model.

Wild type spores and an isogenic mutant (sqhC) that lacks the sporulenol cyclase were prepared and then killed using autoclaving. Using 4 groups of mice a number of different dosing strategies were employed to immunise Spores^{K} and H5N1 (FIG. 5).

In the first instance the inventors dosed mice with either wt (Group 2) or sqhC (Group 1) spores by sub-cutaneous injection together with nasal administration of inactivated virons of H5N1 (A/Aquatic Bird/Korea). Using this strategy it was apparent that mucosal immune responses to H5N1 (secretory IgA) were significantly decreased when sqhC spores were used. In parallel the inventors administered animal groups using intra-nasal dosing with either wild type (Group 4) or sqhC (Group 3) killed spores that had been mixed with inactivated H5N1 virions. As shown in FIG. 5 H5N1-specific immune responses (Secretory IgA,) were significantly decreased when virions were co-administered with sqhC spores.

### DISCUSSION

Without wishing to be bound to any particular theory, the inventors believe that these data show that SqhC must play a role in enhancing local/mucosal immune responses to virus when adsorbed to spores. This is seen from the difference between groups 3 and 4 (FIG. 5).

Adsorption of H5N1 to spores is a key factor in enhancing local/mucosal immune responses since these are significantly greater than in mice dosed with spores and antigen by separate routes (Gps 1 and 2 vs Gps 3 and 4) or vs H5N1 delivery alone (FIG. 5).

Even when spores were delivered by a separate route (sub-cutaneous) they can still enhance mucosal (FIG. 5) immune responses (compare Gp 2 against HN1). This phenomenon is also found with alum.

In the above phenomenon, SqhC (and the products, Sporulenes, it generates) appears to also be important for enhancing mucosal immune responses when spores are delivered by a separate route, i.e., compare Gp 1 and Gp 2.

These experiments were performed using autoclaved (killed) spores (spore^{K}). The squalene cyclase gene, sqhC, encodes the squalene cyclase enzyme (a Sporulenol synthase) that bacterial spores utilise to produce sporulenes. Thus, without wishing to be bound to any particular theory, the inventor hypothesis that using live spores responses could be better if it ensures that SqhC or the Sporulenes under its control are not denatured. Alternatively, spores inactivated by another process, for example, UV—C or gamma irradiation may also ensure that SqhC is not deactivated, or its encoded Sporulenes are not denatured, and retain activity.

### Conclusions

Thus, in summary, the inventor has shown that adsorption to spores enhances mucosal immune responses to respiratory virus infection. Sporulenes (whose synthesis is dependent on SqhC), are tetracyclic isoprenoids, are key to this adjuvancy, and this could be specific to mucosal responses.

Genome searching has shown that a SqhC gene is present in Bacillus and Clostridia. Accordingly, the inventor believes that other Bacilli and Clostridia will also have SqhC homologues, orthologues or SqhC equivalents, and thus it would be expected that other spore-forming bacteria will also convey innate immunity protection against respiratory virus infection, based on the inventor's work.

### Example 3 —Bacillus Spp Produce Biosurfactants which Display Antiviral Properties

FIG. 6 shows a SEC (size exclusion chromatography) chromatogram of lipopeptides purified from Bacillus cells (in this case Bacillus spp. SG277). The principle lipopeptides identified were surfactin (peak Sec1), iturins (peak Sec2) and fengycins (peak Sec0). These lipopeptide biosurfactants are common to Bacillus species and found in varying levels between strains.

Using a purified SEC fraction (277-SEC, comprising peaks Sec0+Sec1+Sec2) we demonstrated that this Bacillus-produced material had strong denaturing activity against bovine serum albumin (BSA) (FIG. 8). Using a thermal shift assay we examined the ability of 277-SEC to denature BSA where the melting temperature of the protein is monitored vs increasing concentration of test product. Test products were 277-SEC, surfactin, and three bile acids (lithocholate, hyodeoxycholate, deoxycholate). Surfactin and the three bile acids were all obtained from commercial sources. The data shows that high dilutions of 277-SEC can denature BSA (indicated by the decrease in Tm). Surfactin, that is a component of 277-SEC, was also potent at denaturing BSA but less than 277-SEC but both 277-SEC and surfactin were far more potent than the other three biosurfactants.

### Conclusions

Bacillus-produced lipopeptides with biosurfactant activity have strong denaturing activity to proteins. The inventors believe that direct contact of Bacillus-produced biosurfactant lipopeptides can be used to rapidly denature viral envelope or capsid proteins.

### Example 4—Inactivated Bacillus Spp Spores Administered Nasally are Capable of Protecting Against SARS-CoV2 Infection

The general principles of the hACE animal model and its use for evaluating SARS-Cov2 infection are as described (Case et al. Cell Host & Microbe. 2020 28:1-10). hACE2 overexpressing (transgenic) mice are used for experimental in vivo studies. Mice are housed in biosafety class 3 facilities in groups. After acclimatization, anesthetised animals are dosed on day 0 and day 14 with killed Bacillus spores (Spores^{K}, 2×10⁹ per dose) by the intra-nasal route (20 μl per nostril). Spores are made in solution and killed by autoclaving (121° C., 15 psi, 30 min).

For challenge, 4-days after the last dose of spores (day 18) mice are anesthetised and administered ˜10⁵ plaque forming units (PFU) of SARS-Cov2 using the intra-nasal route. Body weight clinical signs and viral burden are monitored daily. For viral burden 4 tissues are examined 4 days after challenge (FIG. 9), these being lungs, spleen, heart and nasopharanyx are examined. For nasal samples, washes are used to retrieve and extrapolate viral PFU. For heart, lung and spleens, burden is extrapolated by qPCR.

### Discussion

The data shows that counts of SARS-Cov-2 present in the lungs, spleen, heart and nasal washes are significantly reduced compared to control groups where viral burden remained high indicative of infection. Since spores lack any SARS-Cov2 immunogen, and without wishing to be bound to any particular theory, the inventors reason that the effect on infection is most likely caused by innate immunity and resembles that of influenza prevention (Example 1). The inventors further reason that the underlying mechanism must be similar and derive from interaction of spores with TLRs possibly involving SqhC.

### Example 5—Bacillus Spp Spores Demonstrate an Adjuvant Effect Against the SARS-CoV-2 Spike Protein

Mice (Balb-C; female, 8-weeks old) were immunised on day 1 with recombinant SARS-CoV-2 Spike protein (Sino-Biological (Cat: 40589-V08B1; amino acids 16-1213)). Protein was mixed with adjuvant (Addavax, Invitrogen) and administered by intramuscular injection. On day 14 and day 28 mice were boosted nasally (intra-nasal, i.n.; 20 microlitres) with either PBS buffer (Control) or purified spores of B. subtilis strain PY79 (2×10⁹ CFU/dose suspended in PBS buffer).

### Results

Antigen (Spike)-specific antibody responses were determined on day 42 in serum (IgG) (Panel A), saliva (SIgA) (Panel B) and lungs (secretory IgA, SIgA) by ELISA (Panel C).

As illustrated in FIG. 10, boosting mice with purified spores of B. subtilis strain PY79 using a nasal route, augments immunity by increasing the titre of antigen-specific SIgA in the lungs and saliva, as well as IgG in serum.

### Conclusions

The ability to augment mucosal responses such as SIgA, is important for the existing coronavirus vaccines and demonstrates that spores exert a unique and novel adjuvant effect on an antigen administered by a parenteral route. As such, this data demonstrates that spores have utility for improving existing coronavirus vaccines by enhancing their performance and potentiating the immune response.

### Example 6—Bacillus Spp Spores Provide Protection Against Infection with SARS-CoV-2

Male K18-hACE2 transgenic mice (n=5/group) were treated with three weekly intra-nasal doses (day 1, 7 and 14) of heat inactivated B. subtilis spores (SPOR-COV) at 1.5×10⁹ CFU/dose (30 μl/dose). Spores of B. subtilis strain SG188 (SPOR-COV) were autoclaved (121° C., 15 psi, 20 minutes) and had no viability. Seven days after the last dose, mice were challenged (intra-nasal) with SARS-CoV-2 (5×10³ PFU/50 μl/dose).

### Results

FIG. 11 illustrates the percent survival (Panel A), body weight (Panel B) and disease score (Panel C) of mice, following a treatment regimen of (i) B. subtilis spore and mock infection, (ii) B. subtilis spore and SARS-CoV-2, or (iii) SARS-CoV-2 and PBS. As shown in Panel A, the inventors surprisingly found that nasal dosing of mice with inactivated B. subtilis spores provided 80% protection to a lethal dose of SARS-CoV-2. In contrast, treatment with SARS-CoV-2 and PBS resulted in a 0% survival rate in mice.

### Conclusions

This data shows that heat inactivated spores of B. subtilis can confer effective protection to coronavirus infections when administered via a mucosal route, as demonstrated by 80% survival of mice treated with B. subtilis spores. Accordingly, this demonstrates that spores have the ability to protect against SARS-CoV-2 and improve survival rates following coronavirus infections.

### Example 7—Spore Pre-Treatment Recruits CD4⁺ and γδ T Cells

Mice were treated with three weekly intranasal doses of 1.5×10⁹ CFU/30 μl of heat-killed spores (SPOR-COV). Seven days post the last dose of spores, mediastinal LN (mLN) (FIG. 12A) and bronchoalveolar lavage fluid (BALF) (FIG. 12B) were collected for T cell subset classification.

### Results

As illustrated in FIGS. 12A and 12B, spores induced an increase in T cell recruitment into the lung draining lymph node (LN) and the lung alveolar space.

Mice (male C57BL/6, 5 mice per group) were treated with three weekly intranasal doses of 1.5×10⁹ CFU/30 μl of heat-killed spores and challenged with 1×10² PFU/50 μl of H1N1-PR8 virus (influenza) seven days post the last dose of spores. Mice were culled at five days post infection to collect lungs for virus titre detection and BALF for immune cell analysis.

Results As shown in FIG. 13A, pre-treatment with spores reduced virus load in the lung following H1N1 infection. Additionally, FIGS. 13B and 13C illustrate that spore pre-treatment enhanced CD4⁺, CD8⁺ and γδ T cell recruitment into lung alveolar space during H1N1 infection. Furthermore, the inventors surprisingly found that spore pre-treatment reduced NK cell recruitment into lung alveolar space at five days post H1N1 infection.

### Conclusions

Thus, the inventors hypothesized that bronchoalveolar lavage fluid CD4⁺ and γδ T cells recruited by spore treatment, may have a regulatory role that ameliorates tissue damage during H1N1 infection. Additionally, due to the reduced levels of NK cells observed in the lung alveolar space during H1N1 infection, the inventors also hypothesized that exuberant NK cell infiltration may contribute to lung tissue damage in viral pneumonia.

### REFERENCES

1. Frieman M, Heise M, Baric R. SARS coronavirus and innate immunity. Virus Res. 2008; 133(1):101-12; doi: 10.1016/j.virusres.2007.03.015.
2. Iwasaki A, Pillai P S. Innate immunity to influenza virus infection. Nat Rev Immunol. 2014; 14(5):315-28; doi: 10.1038/nri3665.
3. Van Hoeven N, Fox C B, Granger B, Evers T, Joshi S W, Nana G I, et al. A Formulated TLR7/8 Agonist is a Flexible, Highly Potent and Effective Adjuvant for Pandemic Influenza Vaccines. Sci Rep. 2017; 7:46426; doi: 10.1038/srep46426.
4. Hong H A, Duc le H, Cutting S M. The use of bacterial spore formers as probiotics. FEMS Microbiol Rev. 2005; 29(4):813-35; doi: S0168-6445(04)00089-0 [pii]10.1016/j.femsre.2004.12.001.
5. Song M, Hong H A, Huang J M, Colenutt C, Khang D D, Nguyen T V, et al. Killed Bacillus subtilis spores as a mucosal adjuvant for an H5N1 vaccine. Vaccine. 2012; 30(22):3266-77; doi: 10.1016/j.vaccine.2012.03.016.
6. Barnes A G, Cerovic V, Hobson P S, Klavinskis L S. Bacillus subtilis spores: a novel microparticle adjuvant which can instruct a balanced Th1 and Th2 immune response to specific antigen. Eur J Immunol. 2007; 37(6):1538-47; doi: 10.1002/eji.200636875.
7. de Souza R D, Batista M T, Luiz W B, Cavalcante R C, Amorim J H, Bizerra R S, et al. Bacillus subtilis spores as vaccine adjuvants: further insights into the mechanisms of action. PLoS One. 2014; 9(1):e87454; doi: 10.1371/journal.pone.0087454.
8. Czerkinsky C, Cuburu N, Kweon M N, Anjuere F, Holmgren J. Sublingual vaccination. Hum Vaccin. 2011; 7(1):110-4.
9. Song J H, Nguyen H H, Cuburu N, Horimoto T, Ko S Y, Park S H, et al. Sublingual vaccination with influenza virus protects mice against lethal viral infection. Proceedings of the National Academy of Sciences of the United States of America. 2008; 105(5):1644-9; doi: 10.1073/pnas.0708684105.
10. Wang X, Hu W, Zhu L, Yang Q. Bacillus subtilis and surfactin inhibit the transmissible gastroenteritis virus from entering the intestinal epithelial cells. Biosci Rep. 2017; 37(2); doi: 10.1042/BSR20170082.
11. Kracht M, Rokos H, Ozel M, Kowall M, Pauli G, Vater J. Antiviral and hemolytic activities of surfactin isoforms and their methyl ester derivatives. J Antibiot (Tokyo). 1999; 52(7):613-9; doi: 10.7164/antibiotics.52.613.
12. Smith M L, Gandolfi S, Coshall P M, Rahman P. Biosurfactants: A
13. Y. Sun and C. B. Lopez (2017) Vaccine 35:481-488 The innate immune response to RSV: Advances in our understanding of critical viral and host factors
14. H. Ganjian, Rajput, C., Elzoheiry, M. and Sajjan, U. 2020 Frontiers in Cellular and Infection Microbiology https://doi.org/10.3389/fcimb.2020.00277 Rhinovirus and Innate Immune Function of Airway Epithelium
15. Gao, Z., S. Wang, G. Qi, H. Pan, L. Zhang, X. Zhou, J. Liu, X. Zhao and J. Wu (2012). “A surfactin cyclopeptide of WH1fungin used as a novel adjuvant for intramuscular and subcutaneous immunization in mice.” Peptides 38(1): 163-171.
16. Gao, Z., X. Zhao, S. Lee, J. Li, H. Liao, X. Zhou, J. Wu and G. Qi (2013). “WH1fungin a surfactin cyclic lipopeptide is a novel oral immunoadjuvant.” Vaccine 31(26): 2796-2803.
17. Mittenbuhler, K., M. Loleit, W. Baier, B. Fischer, E. Sedelmeier, G. Jung, G. Winkelmann, C. Jacobi, J. Weckesser, M. H. Erhard, A. Hofmann, W. Bessler and P. Hoffmann (1997). “Drug specific antibodies: T-cell epitope-lipopeptide conjugates are potent adjuvants for small antigens in vivo and in vitro.” Int J Immunopharmacol 19(5): 277-287.
18. Yoshino, N., R. Takeshita, H. Kawamura, K. Murakami, Y. Sasaki, I. Sugiyama, Y. Sadzuka, M. Kagabu, T. Sugiyama, Y. Muraki and S. Sato (2018). “Critical micelle concentration and particle size determine adjuvanticity of cyclic lipopeptides.” Scand J Immunol: e12698.

1.-32. (canceled)

33. A method of (i) treating, preventing or ameliorating a respiratory virus infection, and/or (ii) stimulating innate immunity in immune-prophylaxis against a respiratory virus infection, the method comprising nasally administering a therapeutically effective amount of a live or dead bacterial spore to a subject in need of such treatment, wherein the live or dead bacterial spore is a Bacillus spp spore.

34. The method of claim 33, wherein the live or dead bacterial spore is an immune stimulant, an innate immunity stimulant or a prophylactic immune modulator.

35. A method according to claim 33, wherein the live or dead bacterial spore is adapted to exert an adjuvant effect on an antigen administered parenterally.

36. A method according to claim 35, wherein the antigen is a coronavirus vaccine.

37. A method according to claim 33, wherein the live or dead bacterial spore is used as a boost to augment immunity by increasing the titre of antigen-specific secretory IgA (SIgA) in the lungs and saliva, and optionally of IgG in serum.

38. A method according to claim 33, wherein the live or dead bacterial spore augments or stimulates a mucosal response.

39. A method according to claim 33, wherein the live or dead bacterial spore augments or stimulates production of secretory IgA (SIgA).

40. A method according to claim 33, wherein the live or dead bacterial spore increases recruitment of CD4⁺, CD8⁺ and/or γδ T cells to the site of viral infection.

41. A method according to claim 33, wherein the live or dead bacterial spore reduces natural killer (NK) cell recruitment into the lung following the virus infection.

42. The method according to claim 33, wherein the respiratory virus is selected from a group consisting of: Respiratory syncytial virus (RSV), Coronavirus and Rhinovirus.

43. The method according to claim 33, wherein the respiratory virus is a Coronavirus, preferably SARS-CoV-2.

44. The method according to claim 33, wherein the live or dead bacterial spore is applied mucosally to the patient.

45. The method according to claim 33, wherein the bacterium comprises a squalene cyclase gene (sqhC), or a homologue, orthologue or equivalent thereof, and/or wherein the bacterium comprises one or more sporulene, optionally wherein the bacterium comprises a nucleic acid sequence comprising the nucleic acid substantially as set out in SEQ ID No: 28, or a fragment or variant thereof having at least 70% sequence identity to SEQ ID No: 28.

46. The method according to claim 33, wherein the live or dead bacterial spore produces or comprises a sporulene family member, optionally wherein the sporulene family member is Sporulene A, B and/or C.

47. The method according to claim 33, wherein the live or dead bacterial spore:
(i) produces or comprises one or more non-ribosomal peptides, optionally wherein the non-ribosomal peptide is a lipopeptide selected from a group consisting of: members of the Fengycin family, members of the Surfactin family, and members of the Iturin family;
(ii) further produces or comprises a glycolipid, preferably wherein the glycolipid is a Rhamnolipid or an active derivative thereof, and/or a Sophorolipid or an active derivative thereof; and/or
(iii) further produces or comprises a lipopeptide selected from a group consisting of: Mycosubtilin; Mojavensin A; and Kurstakin, or an active derivative of any of these lipopeptides.

48. The method according to claim 33, wherein the bacterium comprises one or more of the nucleotide sequences selected from the group consisting of: SEQ ID No: 1 to 6, 28 and 29, or variants or fragments thereof having at least 70% sequence identity to SEQ ID Nos: 1 to 6, 28 and 29, optionally wherein the bacterial spore is dead, and wherein the dead bacterial spore is applied nasally.

49. A method of treating, preventing or ameliorating a virus infection, the method comprising administering, or having administered, to a patient in need of such treatment, a therapeutically effective amount of an antiviral and/or innate immune stimulation composition comprising at least one sporulene family member and/or a lipopeptide selected from the group consisting of: a member of the Surfactin family, a member of the Iturin family, and a member of the Fengycin family, or an active derivative of any of these lipopeptides.

50. The method according to claim 49, wherein:
(i) the composition further comprises a glycolipid, preferably wherein the glycolipid is a Rhamnolipid or an active derivative thereof, and/or a Sophorolipid or an active derivative thereof;
(ii) the composition further comprises a lipopeptide selected from a group consisting of: Mycosubtilin; Mojavensin A; and Kurstakin, or an active derivative of any of these lipopeptides; and/or
(iii) the virus is selected from the group consisting of: Respiratory syncytial virus (RSV), Coronavirus and Rhinovirus.

51. A foodstuff or dietary supplement comprising a live or dead Bacillus spp. spore and optionally one or more food grade ingredients.

52. A pharmaceutical composition comprising a live or dead Bacillus spp. spore, and a pharmaceutically acceptable vehicle or carrier.


SEQUENCE LISTING (29 sequences)

SEQ ID NO: 1 - DNA, 1555 bp, Bacillus amyloliquefaciens
tttatcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacatgaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctcctttctaa

SEQ ID NO: 2 - DNA, 1555 bp, Bacillus amyloliquefaciens
tttatcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactacgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctcctttctaa

SEQ ID NO: 3 - DNA, 1555 bp, Bacillus amyloliquefaciens
tttatcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctcctttctaa

SEQ ID NO: 4 - DNA, 1273 bp, Bacillus subtilis
acgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctcctttctaagga

SEQ ID NO: 5 - DNA, 1555 bp, Bacillus amyloliquefaciens; identical to SEQ ID NO: 3

SEQ ID NO: 6 - DNA, 1555 bp, Bacillus amyloliquefaciens; identical to SEQ ID NO: 3

SEQ ID NO: 7 - DNA, 1203 bp, Bacillus amyloliquefaciens
atgaacaatcttgcctttttatttcctggacaagggtctcaatttgtaggaatgggcaaacaattttggaatgattttgtgctcgcaaagagattgtttgaagaagcgagcgatgcgatctccttggatgtaaaaaaactgtgttttaacggagatatgaatgaattgacaaagacaatgaacgcgcagcccgctattttaacggtcagtgttattgcttttcaagtgtatatgcaggaaataggggtgaagccccgcttcctggcaggccatagcttaggcgaatattcagcgcttgtctgtgccggcgccctttcttttcaggatgccgttacacttgtaaggcagcggggaattcttatgcagaatgcggatcctcagcagcaggggacgatggccgccgtgactcacctctctcttcaaacgttgcaggaaatatgttcgaaagtgtcgacggaagactttccggcaggagtagcctgcatgaattcagaacagcagcatgtgatttccggacaccggcaagctgtggaacgtgtcatcaagatggcggaggaaaagggagcggcatacacttatttgaatgtcagtgcgccttttcacagttcgctgatacgatcagcatctgaacaattccagactgtattacaccggtattccttccgggatgccgcatggccgatcatttcaaatgtcaccgcacgcccttacagcagcggaaattcaatcagcgaacatctcgagcagcacatgacgatgccggtaagatggacggaatcgatgcattacttgcttttacacggagtcacagaagtcatcgaaatgggtccgaacaatgtcttagccggtctgctgagaaaaacaacgaatcacattgtaccttatcccttaggacagacatctgatgttcacttgctttccaattcagcagaaagaaagaaacatattgtccgtttacgcaaaaaacaactgaataaattgatgattcaatccgtcattgcgcgaaattacaacaaggattcagcggcttattccaatatgacgacggcattatttacgcaaatccaagagctgaaagagagaatggaaagacatgaaaatgagctctcagaacaagagctcgaacattcgatccatttatgcaaattaatttgcgaggctaaacagcttccggattgggaagaattgcggattttaaaataa

SEQ ID NO: 8 - DNA, 11949 bp, Bacillus amyloliquefaciens
atgtataccagtcaattccaaaccttagtagatgtcattcgggaaagaagcaatatctctgaccgcgggatccgttttatcgaatccgataaaaacgagacggttgtctcttatcgccaattgtttgaagaggcgcaagggtatcttggctatttacagcatatcggcattcagccgaagcaggaaattgtatttcaaatccaagaaaacaaatcatttgtcgttgctttttgggcttgtatattaggaggaatgatcccggtgccggtcagtatcggagaagatgatgaccataagctgaaggtctggcgcatttggaatatattaaatcacccgtttctgattgcctctgaaaaagtattggacaaaataaagaaatacgctgcagaacacgatttacaggatttccatcatcaattaaacgaaaaatctgacatcattcaagatcaaacctacgattaccccgcttcgttttatgaacctgatgcggatgaactcgcctttatccaattttcttcaggatcgacaggagatccaaaaggagtcatgttaacgcatcacaacttaatacataacacgtgcgccattgggaatgccctagccgttcattcgagagactctttcttatcatggatgcctttaacgcatgatatggggctcatcgcctgccaccttgttcccttcataaccggaatcaatcaaaatctgatgcctacagaattatttattcgcagacctattctttggatgaaaaaagctcatgaacataaagccagtattctatcctctcctaatttcggatacaactacttccttaaatttctgaaaaacgaaccagactgggatttatcccacatcaaggtcatcgcaaacggtgcagaaccgatattgccggagctctgtgacgaatttttgaaaagatgcgcagcattcaatctgaaaagatccgccattttgaatgtttacggtttagcggaagcttcggtcggcgcagcattctctaaattaggtaaagaattcgttcccgtttatttgcatcgcgatcatttaaatctcggtgaaagagctgtaaacgtcagcaaagaggatcaaaattgcgcttcattcgtcgaagtgggacgacctattgactattgtcagcttcggatctccgatgaagcaaatgaaagagtagaagacggaatcatcggccatatccagatcaaaggagacaatgtgactcaagggtattataacaaccccgagagtacggaaaaagcgctgacttctgacggctgggtaaaaacgggagacctcggattcattagtgaaagtggtaacttagtcgtaaccggaagagaaaaggacattattttcgtgaacggaaaaaatatctacccgcacgatattgaacgggtagcgattgaaatggaagaggttgacttaggaagggttgccgcctgcggtgtatatgatcaaaagacacaaagcggagaaatcgtgctctttgttgtttacaaaaaatcacctgaaaaattcgcaccgcttgtcaaagagataaaaaagcatttgctcaagcggggcggctggagcataaaagatgtccttccgatccgaaaactccctaaaacaaccagcggaaaggttaaacgctacgaacttgccagacagtatgaggcagggaatttttcaacagagtctgccgccatcaatgaatatttggagagcagcccggaaacgtccggacagactcccattcatgaaattgaaacggaattactgtctatcttttccgatgtgctcaatgggaaaaaggttcacctcgctgacagttattttgatatgggagcaaattcattacagttatcgcagattgccgagcgcatagaacagaaattcggacgcgagcttgccgtttcagatctctttacgtatccttctatcactgatttagcggcgtatctgtctgaaagccgggctgaaatcaagcaggacgtggcagctaaaccaagccatgtgacaccgaaagatatcgccattatcgggatgtcgctcaatgtccctggagcatcaactaaaaatgatttttggaatctgcttgaaaaaggtgagcacagcattcgagaataccctgcatcccggctgaaagatgcggcggattatttaaagtccatccaaagcgaaatcaatgagaatcagtttgtgaagggcggctatttagatgaaatcgaccgctttgatttctcgttcttcggtttagctcctaaaacggctcagtttatggaccctaaccaaagactgtttttgcagtctgcatggcatgcgattgaagatgcgggctatgccggcggcagcatgaacgggagccgtgtcggggtatatgcagggtactcgaaggtgggctacgattatgaacgtctcctttctgcgaattatccggaggagcttcatcaatatatcgtgggcaatctcccttccgtgttagccagccgaatcgcttatttcttaaatttaaaagggccggcggtcacagtcgatacggcgtgctcctcatcgcttgccgccgttcatatggcatgtaaatctttaatatccggcgattgtgaaatggctcttgccggcggtatccggacatcgctcttgccgatctgtatcggacttgatatggaatcttcggacgggtacacgaaaacgttcagcaaagattcagacggtactggcacaggtgaaggcgcggccgcagtcctgctgaaacctctgcaggatgctgttcgcgacggagaccatatttacggcgtaatcaagggaagcgcgttgaatcaagacggaacaaccgccgggattacagcaccgaatccggcagctcagactgaggtcattgagacggcctggaaagacgcgggcattgcccctgaaacactgtctttcatcgaagcgcatggcaccggaacgaagctcggcgatccggttgaatttaacgggctttgtaaagcgtttgaaaagtatacggcaaaaaaacaattttgtgcgattggttctgttaaatcgaacatcggtcatttgtttgaagcggcaggcatcgtcgggctgatcaaatctgtcctcatgctgaatcacaagaaaaatccgccgttagtgcactttaatgaacctaatccgctcattcattttcactcttcaccattttacgtaaaccaggaagctgcagcgttcccatccggtgatgagccgctgcgaggcggagtcagctcatttggctttagcggaacgaacgctcatgtggtattggaagaatatatttctcaaagtgagtatgcgcccgaggatgaacatgggccgcacctatttgttttatccgctcatactgaaaaatcactctatgaactcgcacagcagtaccggcaatatgtatcggatgacagccaagcttcattaaagtccatttgctatacagccagtacgggcagggctcatttggatcatggcattgccatgattgtatccggtaaacaagaactatcggataagctgacccgcctgattcagggagacagaaaccttcccggtgtatacatcggctacaagaatatgaaggaaatgctgcccgctcataaagaagagctgaataaacaagcagccgcactgattaagcagcgtttacgtacgcaagatgaacggatcacatggctgcatcgcgccgccgaattatttgtgcaaggagccgttatcgattggcgcgcgctttattcaggtgaaactgtacaaaagacgccattgcccttgtatccgtttgaacggagccgatgctgggctgaagctgaccaattgcgcttaaacgaggacgaaaagagaggagaagcggcattgaatatcaatcaatcgaagtcgcatattgaatccttcctgaaaactgtaatcagcaatacttcggggatcagagcggaggaactcgatctgaatgctcattttatcggactcggaatggattctatcatgctgtcacaggtcaaaaaagccatcgcggacgaatttggggcagacatcccgatggatcgtttttttgatacgatgaacaaccttcaaagtgtcatagattacttggctgagaccgttccaacgtcctttgcatccgctccgcctcaagagaatgttccggcgcaggaaatgcaggtcatttcagaagcacagtctgaatcggatcgcagagaaggtcatcaagagcatatgctcgaaaaaataatcgcttctcagaatcaattaattcaggataccttgcaagctcaattaaatagctttaatttgttgagaaacagcggacatcattccgatgagaaagaatacgctaaagcgcaagagagatcaattccttctgtccagcaggggcctccggccgtcactgcagaaaagaaagcggctcaagaagcgaaaccctatgttcctttccagcctcagaacctgcatgaacagggacactataccgcacggcaaaaacaatacttagaagatttcatcaagaaatacgcagataaaacgaaaggttccaaacaatatacggacaacacccgatttgctcatgcaaacaaccgcaacttgtccagcttccgttcatattggaaggaaatcgtatacccgattatcgccgaacgttctgacggttctaaaatgtgggatattgacggaaatgaatatatcgatgtcaccatgggattcggggttaaccttttcgggcatcatccttcctttattacacaggttatcgatgattcagcccgttcttcattgcctccgctcggaccgatgtcagatgtcgccggtgaagttgccgaccggatccgcacatgtaccggggtagaaagggtcgctttctataattccggaacagaggccgtcatggttgccctgcgtttggcgcgggcggcaacaggaagaaagaaagtggtggcgttctcgggctcttatcacggcacgtttgacggcgtattaggggttgccggcacaaaaggcggagctgcgtctgcgaatccgctggctcctggtatactgcagagctttatggatgatttgattattttacattacaacaatcccgattctctggacgtgatccgcagtcttggtgatgaattggcagccgtactggtggaaccggtacaaagccgcagaccggatttgcagccgcgggcatttttgaaagaattgcgggcgatcacgcagcaatccggaacagctctgattatggatgaaattattaccggatttcggatcggtctcggcggcgcacaggaatggttcggcattcaggctgatttagtgacctacggaaaaatcatcggcggcggacagccgttaggggtagttgccggaaaagctgagttcatgaatgcgatcgacgggggtacctggcagtatggggacgattcctacccgcaagacgaggcgaaacgcacgtttgtggccggaaccttcaatactcatccgcttaccatgagaatgtcattagccgtgcttcgtcatttacaaaccgagggagaacatctgtatgagcagcttaatcaaaaaacagcctacttggtggatgagctgaatcgctgcttcgaacaagcgcaagtgcctatccgcatggttcgattcggttctttattccggtttgtctcatcgcttgataatgacttgttcttttaccatctcaactataaaggtgtctatgtgtgggaaggacgcaactgcttcttgtctgcggcgcataccgctgatgatatcgaaaagattattcaagcggtgaaagacacggtggaggatcttcgccgaggcggatttattccggaaggcccggactcccctgatggcggaggccgtaaaaagtccgggacgcgcgagctttcacctgaacaaaagcagttggttatggcatcccattacgggaatgaagcgtccgccgctttaaaccagtccattatgctgaaagtggagggcgaactgcagcatacaccattaaaacaagccgtccggcatatcgttggccgtcatgaagctttacgtacggtgattcatcccgatgacgaggtacagcaagtgcaggaacggatgaatatagaaataccagtcattgattttaccgttcacccgcatgaacatcgggagtcggaaattcaaaaatggctgacagaagatgccaagcggccgttccatttccatgaacaaaagcctttgtttagaatccatgtgcttacatcggctcacaatgaacatctgattgtgctcacgttccatcatatcattgccgatggatggtcaatcgccgtatttgttcaagaactggagagcaactacgcggcaatcgtacaaggaaaaccgatttcaccgaaagaggcagatgtttcgtttcgccaatacttagactggcagcaggcacagattgacagcggccattatgaagaaggggtccgttattggcggcgtcatttctctgaaccgattcagcagccaattctgccgagcacaggttctgtccgttatccgaacgggtatgagggagaccggtgtaccgtcaggcttggacggccattgagcgaggctttaaggtcattaagcattcagatgaaaaatagcgtatttgtgacaatgctgggtgcatttcatctttttctgcaccggcttaccaaacagtcaggccttgtgatcgggatccccgcagcaggtcaatcgcatataaaacagcatgatctgattggaaattgcgtcaatatgattccggtgaagaacacgtctacttcagaaagcactttaaccggttatcttggcagtatgaaagaaagcgtgaatcttgcaatgcggcaccaagccgtcccgatgacactggtggccagagagcttccgcacgatcaagtgccggatatgcgtattatctttaatttagacaggccttttcgaaagctgcatttcggaaaggcggaagcggagcccgttgcatacccggtaaaatgcaccctgtacgatttatttcttaacataacagacgcgcatcaagaatatgttcttgatttcgactttaatacgaacgtcatcagtccggaaatcatgaaaaagtggggagcgggttttacaaatttgctgcaaaaaatggttgagggggattcaatccctcttgacgccatgatgatgttttccgatgaagaacagcatgatttacaaaaactgtatgccgaacaccagaagcgggtctcttcaataggaagcaatacagcaaatttcactgaagcctacgaggcgccgataaatgaaacggaacggcagctggcgcggatttgggaggaacttttcggccttgaacgggtcggcagatcagatcgctttctggctctgggaggaaactcgctccaggcgacgcttatgctttccaaaattcagaagacatttcatcaaaaggtttccatcggacaatttttcaatcaccagactgttaaggaattagcacatttcattcagaacgaaacaaaagtcgtgcacctcccgatgaaagctgccgagaaaaaagcgtattacccgacatcgccggcgcagcaaagagtatatttcctgcaccaactggaaccggatcagctggcgcaaaatatgttcggccaaatatcaataacagggaagtacgatgagcaagccctgatctcatctcttcaacaagtgatgcagcggcacgaagcgtttcgcacgtatttcgacattatagatggcgatatcgttcagaaacttgaaaacgaagttgattttaacgttcatgtccggacaatgagccgggacgaatttgatgcctattcagaccggtttgtaaaaccgttccgcctggaccaagctccgttagttcgtgcggagctgatcaagattgaaaacgagcaggccgaactgctcatcgatatgcatcatatcatttcggatggttattccgtcaacatccttacaaatgaattgctggctttatatcatcagaaaccattaccggacattgaatttgaatataaagatttcgcagaatggcaaaaccaacggctgaatgaggatgccatgaagcggcaggagacatattggctggaacaatttcaagacgaaattcccatccttgacctgccgacagacggttcaaaagcggcagaacggtcttctgagggacagcgtgtgacatgctccttacagccggatgtcatccgttcgcttcaagatttggcgcaaaaggcggaaaccactctctatacggtgcttctggccgcctataatgtgctgcttcataaatataccggacaagaagacattgtcgtaggcacgcctgcttcaggaagaaatcatccggatatcgaaaaaatcatcggtattttcatacaaaccatcggaatccggacgaagccgcacgccaatagaacgtttacggattatctggaagaagtaaagcggcagacgcttgacgctttcgaaaaccaagactatccattcgaccggcttgtggagaaattaaatgtgcagcgggaaacaaccggaaagtctctgtttaacacgatgtttgtgtttcaaaacattgaatttcatgaaatccggcacaatgaatgtacatttaaagtgaaagaacgaaatccaggggtctctttgtatgatttgatgctcacgatcgaagatgccggtcaacagatagagatgcattttgattataaaccgggacgattcacaaaagacaccattgaacagatcaccagacactataccggcattttaaacagtcttgttgagcagccggagatgacattgtcttccgttcctatgctgtctgaaaccgaacggcatcaactgctgacggagtgtaacggcacaaagacgccgtatccgcataacgaaacagtaacccgatggtttgaaatgcaggcggaacagagtcccgatcatgcagccgttatttttggcaatgagcggtatacgtacagacagctcaatgaacgggcgaaccgattggcgcggacgttacggacaaaaggcgtacaagcggatcaattcgttgccatcatctctccgcatcgcatcgagttgattgttggtattttggctgttctgaaatcaggcggcgcatacgtgcctattgatcctgaatatccggaagatcggatccaatatatgctgagagattcaagggcggaggttgtgttgacacagcgcagcctgctggatcaattaccgtatgatggtgacgttgtgcttttggatgaggaaaactcataccatgaggatcactcgaatcttgaatcggacagcgatgcgcatgatttggcctacatgatctatacgtcaggttccacgggaaatccaaaaggtgtcctcattgagcatcagggactggctgattatatttggtgggcgaaagaggtttatgtaagaggtgagaaaaccaacttcccattatactcttccatctctttcgatctgactgtgacctcgatatttacaccgctggttacgggaaataccatcattgtctttgacggcgaagacaaaagcgctgtgctttctgagattatgcgggactcaagaatagacatgatcaaattgaccccggcacatctgcacgtcatcaaggagatgaatatcggtggcggcaccgcaatacggaaaatgattgtcggcggagaaaatttaagcacccgtctggccaaaagtgtcagcgagcagtttaaaggccggctggacattttcaatgaatacggaccgacggaagctgtcgtcggatgtatgatttatcacttcgacgcagaacgggacaagcgggaatttgtaccgatcggcactccggctgccaacacggatatttatgtggccgatgcaagcagaaatctggttccgatcggggtaatcggcgaaatatatatcagcggaccgggtgttgccagagggtattggaaccgtccggatttaacggcagagaaatttgttgaaaacccgtatgtcccgggagcgaagatgtacaaatcaggggatttggctaagcggttgaaggacggaaaccttgtatatatcgggcgcgttgatgaacaagtcaaaatcaggggataccgaatcgagcttggtgaaattgaagcagcaatgcataacgcggaagcggtgcaaaaagccgcggttacagtgaaagaagaagaagacggcttaaaacaattatgcgcgtattacgtaagcgacaagcctatagcggctgcgcagcttagggaacaattgtcatcggagcttccggactacatggttccgtcctattttgtccaactggagcatatgccgttaacgtccaacgggaaaataaaccgtaaggcactgccagcaccagaagcgagtctgcagcagacagctgaatatgttccgccgggtaatgagacggagtccaaactgacagatttatggaaggaagtgctcggaataagccatgcggggatcaaacataatttctttgatctcggaggcaactccatccgagcggctgccttggccgccagaattcacaaagagctggatgtgaatctgtctctcaaagacatattcaagtttcctaccattgaacaattggctgacaaggcgttacacatggacaaaaaccgatatgtaccgattccggctgcaaaggaaatgccatattatccggtttcttcagctcagaggcgtatgtatttgttaagtcacacagaaggcggcgagctgacttacaatatgacgggtgccatgaatgtggaagggacgatcgatcccgaacggttaaacgccgctttccgaaaattaatcgcgcgtcatgaagcgttgcggaccagctttgatttatatgaaggcgagccggcacagcgtattcatcagaacgtcgactttacgatagaacggattcaagcaagcgaagaagaagcggaagaccgtgtgcttgatttcatcaaagcgtttgacttagccaaaccgccgctgatgcgggccggactgattgaaattgaacctgcgcggcacgtgcttgtggttgatatgcatcatatcatttctgacggcgtgtccgtcaatattctgatgaaagatttaagccgaatctacgaggggaacgaaccggacccgctctctattcaatataaagactttgcagtttggcagcaatcggacattcagaaacggaacatcaagagccaggaagcgtattggctggatcagtttcacagtgatattcctgtactggatatgcctgcggattatgagagacctgccatacgcgattacgaaggcgaatcatttgaatttcttatacccgaacacttgaaacagcgtttaagccaaatggaagaagacacaggagcaacactgtatatgattttattggcggcctatacgattcttttatccaggtacagcggacaagaagatattatcgtaggaacgccatctgccgggcggactcatttggatgtagagccggtcgtgggaatgttcgtcaatacgttagtcattcgcaatcacccggcgggccgtaaaacatttgatgcctacttaaacgaagtaaaagaaaacatgctgaacgcctataaaaatcaagactatccattggaagaattaattcagcatctgcatcttccaaaagattcaagccgcaatcctttattcgatacgatgtttgtgctgcaaaatctcgatcatgctgaattgactttcgattctcttcaactcaagccgtattcatttcatcatccggttgccaaattcgatttgaccttgtcgattcaggcggaccaagacaactatcacggactgtttgaatattcgaaaaaactgtttaagaaaagcagaatcgaggttttatcaaacgactacttacacattctatcggcgattttggaacaaccaagcattctaattgaacatatcggattgagcggcagcaatgaggaagaagagaacgcgcttgattctattcaattgaacttttag

SEQ ID NO: 9 - protein, 400 aa, Bacillus amyloliquefaciens
MNNLAFLFPGQGSQFVGMGKQFWNDFVLAKRLFEEASDAISLDVKKLCFNGDMNELTKTMNAQPAILTVSVIAFQVYMQEIGVKPRFLAGHSLGEYSALVCAGALSFQDAVTLVRQRGILMQNADPQQQGTMAAVTHLSLQTLQEICSKVSTEDFPAGVACMNSEQQHVISGHRQAVERVIKMAEEKGAAYTYLNVSAPFHSSLIRSASEQFQTVLHRYSFRDAAWPIISNVTARPYSSGNSISEHLEQHMTMPVRWTESMHYLLLHGVTEVIEMGPNNVLAGLLRKTTNHIVPYPLGQTSDVHLLSNSAERKKHIVRLRKKQLNKLMIQSVIARNYNKDSAAYSNMTTALFTQIQELKERMERHENELSEQELEHSIHLCKLICEAKQLPDWEELRILK

SEQ ID NO: 10 - protein, 3982 aa, Bacillus amyloliquefaciens
MYTSQFQTLVDVIRERSNISDRGIRFIESDKNETVVSYRQLFEEAQGYLGYLQHIGIQPKQEIVFQIQENKSFVVAFWACILGGMIPVPVSIGEDDDHKLKVWRIWNILNHPFLIASEKVLDKIKKYAAEHDLQDFHHQLNEKSDIIQDQTYDYPASFYEPDADELAFIQFSSGSTGDPKGVMLTHHNLIHNTCAIGNALAVHSRDSFLSWMPLTHDMGLIACHLVPFITGINQNLMPTELFIRRPILWMKKAHEHKASILSSPNFGYNYFLKFLKNEPDWDLSHIKVIANGAEPILPELCDEFLKRCAAFNLKRSAILNVYGLAEASVGAAFSKLGKEFVPVYLHRDHLNLGERAVNVSKEDQNCASFVEVGRPIDYCQLRISDEANERVEDGIIGHIQIKGDNVTQGYYNNPESTEKALTSDGWVKTGDLGFISESGNLVVTGREKDIIFVNGKNIYPHDIERVAIEMEEVDLGRVAACGVYDQKTQSGEIVLFVVYKKSPEKFAPLVKEIKKHLLKRGGWSIKDVLPIRKLPKTTSGKVKRYELARQYEAGNFSTESAAINEYLESSPETSGQTPIHEIETELLSIFSDVLNGKKVHLADSYFDMGANSLQLSQIAERIEQKFGRELAVSDLFTYPSITDLAAYLSESRAEIKQDVAAKPSHVTPKDIAIIGMSLNVPGASTKNDFWNLLEKGEHSIREYPASRLKDAADYLKSIQSEINENQFVKGGYLDEIDRFDFSFFGLAPKTAQFMDPNQRLFLQSAWHAIEDAGYAGGSMNGSRVGVYAGYSKVGYDYERLLSANYPEELHQYIVGNLPSVLASRIAYFLNLKGPAVTVDTACSSSLAAVHMACKSLISGDCEMALAGGIRTSLLPICIGLDMESSDGYTKTFSKDSDGTGTGEGAAAVLLKPLQDAVRDGDHIYGVIKGSALNQDGTTAGITAPNPAAQTEVIETAWKDAGIAPETLSFIEAHGTGTKLGDPVEFNGLCKAFEKYTAKKQFCAIGSVKSNIGHLFEAAGIVGLIKSVLMLNHKKNPPLVHFNEPNPLIHFHSSPFYVNQEAAAFPSGDEPLRGGVSSFGFSGTNAHVVLEEYISQSEYAPEDEHGPHLFVLSAHTEKSLYELAQQYRQYVSDDSQASLKSICYTASTGRAHLDHGIAMIVSGKQELSDKLTRLIQGDRNLPGVYIGYKNMKEMLPAHKEELNKQAAALIKQRLRTQDERITWLHRAAELFVQGAVIDWRALYSGETVQKTPLPLYPFERSRCWAEADQLRLNEDEKRGEAALNINQSKSHIESFLKTVISNTSGIRAEELDLNAHFIGLGMDSIMLSQVKKAIADEFGADIPMDRFFDTMNNLQSVIDYLAETVPTSFASAPPQENVPAQEMQVISEAQSESDRREGHQEHMLEKIIASQNQLIQDTLQAQLNSFNLLRNSGHHSDEKEYAKAQERSIPSVQQGPPAVTAEKKAAQEAKPYVPFQPQNLHEQGHYTARQKQYLEDFIKKYADKTKGSKQYTDNTRFAHANNRNLSSFRSYWKEIVYPIIAERSDGSKMWDIDGNEYIDVTMGFGVNLFGHHPSFITQVIDDSARSSLPPLGPMSDVAGEVADRIRTCTGVERVAFYNSGTEAVMVALRLARAATGRKKVVAFSGSYHGTFDGVLGVAGTKGGAASANPLAPGILQSFMDDLIILHYNNPDSLDVIRSLGDELAAVLVEPVQSRRPDLQPRAFLKELRAITQQSGTALIMDEIITGFRIGLGGAQEWFGIQADLVTYGKIIGGGQPLGVVAGKAEFMNAIDGGTWQYGDDSYPQDEAKRTFVAGTFNTHPLTMRMSLAVLRHLQTEGEHLYEQLNQKTAYLVDELNRCFEQAQVPIRMVRFGSLFRFVSSLDNDLFFYHLNYKGVYVWEGRNCFLSAAHTADDIEKIIQAVKDTVEDLRRGGFIPEGPDSPDGGGRKKSGTRELSPEQKQLVMASHYGNEASAALNQSIMLKVEGELQHTPLKQAVRHIVGRHEALRTVIHPDDEVQQVQERMNIEIPVIDFTVHPHEHRESEIQKWLTEDAKRPFHFHEQKPLFRIHVLTSAHNEHLIVLTFHHIIADGWSIAVFVQELESNYAAIVQGKPISPKEADVSFRQYLDWQQAQIDSGHYEEGVRYWRRHFSEPIQQPILPSTGSVRYPNGYEGDRCTVRLGRPLSEALRSLSIQMKNSVFVTMLGAFHLFLHRLTKQSGLVIGIPAAGQSHIKQHDLIGNCVNMIPVKNTSTSESTLTGYLGSMKESVNLAMRHQAVPMTLVARELPHDQVPDMRIIFNLDRPFRKLHFGKAEAEPVAYPVKCTLYDLFLNITDAHQEYVLDFDFNTNVISPEIMKKWGAGFTNLLQKMVEGDSIPLDAMMMFSDEEQHDLQKLYAEHQKRVSSIGSNTANFTEAYEAPINETERQLARIWEELFGLERVGRSDRFLALGGNSLQATLMLSKIQKTFHQKVSIGQFFNHQTVKELAHFIQNETKVVHLPMKAAEKKAYYPTSPAQQRVYFLHQLEPDQLAQNMFGQISITGKYDEQALISSLQQVMQRHEAFRTYFDIIDGDIVQKLENEVDFNVHVRTMSRDEFDAYSDRFVKPFRLDQAPLVRAELIKIENEQAELLIDMHHIISDGYSVNILTNELLALYHQKPLPDIEFEYKDFAEWQNQRLNEDAMKRQETYWLEQFQDEIPILDLPTDGSKAAERSSEGQRVTCSLQPDVIRSLQDLAQKAETTLYTVLLAAYNVLLHKYTGQEDIVVGTPASGRNHPDIEKIIGIFIQTIGIRTKPHANRTFTDYLEEVKRQTLDAFENQDYPFDRLVEKLNVQRETTGKSLFNTMFVFQNIEFHEIRHNECTFKVKERNPGVSLYDLMLTIEDAGQQIEMHFDYKPGRFTKDTIEQITRHYTGILNSLVEQPEMTLSSVPMLSETERHQLLTECNGTKTPYPHNETVTRWFEMQAEQSPDHAAVIFGNERYTYRQLNERANRLARTLRTKGVQADQFVAIISPHRIELIVGILAVLKSGGAYVPIDPEYPEDRIQYMLRDSRAEVVLTQRSLLDQLPYDGDVVLLDEENSYHEDHSNLESDSDAHDLAYMIYTSGSTGNPKGVLIEHQGLADYIWWAKEVYVRGEKTNFPLYSSISFDLTVTSIFTPLVTGNTIIVFDGEDKSAVLSEIMRDSRIDMIKLTPAHLHVIKEMNIGGGTAIRKMIVGGENLSTRLAKSVSEQFKGRLDIFNEYGPTEAVVGCMIYHFDAERDKREFVPIGTPAANTDIYVADASRNLVPIGVIGEIYISGPGVARGYWNRPDLTAEKFVENPYVPGAKMYKSGDLAKRLKDGNLVYIGRVDEQVKIRGYRIELGEIEAAMHNAEAVQKAAVTVKEEEDGLKQLCAYYVSDKPIAAAQLREQLSSELPDYMVPSYFVQLEHMPLTSNGKINRKALPAPEASLQQTAEYVPPGNETESKLTDLWKEVLGISHAGIKHNFFDLGGNSIRAAALAARIHKELDVNLSLKDIFKFPTIEQLADKALHMDKNRYVPIPAAKEMPYYPVSSAQRRMYLLSHTEGGELTYNMTGAMNVEGTIDPERLNAAFRKLIARHEALRTSFDLYEGEPAQRIHQNVDFTIERIQASEEEAEDRVLDFIKAFDLAKPPLMRAGLIEIEPARHVLVVDMHHIISDGVSVNILMKDLSRIYEGNEPDPLSIQYKDFAVWQQSDIQKRNIKSQEAYWLDQFHSDIPVLDMPADYERPAIRDYEGESFEFLIPEHLKQRLSQMEEDTGATLYMILLAAYTILLSRYSGQEDIIVGTPSAGRTHLDVEPVVGMFVNTLVIRNHPAGRKTFDAYLNEVKENMLNAYKNQDYPLEELIQHLHLPKDSSRNPLFDTMFVLQNLDHAELTFDSLQLKPYSFHHPVAKFDLTLSIQADQDNYHGLFEYSKKLFKKSRIEVLSNDYLHILSAILEQPSILIEHIGLSGSNEEEENALDSIQLNF

SEQ ID NO: 11 - protein, 10 aa, Bacillus subtilis
  MISC_FEATURE (2)..(2): Xaa is Orn
  MISC_FEATURE (4)..(4): Xaa is aThr
EXYXEAPQYI

SEQ ID NO: 12 - protein, 10 aa, Bacillus subtilis
  MISC_FEATURE (2)..(2): Xaa is Orn
  MISC_FEATURE (4)..(4): Xaa is aThr
EXYXEVPQYI

SEQ ID NO: 13 - protein, 10 aa, Bacillus subtilis
  MISC_FEATURE (2)..(2): Xaa is Orn
  MISC_FEATURE (4)..(4): Xaa is aThr
EXYXEAPQYI

SEQ ID NO: 14 - protein, 10 aa, Bacillus subtilis
  MISC_FEATURE (2)..(2): Xaa is Orn
  MISC_FEATURE (4)..(4): Xaa is aThr
EXYXEVPQYI

SEQ ID NO: 15 - protein, 7 aa, Bacillus subtilis
ELLVDLL

SEQ ID NO: 16 - protein, 7 aa, Bacillus subtilis
  MISC_FEATURE (1)..(1): Xaa is Gln or Glu
  MISC_FEATURE (2)..(2): Xaa is Leu or Ile
  MISC_FEATURE (4)..(4): Xaa is Val or Ile
  MISC_FEATURE (7)..(7): Xaa is Val or Ile
XXLXDLX

SEQ ID NO: 17 - protein, 7 aa, Bacillus subtilis
  MISC_FEATURE (7)..(7): Xaa is Val or Ile
ELLLDLX

SEQ ID NO: 18 - protein, 7 aa, Bacillus subtilis
  MISC_FEATURE (2)..(2): Xaa is Val, Leu or Ile
  MISC_FEATURE (4)..(4): Xaa is Ala, Val, Leu or Ile
  MISC_FEATURE (7)..(7): Xaa is Val, Leu or Ile
EXLXDLX

SEQ ID NO: 19 - protein, 7 aa, Bacillus subtilis
NYNQPNS

SEQ ID NO: 20 - protein, 7 aa, Bacillus subtilis
NYNQPNS

SEQ ID NO: 21 - protein, 7 aa, Bacillus subtilis
DYNQPNS

SEQ ID NO: 22 - protein, 7 aa, Bacillus subtilis
NYNQPSN

SEQ ID NO: 23 - protein, 7 aa, Bacillus subtilis
NYNPEST

SEQ ID NO: 24 - protein, 7 aa, Bacillus subtilis
NYNQPNT

SEQ ID NO: 25 - protein, 7 aa, Bacillus subtilis
DYNSQST

SEQ ID NO: 26 - protein, 7 aa, Bacillus subtilis
NYNSEST

SEQ ID NO: 27 - protein, 8 aa, Bacillus subtilis
  MISC_FEATURE (6)..(6): Xaa is betaAA
NSESTXNY

SEQ ID NO: 28 - DNA, 1899 bp, Bacillus subtilis
atgggcacacttcaggagaaagtgaggcgttttcaaaagaaaaccattaccgagttaagagacaggcaaaatgctgatggttcatggacattttgctttgaaggaccaatcatgacaaattccttttttattttgctccttacctcactagatgaaggcgaaaatgaaaaagaactgatatcatcccttgcagccggcattcatgcaaaacagcagccagacggcacatttatcaactatcccgatgaaacgcgcggaaatctaacggctaccgtccaaggatatgtcgggatgctggcttcaggatgttttcacagaactgagccgcacatgaagaaagctgaacaatttatcatctcacatggcggtttgagacatgttcattttatgacaaaatggatgcttgccgcgaacgggctttatccttggcctgctttgtatttaccattatcactcatggcgctccccccaacattgccgattcatttctatcagttcagctcatatgcccgtattcattttgctcctatggctgtaacactcaatcagcgatttgtccttattaaccgcaatatttcatctcttcaccatctcgatccgcacatgacaaaaaatcctttcacttggcttcggtctgatgctttcgaagaaagagatctcacgtctattttgttacattggaaacgcgtttttcatgcaccatttgcttttcagcagctgggcctacagacagctaaaacgtatatgctggaccggattgaaaaagatggaacattatacagctatgcgagcgcaaccatatatatggtttacagccttctgtcacttggtgtgtcacgctattctcctattatcaggagggcgattaccggcattaaatcactggtgactaaatgcaacgggattccttatctggaaaactctacttcaactgtttgggatacagctttaataagctatgcccttcaaaaaaatggtgtgaccgaaacggatggctctgttacaaaagcagccgactttttgctagaacgccagcataccaaaatagcagattggtctgtcaaaaatccaaattcagttcctggcggctgggggttttcaaacattaatacaaataaccctgactgtgacgacactacagccgttttaaaggcgattccccgcaatcattctcctgcagcatgggagcggggggtatcttggcttttatcgatgcaaaacaatgacggcggattttctgctttcgaaaaaaatgtgaaccatccactgatccgccttctgccgcttgaatccgccgaggacgctgcagttgacccttcaaccgccgacctcaccggacgtgtactgcactttttaggcgagaaagttggcttcacagaaaaacatcaacatattcaacgcgcagtgaagtggcttttcgaacatcaggaacaaaatgggtcttggtacggcagatggggtgtttgctacatttacggcacttgggctgctcttactggtatgcatgcatgcggggttgaccgaaagcatcccggtatacaaaaggctctgcgttggctcaaatccatacaaaatgatgacggaagctggggagaatcctgcaaaagcgccgaaatcaaaacatatgtaccgcttcatagaggaaccattgtacaaacggcctgggctttagacgctttgctcacatatgaaaattccgaacatccgtctgttgtgaaaggcatgcaataccttaccgacagcagttcgcatagcgccgatagcctcgcgtatccagcagggatcggattgccgaagcaattttatattcgctatcacagttatccatatgtattctctttgctggctgtcgggaagtatttagattctattgaaaaggagacagcaaatgaaacgtga

SEQ ID NO: 29 - DNA, 1555 bp, Bacillus subtilis
ctttatcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctcctttcta